(19) United States
(12) Patent Application Publication
(10) Pub. No.: US 2025/0041415 A1
(43) Pub. Date: Feb. 6, 2025

(54) CYTOTOXIC INNATE LYMPHOID CELL AND USES THEREOF

(71) Applicant: Umoja Biopharma, Inc., Seattle, WA (US)

(72) Inventors: Samantha O'HARA, Loveland, CO (US); Andrew SCHARENBERG, Seattle, WA (US); Ryan CRISMAN, Denver, CO (US); Christopher NICOLAI, Seattle, WA (US); Teisha ROWLAND, Broomfield, CO (US); Ryan LARSON, Seattle, WA (US); Ryan KONING, Denver, CO (US); Christopher GARBE, Vail, CO (US); David VERIEDE, Broomfield, CO (US); Byoung RYU, Mercer Island, WA (US)

(73) Assignee: Umoja Biopharma, Inc., Seattle, WA (US)

(21) Appl. No.: 18/720,599

(22) PCT Filed: Dec. 16, 2022
(86) PCT No.: PCT/US2022/081886

§ 371 Date: Jun. 14, 2024

Related U.S. Application Data
(60) Provisional application No. 63/291,235, filed on Dec. 17, 2021.
(60) Provisional application No. 63/291,237, filed on Dec. 17, 2021.
(60) Provisional application No. 63/392,862, filed on Jul. 27, 2022.
(60) Provisional application No. 63/392,864, filed on Jul. 27, 2022.
(60) Provisional application No. 63/411,056, filed on Sep. 28, 2022.
(60) Provisional application No. 63/411,063, filed on Sep. 28, 2022.
(60) Provisional application No. 63/422,830, filed on Nov. 4, 2022.
(60) Provisional application No. 63/422,848, filed on Nov. 4, 2022.

(51) Int. Cl. A61K 39/00; C07K 14/725; C07K 16/44; C12N 5/0783

(52) U.S. Cl. CPC A61K 39/4631; A61K 39/4613; A61K 39/4646; C07K 14/7051; C07K 16/44; C12N 5/0646; C12N 2501/125; C12N 2501/2302; C12N 2501/2315; C12N 2501/26; C12N 2506/45

(57) ABSTRACT

Provided are compositions and methods for a cell population comprising engineered cytotoxic innate lymphoid cells engineered for controlled expansion and/or activity, the engineered cytotoxic innate lymphoid cells comprising a synthetic cytokine receptor for a non-physiological ligand. The cytokine receptor may comprise a synthetic gamma chain polypeptide as a first dimerization domain, a first transmembrane domain, and an interleukin-2 receptor subunit gamma (IL-2RG) intracellular domain and a synthetic beta chain polypeptide as a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. The non-physiological ligand activates the synthetic cytokine receptor in the cytotoxic innate lymphoid cells to induce expansion and/or activation of the engineered cytotoxic innate lymphoid cells.

### CROSS-REFERENCE TO RELATED APPLICATIONS

This application claims priority from U.S. provisional application No. 63/291,235, filed Dec. 17, 2021, U.S. provisional application No. 63/291,237, filed Dec. 17, 2021, U.S. provisional application No. 63/392,862, filed Jul. 27, 2022, U.S. provisional application No. 63/392,864, filed Jul. 27, 2022, U.S. provisional application No. 63/411,056, filed Sep. 28, 2022, U.S. provisional application No. 63/411,063, filed Sep. 28, 2022, U.S. provisional application No. 63/422,848, filed Nov. 4, 2022, U.S. provisional application No. 63/422,830, filed Nov. 4, 2022, the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 260132000140SeqList.xml created Dec. 16, 2022 which is 85,737 bytes in size. The information in the electronic format of the Sequence Listing is incorporated by reference in its entirety.

### TECHNICAL FIELD

The invention relates generally to engineered lymphocytes, such as cytotoxic innate lymphoid cells (CILs), methods for making such cells, and methods of using them in immunotherapy.

### BACKGROUND

Cytotoxic innate lymphoid cells (CILs) are a class of immune cells that may be used in immunotherapy including cancer immunotherapy. One type of CIL is a natural killer (NK) cell, a type of cell generally identified as positive for the cell surface protein CD56 (CD56+) and other markers and as having cytotoxic activity.

CIL cells (e.g., NK cells) for use in immunotherapy can be obtained from primary sources such as peripheral blood or umbilical cord blood. Artificial sources for CIL cells include pluripotent stem cells, including induced pluripotent stem cells (iPSCs), which are cells derived from somatic cells (generally fibroblasts or peripheral blood mononuclear cells [PBMCs]), and human embryonic stem cells (hESCs), either induced to become capable of unlimited proliferation and of differentiation into other cell types when subjected to appropriate differentiation conditions. From iPSCs, CIL cells may be derived by sequentially differentiating the iPSCs into hematopoietic progenitor cells (HPCs), also termed hematopoietic stem cells (HSCs); the HPCs into common lymphoid progenitor cells (CLPs); and then the CLPs into CIL cells-termed iPSC-derived cytotoxic innate lymphoid cells (iPSC-CILs). Generally, iPSC-CIL cells express CD56 and have cytotoxic activity, like NK cells; but iPSC-CIL cells may differ from NK cells phenotypically and in other respects.

Once obtained, primary CIL or iPSC-CIL cells can be expanded ex vivo before administration to patients. Methods for expanding CIL cells ex vivo include contacting the cells with the cytokines interleukin 2 (IL-2) or interleukin 15 (IL-15). Known expansion protocols generally combine IL-2 or IL-15 with exogenous soluble factors, bead-bound factors, or feeder cells.

Effective methods for CIL cell expansion for manufacturing purposes are known to require such factors or feeder cells. Such methods may further provide the CIL cells other factors to drive expansion. For example, antigen presenting cells (APCs) may be genetically engineered to create artificial APCs (aAPCs). Expansion is generally further enhanced by engineering the aAPCs to secrete interleukin 15 (IL-15) or interleukin 21 (IL-21); or to display recombinant membrane-bound IL-15 (mbIL-15) or membrane-bound IL-21 (mbIL-21). In sum, known methods for effective CIL cell expansion generally rely on diverse exogenous soluble and feeder cell-based factors.

Methods for differentiating iPSCs into CD34+ HPCs using either embryoid embodies (EBs) or culture of single-cell iPSCs on feeder cells are known. CD34+ HPCs may then be differentiated into CLPs, conventionally with a combination of Stem Cell Factor (SCF), Bone Morphogenetic Protein 4 (BMP4), and Vascular Endothelial Growth Factor (VEGF). Known methods of differentiation from CLPs to CILs generally involve contacting CLPs with SCF, interleukin 17 (IL-7), interleukin 15 (IL-15), FMS-like tyrosine kinase 3 ligand (FLT3L), and optionally interleukin (IL-3). The resulting iPSC-CIL cells may then be expanded using the methods already described.

There remains a need in the art for compositions and methods related to engineered cytotoxic innate lymphoid cells, methods for making such cells, and methods of using them in immunotherapy.

### SUMMARY OF THE INVENTION

The present disclosure is based, in part, on the surprising discovery by the present inventors of unexpected properties of lymphocytes, such as cytotoxic innate lymphoid (CIL) cells (e.g. NK cells), engineered to express a synthetic cytokine receptor. In some embodiments, lymphocytes, such as CIL cells (e.g. NK cells), engineered to express a synthetic cytokine receptor may expand in response to the receptor's cognate non-physiological ligand and have other desirable characteristics, such as functional activity equal to or greater than non-engineered lymphocytes, such as CIL cells, from other sources.

According to the methods described herein, lymphocytes, such as CIL cells, may be generated in high quantities and with desirable functional characteristics from primary cells or iPSC-derived lymphocytes, such as cytotoxic innate lymphoid (iPSC-CIL) cells. Non-limiting advantages of certain embodiments may include the ability of the engineered lymphocytes, such as CIL cells (e.g. NK cells), described herein to be expanded without the use of exogenous factors, such as without IL-2, IL-7, IL-15, and/or IL-21. Because it is well-known in the relevant art that expansion of CIL cells (e.g. NK cells) generally depends on having various exogenous stimuli for the CIL cells, the expansion of the engineered CIL cells omitting one or more of these stimuli, as described herein, is surprising and unexpected. In certain embodiments, the methods disclosed herein may further enhance expansion by providing other expansion factors to the engineered lymphocytes, such as engineered CIL cells. The engineered lymphocytes, such as engineered CIL cells described herein, and related compositions, may be used for immunotherapy with ex vivo expansion.

Further non-limiting advantages of the described herein may include increased proliferative capacity, resistance to senescence, cytotoxic activity compared to lymphocytes (e.g. CIL cells, such as NK cells) that do not express a synthetic cytokine receptor. In some cases, advantages also can include improved expression of surface markers characteristic of NK cells.

Among provided embodiments are an engineered lymphocyte or progenitor cell comprising a synthetic cytokine receptor for a non-physiological ligand, wherein the cytokine receptor comprises: (i) a synthetic gamma chain polypeptide comprising a first dimerization domain, a first transmembrane domain, and an interleukin-2 receptor subunit gamma (IL-2RG) intracellular domain, and (ii) a synthetic alpha or beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, an interleukin-21 receptor subunit beta (IL-21RB) intracellular domain or an interleukin-9 receptor subunit alpha (IL-9RA) intracellular domain. In some embodiments, engineering of the synthetic cytokine receptor in the engineered lymphocyte or progenitor cells provides for controlled differentiation and/or expansion of the engineered cells. In some embodiments, binding of the non-physiological ligand to the synthetic cytokine receptor activates the synthetic cytokine receptor in the engineered lymphocyte or progenitor cell to induce differentiation and/or expansion of the engineered lymphocyte and/or progenitor cell in a cell population.

In some embodiments, the engineered cell is an engineered progenitor cell that is a hematopoietic progenitor cells (HPCs; also termed hematopoietic stem cells (HSCs)) or a common lymphoid progenitor cells (CLPs). In some embodiments, the engineered cell is a cytotoxic innate lymphocyte (CIL), NK cell, or a T cell. In some embodiments, the T cell is a γδ T cell or a αβ T cell. In particular embodiments, the engineered cell is a CIL, such as an NK cell.

In some embodiments, provided herein is an engineered cytotoxic innate lymphoid (CIL) cell comprising a synthetic cytokine receptor for a non-physiological ligand, wherein the cytokine receptor comprises: a synthetic gamma chain polypeptide comprising a first dimerization domain, a first transmembrane domain, and an interleukin-2 receptor subunit gamma (IL-2RG) intracellular domain, and a synthetic alpha or beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, an interleukin-21 receptor subunit beta (IL-21RB) intracellular domain or an interleukin-9 receptor subunit alpha (IL-9RA) intracellular domain. In some embodiments, the synthetic cytokine receptor for a non-physiological ligand is engineered into the engineered CIL cell for controlled expansion and/or activity of the engineered CIL. In some embodiments, binding of the non-physiological ligand to the synthetic cytokine receptor activates the synthetic cytokine receptor in the engineered CIL cell to induce expansion and/or activation of the engineered CIL cell in a cell population.

Also provided herein are cell populations containing engineered lymphocytes or progenitor cells according to any of the provided embodiments. Also provided herein are cell populations containing engineered CIL cells according to any of the provided embodiments. Also provided herein are methods of genetically engineering the cells to express a synthetic cytokine receptor by introducing a nucleic acid or vector encoding the synthetic cytokine receptor into the cell. In some embodiments, the synthetic cytokine receptor is inserted into an endogenous gene in the cell. In such embodiments, the methods include contacting a population of lymphocytes or progenitor cells, such as CIL cells, with a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand, thereby inserting the nucleotide sequence into the endogenous gene. Any methods for gene editing and insertion of the synthetic cytokine gene, including any as described herein can be used. In some embodiments, the method of insertion is by homology directed repair (HDR). Also provided are cell populations produced by any of the provided methods.

Also provided are methods of expanding engineered lymphocytes, such as engineered CIL cells, by contacting any of the provided engineered cells with the non-physiological ligand of the synthetic cytokine receptor. Also provided are methods of expanding engineered lymphocytes, such as engineered CIL cells, by contacting any of the provided populations of engineered cells with the non-physiological ligand of the synthetic cytokine receptor. In some embodiments, the non-physiological ligand is rapamycin or a rapamycin analog.

Also provided herein are pharmaceutical compositions containing any of the provided engineered cells, such as engineered CILs. In some embodiments, the pharmaceutical composition contains a pharmaceutically acceptable carrier.

In some embodiments, also provided herein are methods of treatment and uses of any of the provided engineered cells or populations of engineered cells. Also provided herein are methods of treatment and uses of any of the provided pharmaceutical compositions. Provided herein are uses of any of the provided engineered cells, populations of engineered cells or pharmaceutical compositions in such methods and treatments, and in the preparation of a medicament in order to carry out such therapeutic methods. In some embodiments, the methods are carried out by administering the engineered cells or compositions comprising the same, to the subject having, having had, or suspected of having the disease or condition or disorder. In some embodiments, the methods thereby treat the disease or condition or disorder in the subject. In some embodiments, the methods can be used in the treatment of any disease or condition in which immunotherapy by adoptive cell therapy can be used to treat the disease or condition. In some embodiments, the engineered cells express a recombinant receptor, such as a chimeric antigen receptor (CAR) that is targeted to an antigen on a cell associated with a disease or condition. In some embodiments, the disease or condition is a cancer. Exemplary methods and uses are described herein.

In certain embodiments, provided herein are methods of treatment, kits, and pharmaceutical compositions for use in immunotherapy with ex vivo generated lymphocytes (e.g. CIL cells, such as NK cells). In certain embodiments, the engineered lymphocytes, such as CIL cells, described herein are generated, and optionally expanded ex vivo, then administered to a subject in need of immunotherapy.

In preferred embodiments, the synthetic cytokine receptor is a rapamycin-activated cytokine receptor (RACR). The RACR may be activated either with rapamycin or a derivative thereof (a rapalog).

The lymphocytes, such as CIL cells (e.g. NK cells) may be derived from iPSCs, common lymphoid progenitor cells (CLPs), or other stem or progenitor cells. Further provided herein are CLPs engineered to express synthetic cytokine receptors, and methods of differentiating engineered CLPs into CIL cells by contacting the CLPs with the cognate non-physiological ligand for the cytokine receptor.

The disclosure also provides other compositions and methods, including but not limited to pharmaceutical compositions, methods of treatment, kits, and the like. The foregoing advantages may be present in some but not all of the embodiments described herein, and other advantages of the compositions and methods described herein will become apparent from the detailed description that follows.

### BRIEF DESCRIPTION OF FIGURES

FIG. 1A is a diagram of an embodiment, showing expansion of an engineered cytotoxic innate lymphoid cell (e.g., natural killer (NK) cell).

FIG. 1B is a diagram of an embodiment, showing differentiation of an engineered stem or progenitor cell.

FIG. 2A is a panel of graphs depicting RACR-Expansion of CD19 CAR Blood-Derived NK (bdNK) cells. The top panel shows a timeline of the experiment. Percent CAR expression over time for each group is shown in the left panel and total CAR+ NK cells over time are shown in the right panel.

FIG. 2B is a panel of graphs depicting CAR expression analysis by flow cytometry. CAR expression is shown on the top panels for bdNK cells expanded in the presence of either IL-2 (1000 U/ml) or AP21967 (20 nM). CD56 and CD16 expression of the CAR+ cells are shown on the bottom panels.

FIG. 2C is a panel of graphs depicting RACR-expanded blood-derived NK cells' ability to recognize and target tumor cells. The top panel shows a timeline of the experiment. A flow cytometry plot depicting CAR expression is shown on the upper right panel at Day 31 after transduction. 1×10⁵ NK cells were incubated with 1×10⁵ K562 cells, Nalm6 cells, or Nalm6 CD19 knock out cells in the presence of brefeldin A, monensin, and anti-CD107 for 5 hours in 100 μl RPMI media in a 96 well plate. Shown are levels of CD107a (left panel) and IFNγ (middle panel) expression on AP21967-expanded CAR+ cells (AP-20) or untransduced controls. % Dead (right panel) was calculated by comparing the total number of viable, cell trace violet+ cells in each well to a non-effector control well. NK effector cells were added at varying Effector cell:Target cell (E:T) ratios.

FIG. 3A is a diagram of the iPSC-derived CIL cell (e.g., NK cell) generation process.

FIG. 3B is a graph depicting % CD34+ cells before and after cell selection and analysis at Day 12 of the differentiation process.

FIG. 3C is a panel of graphs depicting CD34+ selection by flow cytometry analysis at Day 12 of the differentiation process.

FIG. 3D is a graph depicting the percentage of CD45+ cells in leukocytes and the percentage of leukocytes in progenitor cells and CIL cells, (NK cells). The percentage of CD45+ cells are plotted as % Leukocytes, CD7+ cells are plotted as progenitor cells, and CD56+ cells are plotted as CIL cells.

FIG. 3E is a panel of graphs depicting flow cytometry analysis of differentiated leukocytes (CD45+), progenitors (CD45+/CD5−/CD7+) and CIL cells (CD45+/CD5−/CD7+/CD56+).

FIG. 3F is a panel of graphs depicting the % of leukocytes or CIL cells at Day 40 (left panel) and a flow cytometry analysis of differentiated cells at Day 40 (right panel).

FIG. 3G is a panel of graphs depicting the percentage of iCIL cells which were harvested and immunophenotyped by flow cytometry for detection of the markers: CD16, IL7R, KIR, NKp30, and NKp46 (left panel) and cytotoxicity as measured by % 4HLysis of K562 cells for various Effector cell:Target cell (E:T) ratios.

FIG. 4A is a diagram depicting the timeline of the experiment.

FIG. 4B is a panel of plots depicting representative flow cytometry analysis of CD56+ cells and TagCAR (anti-fluorescein isothiocyanate (FITC) chimeric antigen receptor (CAR)) enrichment over the course of the experiment. The x-axis is TagCAR detection by FITC-AF647 and the y-axis is side scatter. Cells analyzed following ‘Mock’ transduction, at Day 32, Day 35, Day 38, and Day 42 following transduction.

FIG. 4C is a panel of graphs depicting RACR-enrichment of TagCAR cells over time (left panel), total cell counts (middle panel) and percent of cells expressing CD45 and CD56 (right panel) over time for the BXS cell line.

FIG. 4D is a panel of graphs depicting RACR-enrichment of TagCAR cells over time (left panel), total cell counts (middle panel) and percent of cells expressing CD45 and CD56 (right panel) over time for the NHS cell line.

FIG. 5A is a panel of histograms depicting different activation markers of cytokine-differentiated Mock CIL cells (BXS line) and RACR-iCIL cells (BXS and NH5 lines). Cells were gated on CD45+ CD7+ CD5− and then plotted for activation markers CD56, CD16, NKp30, NKp40 and NKG2D.

FIG. 5B is a graph of a cytotoxicity assay depicting killing of MDA-mCherry tumor cell line by cytokine differentiated Mock CIL cells (BXS cell line) and RACR-iCIL cells (BXS and NH5 cell lines).

FIG. 6A is a diagram depicting the timeline of the experiment.

FIG. 6B is a graph depicting % RACR iCIL cells over time and total number of RACR iCIL cells over time for cells treated with expansion media containing IL-2, IL-15, IL-21, IL-18, IL-7 (Cytokine Mix) or cells treated with A/C Heterodimerizer AP21967 (AP 100 nM).

FIG. 6C is panel of flow cytometry plots depicting cells stained for CD56 clone HCD56 and RACR-FRB positive cells detected through mCherry.

FIG. 6D is a panel of graphs depicting % FITC-CAR iCIL cells over time (top panel) and total number of FLCAR iCIL cells over time (bottom panel) for cells transduced with viral constructs containing TagCAR-RACR-FRB (206) and FRB-RACR-TagCAR (205).

FIG. 6E is a panel of flow cytometry plots depicting cells stained for CD56 clone HCD56 and FLCAR positive cells.

FIG. 7A is a panel of flow cytometry plots depicting cells stained for CD56 and Tag-CAR.

FIG. 7B is a panel of histograms depicting different markers of activation and cytotoxicity including NKp30, NKp46, NKG2D, NKG2A, and CD57 in TagCAR+ RACR-expanded iCIL cells, Mock IL2-expanded CIL cells, and unstained cells.

FIG. 7C is a panel of histograms depicting CD107a secretion in response to antigen in Mock IL2-expanded CIL cells and TagCAR RACR-expanded iCIL cells.

FIG. 8 is a diagram of an illustrative CAR of the disclosure where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD3ζ.

FIG. 9 is a diagram depicting differentiation factors involved in a transition from iPSC to CIL cells alongside a timeline. Differentiation factors involved in each phase of differentiation are depicted in the outlined boxes; in bold are differentiation factors illustrative of embodiments of the disclosure. In some embodiments, beginning at week 4, cells are differentiated in media comprising IL7, IL15, SCF, FLT3L, and UM729, optionally with or without rapalog. Beginning week 6, these cells are expanded in media comprising IL7, IL15, SCF, FLT3L, UM729, and CD2/NKp46 beads, optionally with or without rapalog. In some embodiments, beginning at week 4, cells are differentiated in media comprising SCF, FLT3L, and UM729, optionally with or without rapalog. Beginning week 6, these cells are expanded in media comprising SCF, FLT3L, UM729, and CD2/NKp46 beads, optionally with or without rapalog. In some embodiments, beginning at week 4, cells are differentiated in media comprising IL7, IL15, and UM729, optionally with or without rapalog. Beginning week 6, these cells are expanded in media comprising IL7, IL15, UM729, and CD2/NKp46 beads, optionally with or without rapalog. In some embodiments, beginning at week 4, cells are differentiated in media comprising UM729, optionally with or without rapalog. Beginning week 6, these cells are expanded in media comprising UM729 and CD2/NKp46 beads, optionally with or without rapalog.

FIG. 10 is a graph depicting % of RACR CD19-CAR positive cells as a function of culture day. Blood-derived NK cells were transduced with CD19-CAR-RACR (IL-2RG/IL-2RB), CD19-CAR-RACR (IL-2RG/IL-7RB), or CD19-CAR-RACR (IL-2RG/IL-21RB) containing virus. Cells were then expanded in either 100 IU/mL human IL-2 or 100 nM AP219667 in complete media with membrane bound IL-21 (mbIL-21) 41BBL and K562 feeder cells added weekly. RACR (IL-2RG/IL-2RB) “RACR2” supported the highest expansion in AP219667 with both RACR (IL-2RG/IL-7RB) “RACR7” and RACR (IL-2RG/IL-21RB) “RACR21” supported RACR expansion to a lesser extent.

FIG. 11 is a panel of graphs depicting RACR-enrichment of CAR expressing cells over time (left panel), fold change in progenitor cells (0-4 weeks) and fold change in RACR-iCIL cells (4-9 weeks) (middle panel), and cytotoxicity as measured by % Killing of K562 cells by iCIL and RACR-iCIL cells for various Effector cell:Target cell (E:T) ratios (right panel). The top of the figure depicts a diagram of hematopoietic lineage differentiation and corresponding cell culture.

FIG. 12A is a graph depicting the % of cells comprising markers CD45+ CD5−, CD45+ CD5− CD7+, or CD45+ CD5− CD7+ CD56+ for cells thawed on Day 26 or cells analyzed on Day 29 post transduction.

FIG. 12B is a graph depicting the % of CAR+ cells comprising markers CD45+ CD5−, CD45+ CD5− CD7+, or CD45+ CD5− CD7+ CD56+ for cells thawed on Day 26 and mock transduced or cells analyzed on Day 29 post viral vector transduction.

FIG. 13A is a graph depicting the % of CAR+ iCIL cells following analysis of cells placed in media comprising IL7, IL15, SCF. FLT3L, and UM729 (Full Mix), IL7, IL15, and UM729 (IL7/IL15), or UM729 (None). Each media condition was treated with or without rapalog,

FIG. 13B is a graph depicting the Fold Change (FC) in % CAR+ iCIL cells following analysis of cells placed in media comprising IL7, IL15, SCF, FLT3L, and UM729 (Full Mix), IL7, IL15, and UM729 (IL7/IL15), or UM729 (None). Each media condition was treated with or without rapalog,

FIG. 13C is a graph depicting the total number of CAR+ iCIL cells following analysis of cells placed in media comprising IL7, IL15, SCF, FLT3L, and UM729 (Full Mix), IL7, IL15, and UM729 (IL7/IL15), or UM729 (None). Each media condition was treated with or without rapalog.

FIG. 13D is a graph depicting the Fold Change (FC) in total number of CAR+ iCIL cells following analysis of cells placed in media comprising IL7, IL15, SCF, FLT3L, and UM729 (Full Mix), IL7, IL15, and UM729 (IL7/IL15), or UM729 (None). Each media condition was treated with or without rapalog,

FIG. 14A is a panel of histograms depicting different markers including CD45, CD7, and CD56 in TagCAR+ RACR-iCIL cells, placed in media comprising IL7, IL15, SCF, FLT3L, and UM729 (Full Mix), SCF, FLT3L, and UM729 (SCF/FLT3L), IL7, IL15, and UM729 (IL7/IL15), UM729 (None), or unstained cells. Each media condition was treated with or without rapalog.

FIG. 14B is a histogram depicting the CD56 marker in progenitor cells on Day 26 (CD56−) and cells placed in media comprising IL7, IL15, SCF, FLT3L, and UM729 (Full Mix) analyzed at Day 40 (CD56+).

FIG. 15A shows a graph depicting tumor cell growth after incubating ovarian carcinoma cells with unstimulated NK cells, cytokine-stimulated NK cells or RACR-stimulated NK cells. Ovarian carcinoma cells that have not been incubated with NK cells are shown in the bold, black line. Tumor cells were reintroduced (i.e., tumor cell re-challenge) after 50 hours.

FIG. 15B shows a graph depicting tumor cell growth after incubating bladder carcinoma cells with unstimulated NK cells, cytokine-stimulated NK cells or RACR-stimulated NK cells. Bladder carcinoma cells that have not been incubated with NK cells are shown in the bold, black line. Tumor cells were reintroduced (i.e., tumor cell re-challenge) at 50 and 100 hours.

FIG. 15C shows a graph depicting tumor cell growth after incubating breast adenocarcinoma cells with unstimulated NK cells, cytokine-stimulated NK cells or RACR-stimulated NK cells. Breast adenocarcinoma cells that have not been incubated with NK cells are shown in the bold, black line. Tumor cells were reintroduced (a.k.a., tumor cell re-challenge) after 40 hours.

FIG. 15D shows a graph depicting RACR-NK cell growth after incubating without rapamycin or cytokines (unstimulated NK), with rapamycin (RACR-stimulated NK) or with cytokines (cytokine-stimulated).

FIG. 16A shows a graph depicting tumor cell killing in tumor cells incubated without RACR engineered iCILs (Tumor Only or T), with RACR engineered iCILs and no CAR antigen expression (R+NCA), RACR engineered iCILs and medium CAR antigen expression (R+MCA), or RACR engineered iCILs and high CAR antigen expression (R+HCA). The CAR antigen is specific to FITC conjugated to folate, wherein the folate is bound to folate receptor on the tumor cells.

FIG. 16B shows a graph depicting the function of iPSC-derived NK cells and CAR and RACR engineered iPSC-derived CILs. The CAR-RACR-iCIL cells secrete CD107a in response to an antigen recognized by the cells.

FIG. 16C shows a graph depicting RACR-CAR-NK cell growth after incubating without rapamycin or cytokines (unstimulated NK), with rapamycin (RACR-stimulated NK) or with cytokines (cytokine-stimulated).

FIG. 17A shows a timeline of an in vivo mouse model of breast cancer. Five days before the experiment began, mice were injected with MDA-231 mCherry/luciferase cells. One day before the experiment began, mice were injected with FITC-Folate subcutaneously for two weeks. On the first day of the experiment, mice were injected with RACR-TagCAR-NK-92 cells intraperitoneally and subsequently treated with rapamycin three times per week or IL2/IL15 three times per week.

FIG. 17B shows a graph depicting quantification of luminescence correlating to tumor growth in mice receiving various treatments over three weeks.

FIG. 17C shows a raw luminescence images of tumor growth in mice receiving various treatments over three weeks.

FIG. 17D depicts RACR-NK in the blood of mice in FIG. 22C.

FIG. 17E depicts RACR-NK detection in tissues of mice in FIG. 22C.

FIG. 18 depicts tumor cell killing in vitro in co-culture of RACR iCILs with MDA-MB-231 breast adenocarcinoma cells. Breast cancer cells were cultured alone and left untreated, treated with cytokines or rapalog (left legend), or co-cultured with RACR iCILs and left untreated, treated with cytokines or rapalog (right legend).

FIG. 19A depicts a timeline of an in vivo mouse model of breast cancer. Five days before the experiment began, mice were injected with MDA-231 mCherry/luciferase cells. One day before the experiment began, mice were imaged and blood was drawn once a week for the duration of the experiment. On the first day of the experiment (DO), mice were injected with RACR-iCIL cells intraperitoneally and subsequently treated with rapamycin three times per week or IL2/IL15 three times per week.

FIG. 19B depicts raw luminescence images of tumor growth in mice receiving various treatments over three weeks.

FIG. 19C depicts a timeline of an in vivo mouse model of breast cancer. Five days before the experiment began, mice were injected with MDA-231 mCherry/luciferase cells. One day before the experiment began, mice were imaged once a week for the duration of the experiment. On the first day of the experiment (DO), mice were injected with RACR-iCIL cells and subsequently treated with rapamycin three times per week or IL2/IL15 three times per week. On day 27 of the experiment, mice were injected again with RACR-iCIL cells.

FIG. 19D depicts raw luminescence images of tumor growth in mice receiving various treatments over six weeks.

FIG. 19E shows a survival plot of mice described in FIG. 19D.

FIGS. 20A-20D show a panel of graphs depicting RACR-dependent expansion of Vd2 cells. The percentage of RACR+ Vd2+ expression over time for each treatment group (e.g., IL2, RAPA, AC) is shown in FIG. 20A. The total number of RACR+Vd2+ cells over time is shown in FIG. 20B. Fold-change of FIG. 20A and FIG. 20B are shown in FIG. 20C and FIG. 20D, respectively.

### DETAILED DESCRIPTION OF THE INVENTION

Induced pluripotent stem cells (iPSCs) are a renewable, modifiable, and scalable source of material for cell therapy manufacturing. However, major challenges exist in current iPSC-based approaches to cell therapy. Specifically, current approaches for differentiating iPSCs into therapeutic immune cell types require exogenous growth factors, and in some cases, the presence of feeder cells.

iPSCs can be made by reprogramming adult cells into a cellular state akin to embryonic stem cells. iPSCs are thought to be capable of differentiating into all cell types found in the human body and possess an unlimited expansion capacity, meaning they can reproduce and proliferate indefinitely, generating a nearly endless supply of starting material. Additionally, iPSCs are amenable to precision multiplex genome editing, allowing safe introduction of multiple genetic modifications. Because of these properties, iPSCs provide a consistent starting material, originating from a single cell (clone), which enables consistent genome integrity in process intermediates and the final cell product.

Current approaches to cell therapy manufacturing include either autologous or allogeneic cellular starting material from which a disease-targeted therapeutic cell product is engineered. In some embodiments, an allogeneic or “off-the-shelf” cell therapy has the potential to transform cell therapy from personalized medicine into a routine treatment. However, current “off-the-shelf” cell therapies are struggling to show the same engraftment and persistence of cells in vivo that has been reached by approved autologous cell therapy products. In some aspects, this is due to the foreign nature of allogeneic or even engineered elements of autologous cells, which can be recognized and rejected by the host immune system. To date, cell therapies achieve cell engraftment by treating patients with highly toxic chemotherapy regimens, termed lymphodepletion (LD), given prior to administration of the cell therapy product. LD essentially removes the host immune system and provides many benefits to the cell therapy product, firstly providing free “homeostatic cytokines” for an ex vivo cell therapy product as well as reducing anti-graft responses against the foreign graft by the host immune system. However, LD is a transient solution, and the host immune system rapidly reconstitutes. Thus, to sustain allogeneic cell exposure, multiple rounds of LD and cell infusion are required. Additionally, exogenous cytokines such as IL-2 are administered, and these cytokine treatments have low exposure times with high toxicities associated with their use. Finding better ways to increase cell persistence is key to achieving durable tumor remission and has proven to be a challenge in the allogeneic cell therapy space.

Allogeneic cells can be further broken down into donor- or iPSC-derived cells. Donor-derived cells are generally sourced from the circulation or cord blood of a healthy donor and the therapeutic cell type (e.g., natural killer or NK cells) is selected, subsequently harvested, and expanded in a complex cell culture process that generally includes multiple cytokines, growth factors, gene engineering, and feeder cells to generate many doses. Alternatively, iPSC-derived cells, which also require multiple complex cell culture conditions, must have these conditions implemented in a stepwise fashion to drive cells through the necessary progenitor stages to ultimately obtain the intended final cell product (e.g., immune effector cell). For example, effective methods for CIL cell expansion for clinical-scale purposes typically involve exogenous cytokines, including IL-2, IL-15, and/or IL-7, as well as antigen molecules, co-stimulatory molecules, and/or cell adhesion molecules. CIL cells are cytotoxic lymphocytes characterized by their ability to discriminate between self and non-self by monitoring the expression of MHC class I molecules, release of cytokines, and directly kill non-self or infected target cells. It is known in the art that CIL cells do not represent a uniform population. Rather, there are many distinguishable subsets of CIL cells. In many studies, these exogenous factors are supplemented during ex vivo expansion of CIL cells and/or after infusion of the CIL cells into a subject. Effectively expanding CIL cells that rely on a large quantity of diverse exogenous factors using currently known methods often requires complex and expensive manufacturing processes. A further difficulty in the field involves modulating the activity of endogenous CIL cells in vivo, especially given that patients with cancer exhibit significantly reduced NK cell activity as compared with healthy patients.

Furthermore, the gold standard for cell therapy is autologous chimeric antigen receptor (CAR) T cell therapy. Decades of CAR T cell therapy efforts, starting with “first generation” CAR T cell therapies in the 1990s and leading to the first CAR T cell therapies receiving FDA approval in 2017, have resulted in successes in treating B cell malignancies, with long-term remission achieved in 30-40% of certain patient populations. Importantly, CAR T cell efficacy requires lymphodepleting chemotherapy to eliminate sinks for survival factors such as IL-15. While CAR T cell therapies have revolutionized the treatment of malignancies (e.g., hematologic), major limitations hinder its widespread application. The allogeneic CAR T therapy field has shown promising early clinical results; however, the durable response profile has been generally poor in comparison to autologous CAR T cell therapies, despite the use of ever-increasing intensity LD regimens. This is likely due to limitations of the drug product cell type, manufacturing processes, as well as anti-allograft responses against the therapeutic cells. Thus, despite the promising clinical efficacy of CAR T cells in hematologic malignancies, significant challenges remain, including patient access, complex manufacturing, and high cost. The provided engineered CIL cells and methods related to the same provide for “off-the-shelf” cancer therapies to overcome these challenges.

iPSCs can also be modified via CRISPR to express a CAR to overcome challenges associated with targeting, for example, the heterogeneous solid tumor microenvironment.

Overall, iPSC-based cell therapy is generally inefficient in generating the necessary intermediate progenitor cells, resulting in a low initial yield of the therapeutic cell type (e.g., cytotoxic innate lymphocyte (CIL) cells), which then requires feeder cell-driven expansion. This feeder cell-driven expansion can dramatically reduce the proliferative capacity of the final cell therapy product. Thus, in order to achieve the necessary engraftment to have any therapeutic effect, high cell numbers (˜1 billion cells) and repeat dosing are required in addition to repeated cycles of lymphodepleting chemotherapy.

Provided herein is a platform for expanding immune effector cells in the absence of exogenous cytokines and feeder cells by genetically modifying lymphocytes, such as CILs (e.g. NK cells), to express a synthetic cytokine receptor. In some aspects, the lymphocytes can be primary cells or can be a lymphoid progenitor cell or a cell that is derived or differentiated from a lymphoid progenitor cell. In some embodiments, provided herein is a platform for expanding immune effector cells in the absence of exogenous cytokines and feeder cells by genetically modifying iPSC-derived progenitor cells (e.g. common lymphoid cells) to express a synthetic cytokine receptor for expansion of differentiation into lymphocytes, such as CILs. A synthetic small molecule ligand (e.g., rapamycin) activates the receptor to drive the expansion of immune effector cells. In some embodiments, the synthetic cytokine receptor is rapamycin activated cytokine receptor (RACR) that can be engaged by rapamycin or an analog, e.g., rapalog. In some embodiments, a RACR-induced lymphocyte is a lymphocyte type driven by RACR, either during differentiation and/or expansion. Examples of such “RACR-induced” cells include cytotoxic innate lymphocyte (RACR-iCIL), NK (RACR-iNK), or T cells (RACR-iT), including γδ T cell, or αβ T cell. In particular embodiments, the RACR-induced cell, such as RACR-iCIL, is a resultant cell that has used RACR for differentiation or expansion in place of normal cytokine pathways, such as in place of normal cytokine pathways from a progenitor cell (e.g. common lymphoid progenitor cell or a pre-NK cell).

The compositions and methods provided herein comprise lymphocytes, such as CIL cells (e.g. NK cells) engineered to express a synthetic cytokine receptor. Non-limiting advantages of the engineered lymphocytes, such as CIL cells (e.g. NK cells) include superior and controllable expansion when administered to a subject, similar cytotoxic activity as compared to native lymphocytes (e.g. CIL cells), improved iPSC-derived cell manufacturing and enhanced anti-tumor activity.

Improved Cell manufacturing. In provided aspects, the RACR engineering platform provided herein improves cell therapy manufacturing by controlling cell production. In some aspects, the RACR engineering platform provided herein improves iPSC-derived cell manufacturing by controlling cell production. The RACR engineering platform also reduces manufacturing costs as RACR activation eliminates the need to add expensive growth factors, cytokines and other raw materials. In certain embodiments, the methods disclosed herein may further enhance expansion through the ability of the engineered lymphocyts, such as CIL cells, described herein to be expanded without or with fewer exogenous factors, such as without IL-2, IL-15, and/or IL-7. The RACR engineering platform increases yields of highly pure intermediate and final cell products. The RACR engineering platform increases patient-compatibility of the cells as the manufacturing process is completely feeder cell and xenogeneic cell free. The RACR engineering platform is also compatible with cells in suspension, promoting scalability of cell production.

Enhanced Anti-Tumor Activity. In provided aspects, the RACR engineering platform provided herein improves anti-tumor activity of the engineered lymphocytes, such as CILs, including iPSC-derived cells, by increasing cell engraftment, persistence and effector function. The RACR engineering platform provided herein also improves anti-tumor activity of the engineered lymphocytes, such as CILs, including iPSC-derived cells, by inhibiting host immune response via rapamycin dosing, which further enables engraftment of the cells (e.g., CILs). The RACR engineering platform provided herein also improves anti-tumor activity of the engineered lymphocytes, such as CILs, including iPSC-derived cells by removing the need for toxic lymphodepletion (LD) due by activating the RACR system to selectively support RACR cell expansion and survival.

In some aspects, among advantages of the RACR system on lymphocytes, such as CILs (e.g. NK cells), including the engineered synthetic cytokine receptor and activation thereof with rapamycin or rapalog, includes: the ability to engineer unlimited starting material that is efficient at generating immediate progenitors and that is characterized by minimized expansion requirements on the final cell type; the ability to efficiently edit cells; no requirement for feeder cells, thereby minimizing complex, raw materials; no requirement for lymphodepletion in subjects receiving RACR-engineered cells; low to no cytokine release syndrome (CRS) or Immune effector cell-associated neurotoxicity syndrome (ICAN); and promotion of engraftment, expansion, and persistence with administration of rapamycin or rapalogs. In some embodiments, the engineered lymphocytes, such as RACR-iCILs, provide a source of cells for allogenic cell therapy, which, in some aspects, can be achieved while minimizing or eliminating hypoimmune engineering requirements.

Provided here are synthetic cytokine receptors, e.g. RACR, that can be applied to support the derivation of cytotoxic innate lymphoid (CIL) cells, e.g., NK cells. CIL cells may be derived from stem or progenitor cells, and such cells are termed herein “induced cytotoxic innate lymphoid” (iCIL) cells. iCIL cells share distinguishing cell surface markers and functional attributes as described herein. As used herein, the terms “induced cytotoxic innate lymphoid cell” or “iCIL” refers to a CIL made by inducing differentiation of progenitor cells. As disclosed herein, iCIL may be made and/or expanded by expressing a synthetic cytokine receptor in a stem or progenitor cell and acting the synthetic cytokine receptor by the non-physiological ligand. Such a process may involve differentiation of a progenitor cell (e.g. lymphoid progenitor cell) that is engineered to express a synthetic cytokine receptor by activation of the synthetic cytokine receptor. The process may also or alternatively involve expansion of the progenitor cell or the CIL by activation of the synthetic cytokine receptor.

In some embodiments, provided herein are synthetic cytokine receptors, e.g. RACR, that can be derived to support derivation or expansion of NK cells. NK cells derived from endogenous NK cells are termed herein primary NK cells. NK cells may also be derived from stem or progenitor cells, and such cells are termed herein iPSC-derived Natural Killer (INK) cells. Primary NK cells and iNK cells share distinguishing cell surface markers and functional attributes but are not necessarily identical.

In preferred embodiments, the disclosure provides CIL cells, such as NK cells, that are engineered to express a rapamycin activated cytokine receptor (RACR), a synthetic cytokine receptor activated by the small molecule rapamycin or rapalogs. CIL cells comprising a RACR and activated with rapamycin or a rapalog are termed herein “RACR-iCIL” cells, e.g., RACR-iNK cells. RACR is demonstrated to support differentiation and/or expansion of RACR-iCIL cells in a feeder-free manufacturing process. RACR-iCIL cells express multiple innate tumor targeting receptors and when engineered to express a chimeric antigen receptor (CAR), are able to exert CAR-directed cytolytic activity. Accordingly, RACR-iCIL cells provide an “off-the-shelf” allogeneic cell therapy.

The disclosure relates, in part, to the surprising discovery by the present inventors that lymphocytes, such as cytotoxic innate lymphoid (CIL) cells (e.g. NK cells) engineered to express a synthetic cytokine receptor may expand in response to the receptor's cognate non-physiological ligand. The engineered CIL cells may be generated in high quantities and with functional activity equal to or greater than CIL cells from other sources.

The disclosure relates, in part, to the surprising discovery by the present inventors that common lymphoid progenitor (CLP) cells engineered to express a synthetic cytokine receptor may be differentiated into CIL cells in response to the receptor's cognate non-physiological ligand without exogenous factors and the resulting CIL cells display a cytotoxic phenotype. It is well-known in the relevant art that expansion of CIL cells generally depends on having various exogenous stimuli. The engineered CIL cells described herein have the ability to be expanded without or with fewer exogenous factors, such as without IL-2, IL-15, and/or IL-7.

The engineered CIL cells described herein, and related compositions, may be used for immunotherapy with ex vivo expansion.

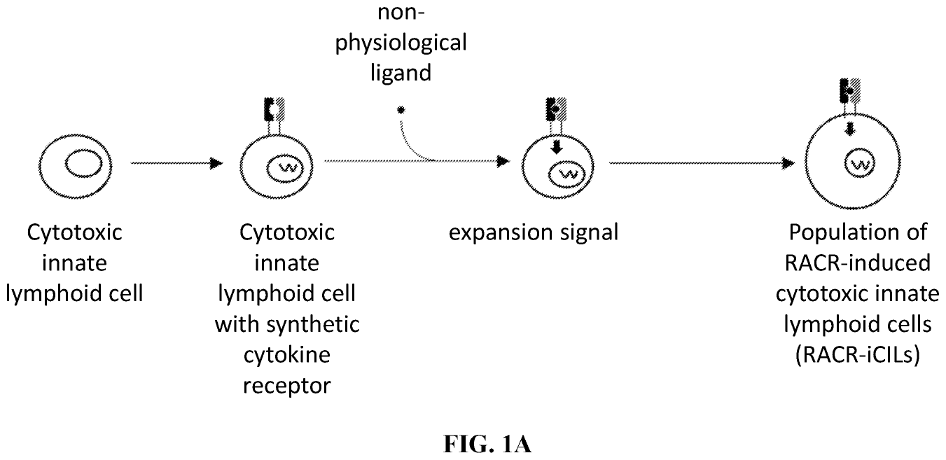

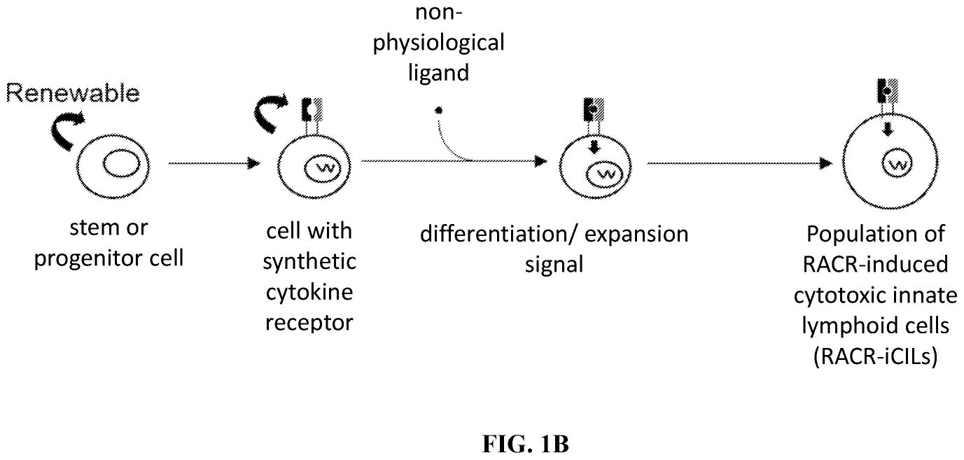

As shown in the diagram of FIG. 1A, an isolated lymphocyte, such as CIL cells (e.g., NK cells), including such primary cells isolated from peripheral blood, may be transduced with a vector comprising at least one polynucleotide encoding a synthetic cytokine receptor, such as RACR. Upon contacting the transduced lymphocyte cell, e.g. CIL cell, with a non-physiological ligand, the extracellular domains of the cytokine receptor dimerize through mutual binding of the non-physiological ligand. This dimerization generates an expansion signal within the cell, e.g. CIL cell, which produces a population of phenotypically enriched and functionally active, engineered cells, such as engineered CIL cells.

As shown in the diagram of FIG. 1B, a stem or progenitor cell may be transduced with a vector comprising at least one polynucleotide encoding a synthetic cytokine receptor, such as RACR. Upon contacting the transduced cell with a non-physiological ligand, the extracellular domains of the cytokine receptor dimerize. The dimerization generates a differentiation signal within the stem or progenitor cell which induces sequential differentiation to becoming CIL cells, such as NK cells.

The CIL cells may be derived from iPSCs, common lymphoid progenitor cells (CLPs), or other stem or progenitor cells. Further provided herein are stem or progenitor cells engineered to express synthetic cytokine receptors, and methods of differentiating engineered stem or progenitor cells into CIL cells by contacting the stem or progenitor cells with the cognate non-physiological ligand for the cytokine receptor.

In certain embodiments, provided herein are ex vivo generated lymphocytes. In some embodiments, the lymphocytes or a progenitor cell (common lymphoid progenitor or a pre-NK cell) are engineered with a provided synthetic cytokine receptor (e.g. RACR). In such aspects, a non-physiologic ligand of the synthetic cytokine receptor can be used for expansion and/or differentiation of the lymphocytes or progenitor cells ex vivo to produce a population of the ex vivo generated lymphocytes. The lymphocytes include T cells, innate lymphoid cells (ILCs), including cytotoxic innate lymphoid cells (CILs) such as natural killer (NK) cells. In some embodiments, the T cells are αβ T cells. In some embodiments, the T cells are γδ T cells.

In certain embodiments, provided herein are ex vivo generated CIL cells. In some embodiments, the CIL cells or a common lymphoid progenitor cell are engineered with a provided synthetic cytokine receptor (e.g. RACR). In such aspects, a non-physiologic ligand of the synthetic cytokine receptor can be used to expand or differentiate the cells to produce ex vivo generated CIL cells.

The engineered cells described herein, and related compositions, may be used for immunotherapy with ligand-controlled ex vivo expansion. Further provided herein are methods of expanding lymphocytes, e.g. CIL cells, that have been engineered with the synthetic cytokine receptor (e.g. RACR) by contacting the cells with the cognate non-physiological ligand for the synthetic cytokine receptor. Moreover, the engineered lymphocytes, such as engineered CIL cells, disclosed herein may be further engineered to express a chimeric antigen receptor (CAR), enabling targeting of the engineered lymphocyte, such as engineered CIL cells, to cells expressing or labelled with the antigen recognized by the CAR.

In some embodiments, the provided engineered lymphocytes, such as engineered CIL cells, and methods provided for an improved immunotherapy compared to existing strategies. While chimeric antigen receptor (CAR) T cell therapies have revolutionized the treatment of hematologic malignancies, major limitations hinder their widespread application.

In some embodiments, engagement of the synthetic cytokine receptor (e.g. RACR) is able to increase cell growth and promote expansion through engagement of the synthetic cytokine receptor on provided engineered lymphocytes, such as CIL cells. In some aspects, the provided engineered cells, including iCIL cells, and related methods can be used to increase cell growth and expansion in vivo of the engineered cell therapy through dosing of patients with the non-physiological ligand of the synthetic cytokine receptor (e.g. rapamycin dosing of patients) after the cell therapy product. In some embodiments, the non-physiological ligand (e.g. rapamycin) simultaneously expands and protects the cells. In some embodiments, expansion is achieved through the JAK/STAT signal activation and protection is achieved through rapamycin suppression of host anti-graft responses. In some embodiments, the need for lymphodepletion as well as exogenous cytokine dosing is not necessary. In some embodiments, provided methods of administration and treatment with the engineered lymphocytes, such as engineered iCIL cells, can be carried out without lymphodepletion (e.g., without the need to administer a lymphodepleting therapy such as cyclophosphamide and/or fludarabine). In some embodiments, provided methods of administration and treatment with the engineered lymphocytes, such as engineered iCIL cells, can be carried out without exogenous cytokine administration (e.g., without the need to administer IL-2 and/or IL-15).

Accordingly, provided embodiments employing a synthetic cytokine receptor system, such as a rapamycin activated cytokine receptor (RACR) that can be engaged by rapamycin or an analog, e.g., rapalog, both protects and expands cells in a single technology. In addition, the additional inclusion of genetic disruption, such as knockout of certain immune genes such as beta-2-microgloublin (B2M), also can produce “stealth” cells that have additional advantages for allogeneic cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. Definitions

All publications, patent applications, patents and other references mentioned herein are incorporated by reference in their entirety.

Unless the context indicates otherwise, the various features described herein can be used in any combination with any feature or combination of features set forth herein, and each feature can be excluded or omitted from the combination.

As used herein, the singular forms “a”, “an”, and “the” are include the plural forms as well, unless the context indicates otherwise. The conjugation “and/or” denotes all possible combinations of one or more of listed items.

“Subject” as used herein refers to the recipient of an engineered CIL cell or other agent. The term includes mammal, such as primate, mouse, rat, dog, cat, cow, horse, goat, camel, sheep or a pig, preferably a human.

“Treat,” “treating” or “treatment” as used herein refers to any type of action or administration that imparts a benefit to a subject that has a disease or disorder, including improvement in the condition of the patient (i.e., improvement, reduction, or amelioration of one or more symptoms, and partial or complete response to treatment).

The term “effective amount” refers to an amount effective to generate a desired biochemical, cellular, or physiological response. The term “therapeutically effective amount” refers to the amount, dosage, or dosage regime of a therapy effective to cause a desire treatment effect.

“Polynucleotide” as used herein refers to a biopolymer composed of two or more nucleotide monomers covalently bonded through ester linkages between the phosphoryl group of one nucleotide and the hydroxyl group of the sugar component of the next nucleotide in a chain. DNA and RNA are non-limiting examples of polynucleotides.

“Polypeptide” as used herein refers to a polymer consisting of amino acid residues chained together by peptide bonds, forming part of (or the whole of) a protein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The term “variant” means a polynucleotide or polypeptide having at least one substitution, insertion, or deletion in its sequence compared to a reference polynucleotide or polypeptide. A “functional variant” is a variant that retains one or functions of the reference polynucleotide or polypeptide.

As used herein the term “sequence identity”, or “identity” in relation to polynucleotides or polypeptide sequences, refers to the extent to which two optimally aligned polynucleotides or polypeptide sequences match at each position in the alignment across the full length of the reference sequence. The “percent identity” is the number of matched positions in the optimal alignment, divided by length of the reference sequence plus the sum of the lengths of any gaps in the reference sequence in the alignment. The optimal alignment is the alignment that results in the maximum percent identity. Alignment of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.12.0 using default parameters. And, unless noted otherwise, the alignment is an alignment of all or a portion of the polynucleotide or polypeptide sequences of interest across the full length of the reference sequence.

As used herein, “small molecule” refers to a low molecular weight (<1000 Daltons), organic compound. Small molecules may bind specific biological macromolecules and can have a variety of biological functions or applications including, but not limited to, serving as cell signaling molecules, drugs, secondary metabolites, or various other modes of action.

The term “analog” in relation to a small molecule refers to a compound having a structure and/or function similar to that of another compound but differing from it in respect to a certain component. The analog may differ in one or more atoms, functional groups, or substructures, which are replaced with other atoms, groups, or substructures. Despite a high structural and/or functional similarity, analogs can have different physical, chemical, physiochemical, biochemical, or pharmacological properties.

The term “rapalog” is an art-recognized group of analogs of rapamycin analog that share structural and functional similarity to rapamycin. Certain rapalogs are known to share some but not all functional attributes of rapamycin. For example, some rapalogs are suitable for uses as a non-physiological ligand because they promote dimerization but have substantially no immunosuppressive activity (e.g., AP21967, AP23102, or iRAP).

An illustrative rapalog of the disclosure is AP21967

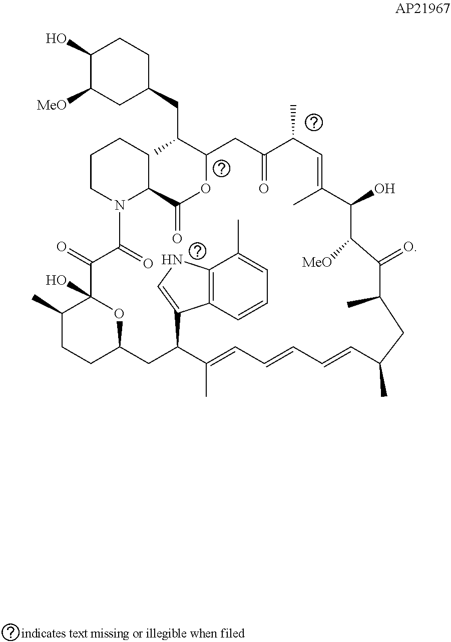 [SMILES: CO[C@@H]1C[C@H](C[C@@H](C)C2CC(=O)[C@H](C)/C=C(\C)[C@@H](O)[C@@H](OC)C(=O)[C@H](C)C[C@@H](C)/C=C/C=C/C=C(\C)[C@H](c3c[nH]c4c(C)cccc34)C[C@H]3CC[C@@H](C)[C@@](O)(O3)C(=O)C(=O)N3CCCC[C@H]3C(=O)O2)CC[C@@H]1O]

An illustrative rapalog of the disclosure is AP23102

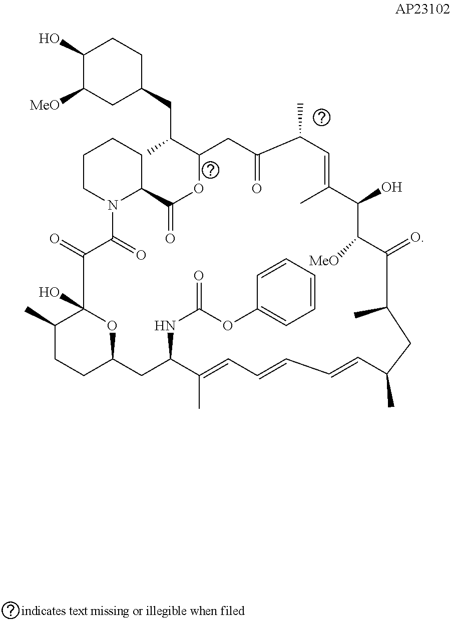 [SMILES: CO[C@@H]1C[C@H](C[C@@H](C)C2CC(=O)[C@H](C)/C=C(\C)[C@@H](O)[C@@H](OC)C(=O)[C@H](C)C[C@@H](C)/C=C/C=C/C=C(\C)[C@H](NC(=O)Oc3ccccc3)C[C@H]3CC[C@@H](C)[C@@](O)(O3)C(=O)C(=O)N3CCCC[C@H]3C(=O)O2)CC[C@@H]1O]

An illustrative rapalog of the disclosure is iRAP

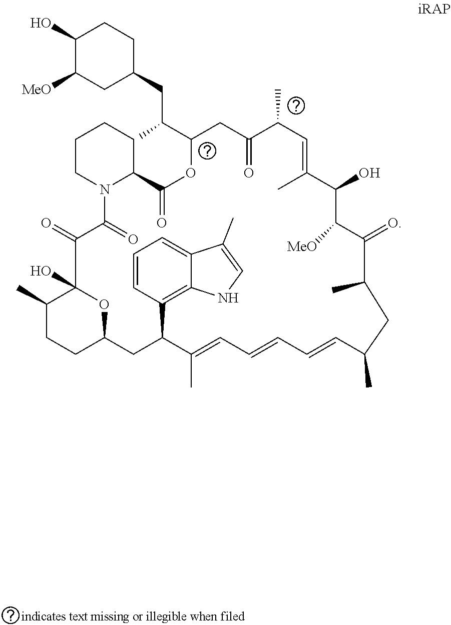 [SMILES: CO[C@@H]1C[C@H](C[C@@H](C)C2CC(=O)[C@H](C)/C=C(\C)[C@@H](O)[C@@H](OC)C(=O)[C@H](C)C[C@@H](C)/C=C/C=C/C=C(\C)[C@H](c3cccc4c(C)c[nH]c34)C[C@H]3CC[C@@H](C)[C@@](O)(O3)C(=O)C(=O)N3CCCC[C@H]3C(=O)O2)CC[C@@H]1O]

The term “cell population” refers to mixture of cells suspended in solution, attached to a substrate, or stored in a container. The characteristics of a cell population as a whole can be studied with bulk measurements of sample volumes having a plurality of cells. Flow cytometry methods may be employed to reduce problems with background fluorescence which are encountered in bulk cell population measurements.

As used herein, the term “Cytotoxic Innate Lymphoid cell” or “CIL cell” is used to refer to a class of cytotoxic lymphocytes that constitute a major component of the innate immune system. In humans, cytotoxic innate lymphoid cells usually express the surface markers CD16 (FCyRIII), CD56, and may express CD127. They may express one or more of CD45, CD94, CD122, KIR, NKG2A, NKG2D, NKp30, NKp44, NKP46, NKp80. Cytotoxic innate lymphoid cells generally do not express CD3, or express lower levels of CD3 than CD3+ T cells. CIL cells are cytotoxic and comprise small granules in their cytoplasm that contain special proteins such as perforin and proteases known as granzymes. CIL cells provide rapid responses to virally infected cells and respond to transformed cells. Upon release in close proximity to a cell slated for killing, perforin forms pores in the cell membrane of the target cell through which the granzymes and associated molecules can enter, inducing apoptosis. CIL cells may act as effectors of lymphocyte cell populations in anti-tumor and anti-infection immunity. In some embodiments, the CIL cell is a NK cell. In some embodiments, the CIL cell is a blood derived NK cell (bdNK), iPSC derived NK cell (iPSC-NK), or other cytotoxic innate lymphoid cell. For clarity, the term “CIL cells” excludes adaptive immune cells and their progenitors and excludes common lymphoid progenitor cells (CLPs). However, CILs may be derived from CLPs.

As used herein, the term “engineered” refers to a cell that has been stably transduced with a heterologous polynucleotide or subjected to gene editing to introduce, delete, or modify polynucleotides in the cell, or cells transiently transduced with a polynucleotide in a manner that causes a stable phenotypic change in the cell.

As used herein, the term “stem cell” is used to describe a cell with an undifferentiated phenotype, capable, for example, of differentiating into common lymphoid progenitors, cytotoxic innate lymphoid cells, and/or NK cells.

As used herein, the term “pluripotent” means the stem cell is capable of forming substantially all of the differentiated cell types of an organism, at least in culture. For example, embryonic stem cells are a type of pluripotent stem cells that are able to form cells from each of the three germs layers, the ectoderm, the mesoderm, and the endoderm.

As used herein, the terms “induced pluripotent stem cell” and “iPSC” are used to refer to cells, derived from somatic cells, that have been reprogrammed back to a pluripotent state and are capable of proliferation, selectable differentiation, and maturation. iPSCs are stem cells produced from differentiated adult, neonatal, or fetal cells that have been induced or changed, i.e., reprogrammed, into cells capable of differentiating into tissues of all three germ or dermal layers: mesoderm, endoderm, and ectoderm. The iPSCs produced do not refer to cells as they are found in nature.

As used herein, the term “hematopoietic stem cell” refers to stem cells capable of giving rise to both mature myeloid and lymphoid cell types including natural killer cells, T cells, and B cells. Hematopoietic stem cells are typically characterized as CD34+.

The term “progenitor” refers to a cell partially differentiated into a desired cell type. Progenitor cells retain a degree of pluripotency and may differentiate to multiple cell types.

As used herein, the term “hematopoietic progenitor cell” refers to cells of an intermediate cell type capable of differentiating down blood cell lineages, wherein the hematopoietic progenitor cell may differentiate into either common myeloid progenitor cells or common lymphoid progenitor cells. Hematopoietic progenitor cells are typically characterized as CD34+ and CD45+. CD38 is also considered a marker for hematopoietic progenitor cells. CD45 is considered a hematopoietic lineage marker.

As used herein, the terms “lymphoid progenitor cell” or “lymphoblast” or “common lymphoid progenitor” refer to cells that are precursors to lymphoid cells, e.g., CIL and NK cells. Lymphoid progenitor cells are the first stage of differentiation of hematopoietic stem cells that follow the lymphoid lineage of differentiation. As used herein, the term “lymphoid progenitor” refers to cells capable of hematopoietic transition to hematopoietic cell-types. Lymphoid progenitor cells may be characterized by being CD45+ CD7+ CD5+/lo CD3− CD56−. Lymphoid progenitor cells may be characterized by being CD45+ CD5+/lo CD7+.

As used herein, “differentiate” or “differentiated” are used to refer to the process and conditions by which undifferentiated, or immature (e.g., unspecialized), cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells.

As used herein, “expand” or “expansion” refer to an increase in the number and/or purity of a cell type within a cell population through mitotic division of cells having limited proliferative capacity, e.g., CIL cells.

As used herein, “activity”, “activate”, or “activation” refer to stimulation of activating receptors on a cytotoxic innate lymphoid cell leading to cell division, cytokine secretion (e.g., IFNγ and/or TNFα), and/or release of cytolytic granules to regulate or assist in an immune response.

### II. Source Cells and Synthetic Cytokine Receptor (e.g., RACR) Engineered Cells

In some aspects, provided herein are engineered cells containing a synthetic cytokine receptor for a non-physiological ligand, such as a synthetic small molecule ligand (e.g., rapamycin), in which the ligand is able to activate the synthetic cytokine receptor to drive the differentiation and/or expansion of the engineered cells. In some embodiments, the engineered cells are immune effector cells. In some embodiments, the engineered cells are lymphocytes. In some embodiments, lymphocytes include T cells, innate lymphoid cells (ILCs), including cytotoxic innate lymphoid cells (CILs) such as natural killer (NK) cells. In some embodiments, the T cells are αβ T cells. In some embodiments, the T cells are γδ T cells. In some embodiments, the engineered cells are progenitor cells, such as a common lymphoid progenitor cell, that are able to be differentiated into lymphocytes, such as CILs or NK cells. Also provided are resulting populations of cells induced to differentiate or expand from such engineered lymphocytes or progenitor cells. In some embodiments, the population of cells are iCILs.

In some embodiments, the synthetic cytokine receptor is composed of a synthetic gamma chain polypeptide comprising a first dimerization domain, a first transmembrane domain, and an interleukin-2 receptor subunit gamma (IL-2RG) intracellular domain, and a synthetic alpha or beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, an interleukin-21 receptor subunit beta (IL-21RB) intracellular domain and/or an interleukin-9 receptor subunity alpha (IL-9RA) intracellular domain. In some embodiments, the synthetic cytokine receptor is a RACR synthetic cytokine receptor. Exemplary synthetic receptors are described in Section V. Also provided herein are methods of making and/or expanding a population of the engineered cells.

In some embodiments, the population of engineered cells is obtained by introducing a polynucleotide or a vector comprising a polynucleotide encoding a synthetic cytokine receptor into source cells. Source cells can be obtained from primary sources including but not limited to peripheral blood mononuclear cells (PBMCs) or umbilical cord blood (UCB). Sources of cells that can be engineered also can include cells differentiatied from stem cells, such as pluripotent stem cells (iPSCs) which are cells derived from somatic cells (generally fibroblasts or peripheral blood mononuclear cells [PBMCs]), and human embryonic stem cells (hESCs). In some embodiments, the iPSCs are human iPSCs. In some embodiments, the stem cells can be differentiated into other cell types when subjected to appropriate differentiation conditions, and then engineered with a synthetic cytokine receptor. In some embodiments, the differentiation conditions include appropriate differentiation factors for differentiating the cells to lymphoid progenitor cells or lymphocytes, such as ILCs including CILs.

In some embodiments, the engineered cells are ILCs. ILCs are the most recently discovered family of innate immune cells, which are derived from common lymphoid progenitors (CLPs). ILCs can be cytotoxic innate lymphoid (CIL) cells or non-cytotoxic. Among CILs are Natural killer (NK) cells. In some aspects, characteristics that distinguish ILCs from other immune cells include their regular lymphoid morphology, the absence of rearranged antigen receptors found on T cells and B cells, and phenotypic markers usually present on myeloid or dendritic cells (Spits and Cupedo, Annual Review of Immunology. (2012) 20:647-75).

In some aspects, provided herein are pharmaceutical compositions comprising any of the engineered cells or cell populations provided herein (e.g., lymphocyte, such as an ILC, for example a CIL). In even further aspects, provided herein are methods of expanding engineered cells, methods of treating subjects with the engineered cells, and methods of killing or inhibiting cancer cells with the engineered cells. In some aspects, provided herein are kits comprising any of the engineered cells provided herein.

In some embodiments, the cell is engineered from a peripheral blood cell. As used herein, the term “peripheral blood cell” is used to refer to cells that originate from circulating blood and comprise hematopoietic stem cells that are capable of proliferation, selectable differentiation, and maturation. As such, peripheral blood NK cells may alternatively be referred to as differentiated blood-derived NK cells (bdNK).

In some embodiments, the lymphocytes, such as those used to generate engineered CIL cells, may be obtained from a donor or a subject (for autologous therapy) by various means well-known in the art. For example, lymphocytes can be obtained by collecting peripheral blood comprising peripheral blood mononuclear cells (PBMCs) from the patient and subjecting the blood to Ficoll density gradient centrifugation and/or leukapheresis, and then using an isolation kit to isolate a population of lymphocytes from the peripheral blood. In one illustrative embodiment, the population of lymphocytes need not be pure of the selected cell type and may contain other cell types such as T cells, monocytes, macrophages, natural killer cells, and B cells. In some embodiments, the cell population being collected can comprise at least about 90% of the selected cell type, at least about 91%, 92%, 93%, 94%, 95%, 96%, 97%, 98%, 99%, or 100% of the selected cell type.

In some embodiments, NK cells may be generated from NK cell lines. In some embodiments, NK cell lines can be engineered as described herein. In some embodiments, the engineered NK cells include engineered NK cell lines. In some aspects, engineered cell lines allow production of higher amounts of cells without having to expand small numbers of NK cells that are derived from a subject. Engineered cell lines also have the advantage of being well characterized.

In some embodiments, the cell line is a clonal cell line. In some embodiments, the cell line is derived from a patient with NK-cell leukemia or lymphoma. In some embodiments, the NK cell line is NK-92, NK-YS, KHYG-1, NKL, NKG, SNK-6 or IMC-1.

The NK-92 cell line is an NK-like cell line that was initially isolated from the blood of a subject suffering from a large granular lymphoma and subsequently propagated in cell culture. The NK-92 cell line has been described (Gong et al., 1994; Klingemann, 2002). NK-92 cells have a CD3−/CD56+ phenotype that is characteristic of NK cells. They express all of the known NK cell-activating receptors except CD16 but lack all of the known NK cell inhibitory receptors except NKG2A/CD94 and ILT2/LIR1, which are expressed at low levels. Furthermore, NK-92 is a clonal cell line that, unlike the polyclonal NK cells isolated from blood, expresses these receptors in a consistent manner with respect to both type and cell surface concentration. Similarly, NK-92 cells are not immunogenic and do not elicit an immune rejection response when administered therapeutically to a human subject. Indeed NK-92 cells are well tolerated in humans with no known detrimental effects on normal tissues.

Vectors for engineered cell into viral and non-viral vectors. For example, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, episomal vectors, CRISPR-based gene editing, or a method known in the art. In some embodiments, source cells for engineering include: hematopoietic stem cells (HSCs), characterized as being CD34+ and/or CD45+; common lymphoid progenitor cells (CLPs), characterized as being CD45+ CD7+ CD56−; CIL progenitor cells characterized as being CD45+ CD5− CD7+; and/or CIL cells, characterized as being CD45+ CD56+ CD3− and optionally CD5− and/or CD7+.

In some embodiments, source cells may be human embryonic stem cell (hESC) or induced pluripotent stem cell (iPSC) which can be differentiated into a lymphocyte or lymphoid progenitor cell, including any described above. The lymphocyte or lymphoid progenitor cell can then engineered with the synthetic cytokine receptor (e.g. RACR) to drive expansion and/or differentiation in place of normal cytokine pathways from a precursor cell (common lymphoid progenitor or a pre-NK cell). In immunotherapy, source cells be allogeneic or autologous, meaning from a donor or from the subject, respectively.

In some embodiments, CIL cells may be generated from induced pluripotent stem cells (iPSCs). From iPSCs, CIL cells may be derived by sequentially differentiating the iPSCs into hematopoietic progenitor cells (HPCs), also termed hematopoietic stem cells (HSCs); the HPCs into common lymphoid progenitor cells (CLPs); and then the CLPs into CIL cells-termed iPSC-derived cytotoxic innate lymphoid cells (iPSC-CILs or iCIL).

iPSCs are a type of pluripotent stem cell derived from adult somatic cells (e.g., fibroblasts or PBMCs) that have been genetically reprogrammed to an embryonic stem cell-like state through the forced expression of genes and factors important for maintaining the defining properties of embryonic stem cells. iPSCs may be generated from tissues with somatic cells, including, but not limited to, the skin, dental tissue, peripheral blood, and urine. To generate iPSCs, somatic cells may be reprogrammed through methods including, but not limited to, the transient expression of reprogramming factors, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, CRISPR-based gene editing, or recombinant proteins.

While the cell therapy industry has demonstrated the transformative potential of using gene-engineered, patient-derived cells for treating specific disease indications, many challenges remain with using patient-derived materials, including limited expansion capacity and scalability, manufacturing complexity, high cost, variability from patient to patient, and patient access. In contrast, iPSCs are pluripotent stem cells, a type of cell theoretically capable of differentiating into any other cell type-including cytotoxic innate lymphoid (CIL) cells that are applicable to the treatment of cancer. Using iPSCs, it is possible to provide a scalable and simplified manufacturing of a target cell-fighting (e.g., cancer fighting) cell therapy like CIL cells, thus reducing costs and improving patient access to the cell therapies. iPSCs possess an unlimited expansion capacity, meaning they can reproduce and proliferate indefinitely, potentially generating a nearly endless supply of differentiated immune cells for therapy, such as for cancer therapies. iPSCs are also amenable to precision multiplex genome editing, allowing introduction of multiple genetic modifications to enhance their disease targeting capabilities and safety of the immune cells they eventually become. iPSCs can similarly be engineered with the goal of protecting them against allogeneic rejection by the patient's own immune system, improving both their initial expansion and duration of engraftment. Furthermore, while either patient-derived or donor-derived blood materials are inconsistent, iPSCs provide a consistent starting material originating from a single cellular clone, which can permit genomic consistency and integrity in the final cellular product.

Various differentiation protocols for CIL cells are known in the art. Examples of methods for differentiating stem cells (e.g., iPSCs) into multipotent hematopoietic progenitor cells are provided in U.S. Pat. No. 9,624,470, U.S. Patent Appl. No. 2020/0080059, as well as Mesquitta et al., Sci. Rep. 9:6622 (2019), the disclosures of which are incorporated by reference herein in their entireties. Examples of methods for differentiating cells into NK cells are provided in International Patent Application Nos. WO 2013/163171 and WO 2020/124256, U.S. Pat. Nos. 9,260,696, and 10,626,372, as well as Zhu and Kaufman, Methods Mol. Biol. 2048:107-119 (2019) and Matsubara et al., Biochem. Biophys. Res. Commun. 515 (1): 1-8 (2019), the disclosures of which are incorporated by reference herein in their entireties.

Broadly, techniques for differentiating a cell involve modulation of specific cellular pathways, cither directly or indirectly, using polynucleotide-, polypeptide- and/or small molecule-based approaches. The developmental potency of a cell may be modulated, for example, by contacting a cell with one or more modulators. In some embodiments, cells are cultured in the presence of one or more agents to induce cell differentiation (such as, for example, small molecules, proteins, peptides, etc.). In some embodiments, the one or more differentiation agents are introduced to the cell during in vitro culture. The cell may be maintained in the culture medium comprising one or more agents for a period sufficient for the cell to achieve the differentiation phenotype that is desired.

In some embodiments, the stem cells are adapted for feeder-free culture. As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium.

In some embodiments, the culture platform comprises one or more of the following: nutrients, extracts, growth factors, hormones, cytokines and medium additives. Illustrative nutrients and extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Medium additives may include, but are not limited to, MTG, ITS, (ME, antioxidants (for example, ascorbic acid). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFNγ) and other cytokines such as stem cell factor (SCF) and erythropoietin (EPO).

These cytokines may be obtained commercially and may be either natural or recombinant. In some other embodiments, the culture medium of the present disclosure comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (FLT3L); and one or more cytokines from Leukemia inhibitory factor (LIF), IL3, IL6, IL7, IL11, IL15. In some embodiments, the growth factors, mitogens, and cytokines are stage and/or cell type specific in concentrations that are determined empirically or as guided by the established cytokine art. Examples of exogenous cell culture media additives and supplements and cell selection kit components are provided in WO 2020/124256, the disclosure of which is incorporated by reference herein in its entirety.

In some other embodiments, the culture medium of the present disclosure comprises Roswell Park Memorial Institute (RPMI) media, cRPMI1640 media, fetal bovine serum (FBS), Glutamax, Penicillin, streptomycin, Rosuvastatin, BX795, protamine sulfate, brefeldin A, monensin, UM729, IL-2, IL-15, IL-21, IL-18, IL-7, or any combination thereof.

In some embodiments, the method of producing CIL cells of the disclosure comprises: forming embryoid bodies (EBs) comprising aggregates of stem cells; differentiating the cells into hematopoietic stem cells in a first differentiation medium; differentiating the cells into lymphoid progenitor cells in a second differentiation medium; and/or differentiating the cells into differentiated CIL cells in a third differentiation medium.

In some embodiments, the differentiation media contains supplements such as serums, extracts, growth factors, hormones, cytokines and the like.

Differentiation of the cells for generating CIL cells requires a change in a culturing system, such as changing the stimuli agents in the culture medium or the physical state of the cells. A conventional strategy utilizes the formation of embryoid bodies as a common and critical intermediate to initiate the lineage-specific differentiation. Embryoid bodies are aggregates of stem cells that are induced to differentiate by changes in environmental stimuli (e.g., exposure and/or removal of specific molecular/chemical factors; and/or exposure/interaction with three-dimensional structures). Formation of embryoid bodies induces the cells to differentiate cells to a mesoderm specification.

Pluripotent stem cell aggregates are transferred to differentiation medium that provides eliciting cues towards the lineage of choice (e.g., lymphoid). In some embodiments, once EBs have formed, they are dissociated and then cultured in media to induce mesoderm specificity of the cells.

In some aspects of the invention, the embryoid body derivation and differentiation to hematopoietic progenitor cells platforms described above may be carried out under serum-free conditions. Examples of commercially available serum-free media suitable for cell attachment and/or induction include mTeSR™1 or TeSR™2 from Stem Cell Technologies (Vancouver, Canada), Primate ES/iPS cell medium from ReproCELL (Boston, Mass.), StemPro®-34 from Invitrogen (Carlsbad, Calif.), StemPro® hESC SFM from Invitrogen, and X-VIVO™ from Lonza (Basel, Switzerland).

In some embodiments, the media of the induction of mesoderm specificity step comprises one or more of Bone Morphogenic Protein 4 (BMP4), Vascular Endothelial Growth Factor (VEGF), Stem Cell Factor (SCF), Thrombopoietin (TPO), Fms-related Tyrosine Kinase 3 Ligand (FLT3L), basic Fibroblast Growth Factor (bFGF also known as FGF2). In some embodiments, the media of the induction of mesoderm specificity step comprises one or more of Bone Morphogenic Protein 4 (BMP4), Vascular Endothelial Growth Factor (VEGF), Stem Cell Factor (SCF), Thrombopoietin (TPO), Fms-related Tyrosine Kinase 3 Ligand (FLT3L), basic Fibroblast Growth Factor (bFGF also known as FGF2), and/or a ROCK inhibitor.

In some embodiments, the method comprises differentiating the mesoderm specified cells into hematopoietic stem cells in a first differentiation medium.

In some embodiments, the hematopoietic stem cells in a first differentiation media comprises one or more of Bone Morphogenic Protein 4 (BMP4), Fibroblast Growth Factor 2 (FGF2), Vascular Endothelial Growth Factor (VEGF), Stem Cell Factor (SCF), Fms-related Tyrosine Kinase 3 Ligand (FLT3L), Thrombopoietin (TPO), Interleukin-6 (IL-6), Interleukin-3 (IL-3), a TGF-β inhibitor, a PI3K inhibitor, or any combination thereof.

In some embodiments, the TGF-β inhibitor is GW788388. In some embodiments the PI3K inhibitor is LY294002.

In some embodiments, stem cells are differentiated into the mesoderm lineage in a differentiation medium comprising BMP4, FGF2, and VEGF.

In some embodiments, mesoderm cells are differentiated into hematopoietic stem cells in a differentiation medium comprising BMP4, FGF2, VEGF, and SCF.

In some embodiments, hematopoietic stem cells are differentiated into lymphoid progenitor cells in a differentiation medium comprising BMP4, FGF2, VEGF, and SCF.

In some embodiments, lymphoid progenitor cells are differentiated into cytotoxic innate lymphoid cells in a differentiation medium comprising a non-physiological ligand, SCF. FLT3L, and UM729. In some embodiments, the non-physiological ligand is a rapalog. In some embodiments, the non-physiological ligand is rapamycin.

In some embodiments, differentiating the mesoderm specified cells into hematopoietic stem cells in a first differentiation medium occurs for a period of time sufficient for mesoderm specified cells to become CD34+ hematopoietic stem cells.

In some embodiments, the method comprises differentiating the hematopoietic stem cells into lymphoid progenitor cells in a second differentiation medium.

In some embodiments, the lymphoid progenitor cell differentiation media comprises one or more of Bone Morphogenic Protein 4 (BMP4), Fibroblast Growth Factor 2 (FGF2), Vascular Endothelial Growth Factor (VEGF), Stem Cell Factor (SCF), Fms-related Tyrosine Kinase 3 Ligand (FLT3L), Thrombopoietin (TPO), Interleukin-6 (IL-6), Interleukin-3 (IL-3), a TGF-β inhibitor, a PI3K inhibitor, or any combination thereof.

In some embodiments, differentiating the hematopoietic stem cells into lymphoid progenitor cells in a second differentiation medium occurs for a period of time sufficient for hematopoietic stem cells to become Lin-CD34+ CD38−/lo CD45RA+ CD90-lymphoid progenitor cells.

In some embodiments, the first differentiation medium further comprises the non-physiological ligand of the disclosure and/or the second differentiation medium further comprises the non-physiological ligand of the disclosure.

In some embodiments, the first differentiation medium and/or the second differentiation medium is substantially free of at least one cytokine (e.g., IL-2, IL-15, and/or IL-7).

In some embodiments, the differentiation media comprises one or more of Stem Cell Factor (SCF), Interleukin-7 (IL-7), Interleukin-15 (IL-15), Fms-related Tyrosine Kinase 3 Ligand (FLT3L), a Pyrimido-Indole Derivative, or any combination thereof. The Pyrimido-Indole Derivative is UM 729 (pyrimido-[4,5-b]-indole derivative).

In some embodiments, differentiating the lymphoid progenitor cells into CIL cells in a differentiation medium occurs for a period of time sufficient for the lymphoid progenitor cells to become CD3− CD56+ CD45+ CD94+ CD122+/IL-2RB+ CD127/IL-7Rα− FcγRIII/CD16+ KIR+ NKG2A+ NKG2D+ NKp30+ NKp44+ NKP46+ NKp80+. In some embodiments, differentiating the lymphoid progenitor cells into CIL cells in a differentiation medium occurs for a period of time sufficient for the lymphoid progenitor cells to become CD45+ CD56+. In some embodiments, differentiating the lymphoid progenitor cells into CIL cells in a differentiation medium occurs for a period of time sufficient for the lymphoid progenitor cells to become CD45+ CD5− CD7+ CD56+.

In some embodiments, differentiating the lymphoid progenitor cells into CIL cells in a differentiation medium is performed for about 8 to about 18 days. In some embodiments, differentiating the lymphoid progenitor cells into CIL cells in a differentiation medium is performed for about 1, 2, 3, 4, 5, 6, 7, 8, 9, 10, 11, 12, 13, 14, 15, 16, 17, 18, 19, or 20 days.

In some embodiments, the CIL cell differentiation medium is substantially free of Interleukin-15 (IL-15). In some embodiments, the CIL cell differentiation medium is substantially free of Interleukin-7 (IL-7). In some embodiments, the CIL cell differentiation medium is substantially free of Interleukin-2 (IL-2). In some embodiments, the CIL cell differentiation medium is substantially free of IL-15, IL-7, and/or IL-2.

In some embodiments, following differentiation, the CIL cells are expanded in a medium comprising a non-physiological ligand and CD2/NKp46 stimulation. In some embodiments, the CIL cell expansion medium comprises activation beads comprising conjugated anti-CD2 and anti-NKp46 antibodies which stimulate and activate the CIL cells.

In some embodiments, following differentiation, the RACR-iCIL cells are expanded in a medium comprising a non-physiological ligand, membrane-bound IL-21 (mbIL21), and 41BBL K562 initiated feeder cells.

Induced Cytotoxic Innate Lymphoid Cells (iCIL)

In some embodiments, CIL cells may be derived from iPSCs by sequentially differentiating the iPSCs into hematopoietic progenitor cells (HPCs); the HPCs into common lymphoid progenitor cells (CLPs); and then the CLPs into CIL cells-termed “iCIL” cells. In a variation, CIL cells may be derived from HPCs by sequentially differentiating the HPCs into CLPs; and then the CLPs into iCIL cells. In a further variation, CIL cells may be derived by differentiating CLPs into iCIL cells. Engineering of the cells to express the synthetic cytokine receptor may be performed at the HPC, CLP, or iCIL cell step of the differentiation process.

In some embodiments, the iCIL cells are characterized as being CD3− CD56+ CD45+ CD94+ CD122+/IL-2RB+ CD127/IL-7Rα− FcγRIII/CD16+ KIR+ NKG2A+ NKG2D+ NKp30+ NKp44+ NKP46+ NKp80+. In some embodiments, the iCIL cells are characterized by being CD3− CD56+ CD45+ cells. In some embodiments, the iCIL cells are characterized by being CD3− CD56+ cells. In some embodiments, the iCIL cells comprise one or more cell markers selected from the group consisting of CD56+, CD45+, CD94+, CD122+/IL-2RB+, FcγRIII/CD16+, KIR+, NKG2A+, NKG2D+, NKp30+, NKp44+, NKP46+, NKp80+, or any combination thereof. The iCIL cells may be CD45+; CD45+ CD5−; or CD45+ CD5− CD56+.

In some embodiments, the iCIL cells are characterized by being CD45+ CD7+ CD56^{+/lo}.

In some embodiments, after iCIL cells have been transduced with a CAR, the cells are cultured under conditions that promote the activation of the cells.

Culture conditions may be such that the cells can be administered to a patient without concern for reactivity against components of the culture medium. For example, the culture conditions may omit bovine serum products, such as bovine serum albumin. In one illustrative aspect, the activation can be achieved by introducing known activators into the culture medium. In one aspect, the population of iCIL cells can be cultured under conditions promoting activation for about 1 to about 4 days. In one embodiment, the appropriate level of activation can be determined by cell size, proliferation rate, or activation markers determined by flow cytometry. In some embodiments, any of the culturing methods disclosed herein may be used to promote activation of the iCIL cells.

In some embodiments, provided herein is a method of making and/or expanding a population of engineered cells comprising a synthetic cytokine receptor for a non-physiological ligand. In some embodiments, the method comprises:
a. providing engineered cells comprising a synthetic cytokine receptor, optionally by introducing into source cells a polynucleotide encoding a synthetic cytokine receptor, and
b. incubating the engineered cells in media comprising a non-physiological ligand.

In this embodiment, the cytokine receptor comprises a synthetic gamma chain polypeptide comprising a first dimerization domain, a first transmembrane domain, and an interleukin-2 receptor subunit gamma (IL-2RG) intracellular domain, and a synthetic alpha chain or beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, an interleukin-21 receptor subunit beta (IL-21RB) intracellular domain and/or an interleukin-9 receptor subunity alpha (IL-9RB); wherein the non-physiological ligand activates the synthetic cytokine receptor in the engineered cells to induce expansion and/or activation of the engineered cells.

In this embodiment, the cytokine receptor comprises a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; wherein the non-physiological ligand activates the synthetic cytokine receptor in the engineered cells to induce expansion and/or activation of the engineered cells.

In some embodiments, the iPSCs are genetically edited using a lentivirus.

In some embodiments, HSCs are genetically edited using a lentivirus. In some embodiments, blood progenitor cells are genetically edited using a lentivirus. In some embodiments, common lymphoid progenitor cells are genetically edited using a lentivirus. In some embodiments, NK precursor cells are genetically edited using a lentivirus. In some embodiments, common lymphoid progenitor (CLP) cells are genetically edited using a lentivirus.

### III. Retroviral Particles

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an illustrative third-generation system, the Env gene is VSV-G and the promoter is the CMV promoter. The third-generation system uses two packaging plasmids, one encoding gag and pol and the other encoding rev as a further safety feature; an improvement over the single packaging plasmid of so-called second-generation systems. Although safer, the third-generation system can be more cumbersome to use and result in lower viral titers due to the addition of an additional plasmid. Illustrative packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, containing a retroviral or lentiviral envelope including one or more of the env encoded glycoproteins, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the gating adaptor. In some embodiments, a sequence encoding a receptor that specifically binds to the gating adaptor is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to a ligand, and (iv) a 3′ LTR or UTR.

In some embodiments, the retroviral particles comprise a cell surface receptor that binds to a surface marker on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof.

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, innate lymphoid cells, cytotoxic innate lymphoid cells, or NK cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F. Nipah virus (NiV) H and F. Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors. In some embodiments, the vector may be a viral vector, a retroviral vector, a lentiviral vector, a gamma-retroviral vector. In some embodiments, the viral vector comprises a VSV G-protein or functional variant thereof. In some embodiments, the viral vector comprises a Cocal G-protein or functional variant thereof.

### IV. Nucleic Acid Vectors

As used herein, the term “nucleic acid vector” is intended to mean any nucleic acid that functions to carry, harbor or express a nucleic acid of interest. Nucleic acid vectors can have specialized functions such as expression, packaging, pseudotyping, transduction or sequencing, for example. Nucleic acid vectors also can have, for example, manipulatory functions such as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivirus, a foamy virus, and a Sleeping Beauty transposon.

### V. Synthetic Cytokine Receptor Complexes and Systems

In some embodiments, provided herein are synthetic cytokine receptors for engineering cells, including cells that can be used in immunotherapy. In some embodiments, the synthetic cytokine receptor is composed of two polypeptide chains that are able to dimerize to initiate signaling in response to a non-physiological ligand of the synthetic cytokine receptor. In particular embodiments, the synthetic cytokine receptor is a rapamycin activated cytokine receptor (RACR).

Any synthetic receptor system can be used that provides a source of cytokines or a cytokine signal (e.g. JAK/STAT) without having to add exogenous cytokines. In some embodiments, the synthetic cytokine receptor or system induces common cytokine receptor gamma chain signaling. The common cytokine receptor gamma chain is the signaling chain for the receptor complexes of the cytokines IL-2. IL-4, IL-7, IL-9, IL-15 and IL-21. Exemplary examples of cytokines that share the common gamma cytokine chain include, but are not limited to IL-2, IL-7, IL-15, IL-21 and IL-9. Various synthetic cytokine receptors systems are known and can be used, including orthogonal receptors for common gamma chain receptors (e.g. Kalbasi et al. Nature, 2022 607:360; Zhang et al. Sci. Transl. Med., 2021 13 (625): cabg6986), drug-inducible systems of an engineered cytokine fusion with a drug responsive domain (DRD) for controlled or inducible expression of a cytokine, such as described published US20200172879; and synthetic receptors with dimerization domains responsive to a chemical inducer of dimerization (CID).

In some embodiments, the synthetic cytokine receptor contains dimerization domains of a CID ligand. In some embodiments, the synthetic cytokine receptor is a heterodimer of a common cytokine receptor gamma signaling chain and either an alpha or beta cytokine receptor signaling chain, each fused to a dimerization domain. In some embodiments, the dimerization domain can be from an FKBP, a cyclophilin receptor, a steroid receptor, a tetracycline receptor, an estrogen receptor, a glucocorticoid receptor, a vitamin D receptor, Calcineurin A, CyP-Fas, FRB domain of mTOR, GyrB, GAI, GID1, Snap-tag and/or HaloTag, or portions or derivatives thereof. Exemplary of CID and corresponding dimerization domain are known in the art (see, e.g., U.S. Patent Publication No. 2016/0046700, Clackson et al. (1998) Proc Natl Acad Sci USA. 95 (18): 10437-42; Spencer et al. (1993) Science 262 (5136): 1019-24; Farrar et al. (1996) Nature 383 (6596): 178-81; Miyamoto et al. (2012) Nature Chemical Biology 8 (5): 465-70; Erhart et al. (2013) Chemistry and Biology 20 (4): 549-57). In some embodiments, the CID is an estrogen, a glucocorticoid, a vitamin D, a steroid, a tetracycline, a cyclosporine, Rapamycin, Coumermycin, Gibberellin, FK1012, FK506, FKCsA, rimiducid or HaXS, or analogs or derivatives thereof. The dimerization domain may be extracellular (N-terminal to the transmembrane domain) or intracellular. In some embodiments, the CID is a non-physiological ligand or synthetic ligand. In some embodiments, the CID is rapamycin or an analog thereof. Exemplary synthetic receptor dimerization systems are described below.

The non-physiological ligand may activate the synthetic cytokine receptor in the engineered lymphocytes, such as cytotoxic innate lymphoid cells, to induce expansion and/or activation of the engineered cells, such as cytotoxic innate lymphoid cells. In a preferred embodiment, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the engineered lymphocytes (e.g. CIL cells) to induce expansion of the engineered lymphocytes, e.g. CIL cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of lymphocytes, e.g. CIL cells, compared to uninduced cells.

In some embodiments, the lymphocytes, e.g. CIL cells, increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges.

A. Exemplary Synthetic Cytokine Receptors (e.g. RACR)

In some embodiments, the synthetic cytokine receptors of the present disclosure comprise a first transmembrane receptor protein that is a first synthetic cytokine chain (e.g. synthetic gamma chain containing a common receptor gamma signaling chain) and a second transmembrane receptor protein that is a synthetic cytokine chain (e.g. synthetic alpha chain or beta chain containing an intracellular signaling chain of an alpha or beta chain, respectively, of a cytokine receptor), each comprising a dimerization domain. The dimerization domains are able to controllably dimerize in the presence of a non-physiological ligand, thereby activating signaling the synthetic cytokine receptor.

In some embodiments, the first cytokine chain is a synthetic gamma chain polypeptide that comprises a first dimerization domain, a first transmembrane domain, and an interleukin-2 receptor subunit gamma (IL-2RG) intracellular domain. In some embodiments, the second cytokine chain comprises a second dimerization domain, a second transmembrane domain and an intracellular domain of an alpha or beta chain of a cytokine receptor. In some embodiments, the second cytokine chain is a synthetic alpha chain containing an intracellular domain of interleukin-9 receptor subunit alpha (IL-9RA) intracellular domain. In some embodiments, the second cytokine chain is a synthetic beta chain containing an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The dimerization domain may be extracellular (N-terminal to the transmembrane domain) or intracellular (C-terminal to the transmembrane domain and N- or C-terminal to the intracellular domain.

In some embodiments, the synthetic cytokine receptors of the present disclosure comprise a synthetic gamma chain and a synthetic alpha chain, each comprising a dimerization domain. The dimerization domains are able to controllably dimerize in the presence of a non-physiological ligand, thereby activating signaling of the synthetic cytokine receptor. In some embodiments, the synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain. The synthetic alpha chain polypeptide comprises a second dimerization domain, a second transmembrane domain, and an intracellular domain selected from an interleukin-9 receptor subunit alpha (IL-2RA) intracellular domain. The dimerization domain may be extracellular (N-terminal to the transmembrane domain) or intracellular (C-terminal to the transmembrane domain and N- or C-terminal to the IL-9RA intracellular domain).

In some embodiments, the synthetic cytokine receptors of the present disclosure comprise a synthetic gamma chain and a synthetic beta chain, each comprising a dimerization domain. The dimerization domains are able to controllably dimerize in the presence of a non-physiological ligand, thereby activating signaling of the synthetic cytokine receptor. In some embodiments, the synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain. The synthetic beta chain polypeptide comprises a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The dimerization domain may be extracellular (N-terminal to the transmembrane domain) or intracellular (C-terminal to the transmembrane domain and N- or C-terminal to the IL-2RB. IL-7RB or IL-21RB intracellular domain).

In some embodiments, the synthetic gamma chain polypeptide is encoded by a nucleic acid sequence that encodes a signal peptide. In some embodiments, the synthetic alpha chain polypeptide is encoded by a nucleic acid sequence that encodes a signal peptide. In some embodiments, the synthetic beta chain polypeptide is encoded by a nucleic acid sequence that encodes a signal peptide. A skilled artisan is readily familiar with signal peptides that can provide a signal to transport a nascent protein in the cells. Any of a variety of signal peptides can be employed.

In some embodiments, the signal peptide is a CD8a signal sequence shown as SEQ ID NO: 12: MALPVTALLLPLALLLHAARP.

In some embodiments, the signal peptide is a signal sequence shown as SEQ ID NO: 29: MPLGLLWLGLALLGALHAQA.

### 1. Intracellular Domain

In some embodiments, the intracellular signaling domain of the first transmembrane receptor protein comprises an interleukin-2 receptor subunit gamma (IL2Rg) domain. In some embodiments, the IL2Rg domain comprises the sequence set forth in SEQ ID NO: 1. In some embodiments, the IL2Rg Common Gamma Chain Intracellular domain has at least 80% amino acid identity, at least 85% amino acid identity, at least 90% amino acid identity, at least 95% amino acid identity, or 100% amino acid identity to SEQ ID NO: 1.

The sequence of a IL2RG Common Gamma Chain Intracellular domain is set forth in SEQ ID NO: 1:

ERTMPRIPTLKNLEDLVTEYHGNFSAWSGVSKGLAESLQPDYSERLCLVS
EIPPKGGALGEGPGASPCNQHSPYWAPPCYTLKPET.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL2RB) intracellular domain. IL2RB is also known as IL15RB or CD122. Thus, when referred to herein, IL2RB can also mean IL15RB. That is, the terms are used interchangeably in the present disclosure. In some embodiments, the IL2RB intracellular domain comprises the sequence set forth in SEQ ID NO: 2. In some embodiments, the IL2RB intracellular domain has at least 80% amino acid identity, at least 85% amino acid identity, at least 90% amino acid identity, at least 95% amino acid identity, or 100% amino acid identity to SEQ ID NO: 2.

The sequence of a IL2RB intracellular domain is set forth in SEQ ID NO: 2:

NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL7RB) intracellular domain. In some embodiments, the IL7RB intracellular domain comprises the sequence set forth in SEQ ID NO: 3. In some embodiments, the IL7RB intracellular domain has at least 80% amino acid identity, at least 85% amino acid identity, at least 90% amino acid identity, at least 95% amino acid identity, or 100% amino acid identity to SEQ ID NO: 3.

The sequence of a IL7RB intracellular domain is set forth in SEQ ID NO: 3:

KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL21RB) intracellular domain. In some embodiments, the IL21RB intracellular domain comprises the sequence set forth in SEQ ID NO: 4. In some embodiments, the IL21RB intracellular domain has at least 80% amino acid identity, at least 85% amino acid identity, at least 90% amino acid identity, at least 95% amino acid identity, or 100% amino acid identity to SEQ ID NO: 4.

The sequence of a IL21RB intracellular domain is set forth in SEQ ID NO: 4:

SLKTHPLWRLWKKIWAVPSPERFFMPLYKGCSGDFKKWVGAPFTGSSLEL
GPWSPEVPSTLEVYSCHPPRSPAKRLQLTELQEPAELVESDGVPKPSFWP
TAQNSGGSAYSEERDRPYGLVSIDTVTVLDAEGPCTWPCSCEDDGYPALD
LDAGLEPSPGLEDPLLDAGTTVLSCGCVSAGSPGLGGPLGSLLDRLKPPL
ADGEDWAGGLPWGGRSPGGVSESEAGSPLAGLDMDTFDSGFVGSDCSSPV
ECDFTSPGDEGPPRSYLRQWVVIPPPLSSPGPQAS

In some embodiments, the synthetic cytokine receptor comprises a first transmembrane receptor protein comprising an IL-2RG intracellular domain, a first dimerization domain, a second transmembrane receptor protein comprising an IL-9RA intracellular domain, and a second dimerization domain.

In some embodiments, the synthetic alpha chain comprises an interleukin-9 receptor subunit alpha (IL9RA) intracellular domain. In some embodiments, the IL9RA intracellular domain comprises the sequence set forth in SEQ ID NO: 51. In some embodiments, the IL9RA intracellular domain has at least 80% amino acid identity, at least 85% amino acid identity, at least 90% amino acid identity, at least 95% amino acid identity, or 100% amino acid identity to SEQ ID NO: 51.

The sequence of a IL9RA intracellular domain is set forth in SEQ ID NO: 51:

KLSPRVKRIFYQNVPSPAMFFQPLYSVHNGNFQTWMGAHGAGVLLSQDCA
GTPQGALEPCVQEATALLTCGPARPWKSVALEEEQEGPGTRLPGNLSSED
VLPAGCTEWRVQTLAYLPQEDWAPTSLTRPAPPDSEGSRSSSSSSSSNNN
NYCALGCYGGWHLSALPGNTQSSGPIPALACGLSCDHQGLETQQGVAWVL
AGHCQRPGLHEDLQGMLLPSVLSKARSWTF

### 2. Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog. In some embodiments, the FRB domain may comprise a polypeptide sequence at least 80%, at least 90%, at least 95%, at least 96%, at least 97%, at least 98%, at least 99%, or 100% identical to SEQ ID NO: 6 or SEQ ID NO:7. In some embodiments, the FKBP domain may comprise a polypeptide sequence at least 80%, at least 90%, at least 95%, at least 96%, at least 97%, at least 98%, at least 99%, or 100% identical to SEQ ID NO: 5. In some embodiments, the FKBP domain may comprise a polypeptide sequence at least 80%, at least 90%, at least 95%, at least 96%, at least 97%, at least 98%, at least 99%, or 100% identical to SEQ ID NO: 48. In some embodiments, The FKBP domain may comprise a polypeptide sequence at least 80%, at least 90%, at least 95%, at least 96%, at least 97%, at least 98%, at least 99%, or 100% identical to SEQ ID NO: 30. In some embodiments, The FKBP domain may comprise a polypeptide sequence at least 80%, at least 90%, at least 95%, at least 96%, at least 97%, at least 98%, at least 99%, or 100% identical to SEQ ID NO: 49.

In some embodiments, the sequence of an illustrative FKBP domain is set forth in SEQ ID NO: 5:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

In some embodiments, the sequence of an illustrative FKBP domain is set forth in SEQ ID NO: 48:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKL

In some embodiments, the sequence of an illustrative FKBP domain is set forth in SEQ ID NO: 30:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLGE

In some embodiments, the sequence of an illustrative FKBP domain is set forth in SEQ ID NO: 49:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLGE

In some embodiments, the sequence of an illustrative FRB domain is set forth in SEQ ID NO: 6:

ILWHEMWHEGLEEASRLYFGERNVKGMFEVLEPLHAMMERGPQTLKETSF
NQAYGRDLMEAQEWCRKYMKSGNVKDLTQAWDLYYHVFRRISK

In some embodiments, the sequence of variant FRB domain (FRB mutant domain) is set forth in SEQ ID NO: 7:

ILWHEMWHEGLEEASRLYFGERNVKGMFEVLEPLHAMMERGPQTLKETSF
NQAYGRDLMEAQEWCRKYMKSGNVKDLLQAWDLYYHVFRRISK

In some embodiments, the first dimerization domain is set forth in SEQ ID NO: 5 and the second dimerization domain is set forth in SEQ ID NO:6.

In some embodiments, the first dimerization domain is set forth in SEQ ID NO: 48 and the second dimerization domain is set forth in SEQ ID NO:6.

In some embodiments, the first dimerization domain is set forth in SEQ ID NO: 30 and the second dimerization domain is set forth in SEQ ID NO:6.

In some embodiments, the first dimerization domain is set forth in SEQ ID NO: 5 and the second dimerization domain is set forth in SEQ ID NO:7.

In some embodiments, the first dimerization domain is set forth in SEQ ID NO: 48 and the second dimerization domain is set forth in SEQ ID NO:7.

In some embodiments, the first dimerization domain is set forth in SEQ ID NO: 30 and the second dimerization domain is set forth in SEQ ID NO:7.

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) ii) cyclophiliA (CypA); or
iii) iii) gyrase B (CyrB);
with the corresponding non-physiological ligands being, respectively
i) FK1012, AP1510, AP1903, or AP20187;
ii) ii) cyclosporin-A (CsA); or
iii) iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

### 3. Transmembrane Domains

The transmembrane domain is the sequence of the synthetic cytokine receptor that spans the membrane. The transmembrane domain may comprise a hydrophobic alpha helix. In some embodiments, the transmembrane domain is derived from a human protein.

The sequence of a transmembrane (TM) domain is shown as SEQ ID NO: 8: VVISVGSMGLIISLLCVYFWL

The sequence of a TM domain is shown as SEQ ID NO: 9:

VAVAGCVFLLISVLLLSGL

The sequence of TM domain is shown as SEQ ID NO: 10:

PILLTISILSFFSVALLVILACVLW

The sequence of a TM domain is shown as SEQ ID NO: 11:

GWNPHLLLLLLLVIVFIPAFW

The sequence of a TM domain is shown as SEQ ID NO: 36:

IPWLGHLLVGLSGAFGFIILVYLLI.

In some embodiments, the TM domain and the intracellular signaling domain are from the same cytokine receptor. In some embodiments, the synthetic gamma chain polypeptide contains an IL-2RG TM domain and an IL-2RG intracellular domain. In some embodiments, the synthetic beta chain polypeptide contains an IL-2RB TM domain and an IL-2RB intracellular domain. In some embodiments, the synthetic beta chain polypeptide contains an IL-7RB TM domain and an IL-7RB intracellular domain. In some embodiments, the synthetic beta chain polypeptide contains an IL-21RB TM domain and an IL-21RB intracellular domain. In some embodiments, the synthetic alpha chain polypeptide contains an IL-9RA TM domain and an IL-9RA intracellular domain.

In some embodiments, one or more additional contiguous amino acids of the ectodomain directly adjacent to the TM domain of the cytokine receptor also can be included as part of the polypeptide sequence of a chain of the synthetic cytokine receptor. In some embodiments, 1-20 contiguous amino acids of the ectodomain adjacent to the TM domain of the cytokine receptor is included as part of the polypeptide sequence of a chain of the synthetic cytokine receptor. The portion of the ectodomain may be a contiguous sequence of 1, 2, 3, 4, 5, 6, 7, 8, 9, 10, 11, 12, 13, 14, 15, 16, 17, 18, 19 or 20 amino acids directly adjacent (e.g., N-terminal to) the TM sequence.

In some embodiments, the synthetic cytokine receptor is composed of a synthetic gamma chain polypeptide that contains a FKBP12 dimerization domain, an IL-2RG transmembrane domain and an IL-2RG intracellular domain, and a synthetic beta chain polypeptide that contains an FRB dimerization domain, an IL-2RB transmembrane domain and an IL-2RB intracellular domain. In some embodiments, the synthetic cytokine receptor is composed of a synthetic gamma chain polypeptide that contains a FRB dimerization domain, an IL-2RG transmembrane domain and an IL-2RG intracellular domain, and a synthetic beta chain polypeptide that contains an FKBP12 dimerization domain, an IL-2RB transmembrane domain and an IL-2RB intracellular domain.

In some embodiments, the synthetic gamma chain polypeptide contains an IL-2RG TM domain comprising the sequence set forth in SEQ ID NO: 8 and an IL-2RG intracellular domain comprising the sequence set forth in SEQ ID NO:1. In some embodiments, the synthetic gamma chain polypeptide contains an IL-2RG TM domain comprising the sequence set forth in SEQ ID NO: 31 and an IL-2RG intracellular domain comprising the sequence set forth in SEQ ID NO:1.

In some embodiments, the synthetic beta chain polypeptide contains an IL-2RB TM domain comprising the sequence set forth in SEQ ID NO: 36 and an IL-2RB intracellular domain comprising the sequence set forth in SEQ ID NO:2. In some embodiments, the synthetic beta chain polypeptide contains an IL-2RB TM domain comprising the sequence set forth in SEQ ID NO: 35 and an IL-2RB intracellular domain comprising the sequence set forth in SEQ ID NO:2.

In some embodiments, the synthetic cytokine receptor is composed of a synthetic gamma chain polypeptide that contains a FKBP12 dimerization domain and an IL-2RG intracellular domain, and a synthetic beta chain polypeptide that contains an FRB dimerization domain and an IL-2RB intracellular domain. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28. In some embodiments, the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

In some embodiments, the synthetic cytokine receptor is composed of a synthetic gamma chain polypeptide that contains a FKBP12 dimerization domain and an IL-2RG intracellular domain, and a synthetic beta chain polypeptide that contains an FRB dimerization domain and an IL-2RB intracellular domain. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28. In some embodiments, the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

In some embodiments, the synthetic cytokine receptor is composed of a synthetic gamma chain polypeptide set forth in SEQ ID NO:28 and a synthetic beta chain polypeptide set forth in SEQ ID NO:33.

In some embodiments, the synthetic cytokine receptor is composed of a synthetic gamma chain polypeptide that contains a FKBP12 dimerization domain, an IL-2RG transmembrane domain and an IL-2RG intracellular domain, and a synthetic beta chain polypeptide that contains an FRB dimerization domain, an IL-7RB transmembrane domain and an IL-7RB intracellular domain. In some embodiments, the synthetic cytokine receptor is composed of a synthetic gamma chain polypeptide that contains a FRB dimerization domain, an IL-2RG transmembrane domain and an IL-2RG intracellular domain, and a synthetic beta chain polypeptide that contains an FKBP12 dimerization domain, an IL-7RB transmembrane domain and an IL-7RB intracellular domain.

In some embodiments, the synthetic cytokine receptor is composed of a synthetic gamma chain polypeptide that contains a FKBP12 dimerization domain, an IL-2RG transmembrane domain and an IL-2RG intracellular domain, and a synthetic beta chain polypeptide that contains an FRB dimerization domain, an IL-21RB transmembrane domain and an IL-21RB intracellular domain. In some embodiments, the synthetic cytokine receptor is composed of a synthetic gamma chain polypeptide that contains a FRB dimerization domain, an IL-2RG transmembrane domain and an IL-2RG intracellular domain, and a synthetic beta chain polypeptide that contains an FKBP12 dimerization domain, an IL-21RB transmembrane domain and an IL-21RB intracellular domain.

In some embodiments, the synthetic cytokine receptor is composed of a synthetic gamma chain polypeptide that contains a FKBP12 dimerization domain, an IL-2RG transmembrane domain and an IL-2RG intracellular domain, and a synthetic beta chain polypeptide that contains an FRB dimerization domain, an IL-9RA transmembrane domain and an IL-9RA intracellular domain. In some embodiments, the synthetic cytokine receptor is composed of a synthetic gamma chain polypeptide that contains a FRB dimerization domain, an IL-2RG transmembrane domain and an IL-2RG intracellular domain, and a synthetic beta chain polypeptide that contains an FKBP12 dimerization domain, an IL-9RA transmembrane domain and an IL-9RA intracellular domain.

In some embodiments, the synthetic cytokine receptor is able to be bound by the non-physiological ligand rapamycin or a rapamycin analog. In some embodiments, the synthetic cytokine receptor is responsive to the non-physiological ligand rapamycin or a rapamycin analog, in which binding of the non-physiological ligand to the dimerization domains of the synthetic cytokine receptor induces cytokine receptor-mediated signaling in the cell, such as via the JAK/STAT pathway.

### 4. Non-Physiological Ligand

In various embodiments of the compositions and methods of the disclosure, the system comprises a non-physiological ligand. Illustrative small molecules useful as ligands include, without limitation: rapamycin, fluorescein, fluorescein isothiocyanate (FITC), 4-[(6-methylpyrazin-2-yl)oxy]benzoic acid (aMPOB), folate, rhodamine, acetazolamide, and a CA9 ligand.

In some embodiments, the synthetic cytokine receptor is activated by a ligand. In some embodiments, the ligand is a non-physiological ligand.

In some embodiments, the non-physiological ligand is a rapalog.

In some embodiments, the non-physiological ligand is rapamycin.

In some embodiments, the non-physiological ligand is AP21967.

In some embodiments, the non-physiological ligand is FK506.

In some embodiments, the non-physiological ligand is FK1012. In some embodiments, the non-physiological ligand is AP1510. In some embodiments, the non-physiological ligand is AP1903. In some embodiments, the non-physiological ligand is AP20187. In some embodiments, the non-physiological ligand is cyclosporin-A (CsA). In some embodiments, the non-physiological ligand is coumermycin.

In some embodiments, the synthetic cytokine receptor complex activated by folate, fluorescein, aMPOB, acetazolamide, a CA9 ligand, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the non-physiological ligand may be an inorganic or organic compound that is less than 1000 Daltons.

In some embodiments, the ligand may be rapamycin or a rapamycin analog (rapalog). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

Thus, in some embodiments, the rapalog is everolimus, novolimus, pimecrolimus, ridaforolimus, tacrolimus, temsirolimus, umirolimus, zotarolimus, Temsirolimus (CCI-779), C20-methallylrapamycin, C16-(S)-3-methylindolerapamycin, C16-(S)-3-methylindolerapamycin (C16-iRap), AP21967 (A/C Heterodimerizer, Takara Bio®), sodium mycophenolic acid, benidipine hydrochloride, rapamine, AP23573 (Ridaforolimus), AP1903 (Rimiducid), or metabolites, derivatives, and/or combinations thereof.

In some embodiments, the ligand comprises FK1012 (a semisynthetic dimer of FK506), tacrolimus (FK506), FKCsA (a composite of FK506 and cyclosporine), rapamycin, coumermycin, gibberellin, HaXS dimerizer (chemical dimerizers of HaloTag and SNAP-tag), TMP-HTag (trimethoprim haloenzyme protein dimerizer), or ABT-737 or functional derivatives thereof.

In some embodiments, the non-physiological ligand is present or provided in an amount from 0 nM to 1000 nM such as, e.g., 0.05 nM, 0.1 nM, 0.5. nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, or 1000 nM, or an amount that is within a range defined by any two of the aforementioned amounts.

In some embodiments, the non-physiological ligand is AP21967 and is present or provided at 10 nM. In some embodiments, the non-physiological ligand is AP21967 and is present or provided at 20 nM. In some embodiments, the non-physiological ligand is AP21967 and is present or provided at 50 nM. In some embodiments, the non-physiological ligand is AP21967 and is present or provided at 100 nM.

In some embodiments, the non-physiological ligand is rapamycin and is present or provided at 1 nM. In some embodiments, the non-physiological ligand is rapamycin and is present or provided at 10 nM. In some embodiments, the non-physiological ligand is rapamycin and is present or provided at 20 nM. In some embodiments, the non-physiological ligand is rapamycin and is present or provided at 50 nM.

In some embodiments, the non-physiological ligand is a rapalog and is present or provided at 1 nM. In some embodiments, the non-physiological ligand is a rapalog and is present or provided at 10 nM. In some embodiments, the non-physiological ligand is a rapalog and is present or provided at 20 nM. In some embodiments, the non-physiological ligand is a rapalog and is present or provided at 50 nM. In some embodiments, the non-physiological ligand is a rapalog and is present or provided at 100 nM.

In some embodiments, the non-physiological ligand is present or provided at 1 nM.

In some embodiments, the non-physiological ligand is present or provided at 10 nM.

In some embodiments, the non-physiological ligand is present or provided at 100 nM.

In some embodiments, the non-physiological ligand is present or provided at 1000 nM.

### B. Cytosolic FRB

In some embodiments, the engineered cells, such as lymphocytes or progenitors thereof, including iCIL cells, can be contacted with free cytosolic FRB (i.e., soluble FRB). As described in more detail elsewhere herein, rapamycin normally binds to FKBP12, and the FKBP12-rapamycin complex then binds to the FRB subunit of mTOR and blocks mTOR signaling. Thus, contacting a cell with rapamycin could, in some cases, inhibit or reduce cell growth and expansion. In contrast to expansion of NK cells, certain drugs are known to have a suppressive effect on NK cells. The small-molecule rapamycin has been reported to suppress NK cell function by Wai et al. Transplantation. 85 (1): 145-149 (2008). In some embodiments, the cells can be made “rapamycin resistant” by providing free cytosolic FRB to the cell in order to complex with rapamycin and thereby eliminate or reduce rapamycin-mediated growth inhibition of a source cell or of an engineered cell, such as an engineered lymphocyte for example an iCIL.

In some embodiments, soluble FRB can be microinjected into a cell as described, including a cell engineered with a synthetic cytokine receptor, such as a lymphocyte, including a CIL (e.g. NK cell) to eliminate or reduce rapamycin-mediated growth inhibition. In some embodiments, a cell, such as a CIL or NK cell, can be transduced with a vector containing soluble FRB to eliminate or reduce rapamycin-mediated growth inhibition. In some embodiments, soluble FRB can be added to cell culture media to eliminate or reduce rapamycin mediated growth inhibition.

In an embodiment where soluble FRB is microinjected into a stem cell or NK cell, the soluble FRB is injected at a concentration of 4 mg/mL, 4.5 mg/mL, 5 mg/mL, 5.5 mg/mL, or 6 mg/mL. In an embodiment where soluble FRB is microinjected into a cell, such as a CIL or NK cell, the soluble FRB is injected at a concentration of 1 μM.

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the transduced cells and promote consistent activation of transduced cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. The cytosolic FRB domain may comprise a polypeptide sequence at least 80%, at least 90%, at least 95%, at least 96%, at least 97%, at least 98%, at least 99%, or 100% identical to SEQ ID NO: 6 or SEQ ID NO: 7. FRB domain may be a naked FRB domain consisting essentially of a polypeptide having a polypeptide sequence at least 80%, at least 90%, at least 95%, at least 96%, at least 97%, at least 98%, at least 99%, or 100% identical to SEQ ID NO: 6 or SEQ ID NO: 7. Advantageously, the cytosolic FRB confers resistance to the immunosuppressive effect of the non-physiological ligand (e.g., rapamycin or rapalog). In some embodiments, the cytosolic FRB domain may comprise a polypeptide sequence at least 80%, at least 90%, at least 95%, at least 96%, at least 97%, at least 98%, at least 99%, or 100% identical to SEQ ID NO: 50. In some embodiments, the FRB domain may be a naked FRB domain consisting essentially of a polypeptide having a polypeptide sequence at least 80%, at least 90%, at least 95%, at least 96%, at least 97%, at least 98%, at least 99%, or 100% identical to SEQ ID NO: 50.

In some embodiments, the cells are contacted with an FRB domain protein that has the sequence set forth in SEQ ID NO:6.

In some embodiments, the cells are contacted with an FRB domain protein that has the sequence set forth in SEQ ID NO:7.

In some embodiments, the cells are contacted with an FRB domain protein that has the sequence set forth in SEQ ID NO:50.

### VI. Gene Editing

In some embodiments the cells described herein (e.g., iPSCs or CILs) may be modified by gene editing. In some embodiments, the gene edited iPSCs as described may be used as source cells for differentiation into CILs.

Genome editing generally refers to the process of editing or changing the nucleotide sequence of a genome, preferably in a precise, desirable and/or pre-determined manner. Examples of compositions, systems, and methods of genome editing described herein use site-directed nucleases to cut or cleave DNA at precise target locations in the genome, thereby creating a double-strand break (DSB) in the DNA. Such breaks can be repaired by endogenous DNA repair pathways, such as homology directed repair (HDR) and/or non-homologous end-joining (NHEJ) repair (see e.g., Cox et al., (2015) Nature Medicine 21 (2): 121-31).

In some embodiments, the cells described herein (e.g., stem cells, CILs) are genetically modified. In some embodiments, the modification involves knocking out one or more endogenous genes using a DNA-targeted protein and a nuclease or an RNA-guided nuclease and/or knocking in one or more exogenous genes of interest. In some embodiments, a gene of interest is knocked into a particular locus of interest. In some embodiments, the gene of interest is a synthetic cytokine receptor complex. In some embodiments, the synthetic cytokine receptor complex is activated by rapamycin. In some embodiments, the synthetic cytokine receptor complex is a rapamycin activated cytokine receptor (RACR). In some embodiments, a RACR is knocked into a locus of interest. In some embodiments, the gene of interest is a chimeric antigen receptor.

In some embodiments, the modification comprises contacting a cell with a DNA-targeted protein and a nuclease or an RNA-guided nuclease. In some embodiments, a DNA-targeted protein and a nuclease or an RNA-guided nuclease includes zinc finger protein (ZFP), a clustered regularly interspaced short palindromic nucleic acid (CRISPR), or a TAL-effector nuclease (TALEN). In some embodiments, CRISPR-Cas9 is used. In some embodiments, CRISPR-Mad7 is used.

Rejection of cellular therapeutics (e.g., CAR T cells) is due at least to mismatches of human leukocyte antigen (HLA) between donor and recipient. One solution recently identified is disrupting expression of genes involved in this rejection, such as T cell receptor alpha constant (TRAC), beta-2-microglobulin (B2M), and signal regulatory protein alpha (SIRPA). Accordingly, in some embodiments, the cells described herein (e.g., iPSCs, CILs), are genetically engineered to knockout a B2M locus, a TRAC locus, and/or a SIRPA locus. In some embodiments, the cells described herein are genetically engineered to knockout a B2M locus. In some embodiments, the cells described herein are genetically engineered to knockout a TRAC locus. In some embodiments, the cells described herein are genetically engineered to knockout a SIRPA locus.

In some embodiments, the cells described herein are genetically engineered to be rapamycin resistant. Rapamycin is small molecule drug that inhibits the mTOR pathway, which is a pathway that is essential for cell growth and expansion. Thus, contacting a cell with rapamycin could, in some cases, inhibit or reduce cell growth and expansion. In some embodiments, to eliminate or reduce rapamycin-mediated growth inhibition of a source cell or CIL using the provided methods, the provided cells are disrupted in an endogenous gene involved in rapamycin function, thereby rendering such cells “rapamycin resistant.” It is understood that reference to a “rapamycin resistant” cell refers to the ability of a cell's endogenous mTOR pathway not to be affected or impacted by the presence of rapamycin or a rapamycin analog. However, it is further understood that a “rapamycin resistant” cell may nevertheless be responsive to rapamycin via a pathway that does not involve mTOR, such as due to activation of a synthetic RACR as described herein.

In some embodiments, the cells are genetically engineered to disrupt a gene associated with rapamycin recognition. In some embodiments, the cells are genetically engineered to disrupt the mTOR gene. In some embodiments, the mTOR gene is FKBP-12 (also known as FKBP-1A, FKBP1, FKBP12, PKC12, PKCI2, PPIASE). FKBP12 is an essential binder of rapamycin and required for its function. In some embodiments, the cells are genetically engineered to disrupt the FKBP12 gene. In some embodiments, the cells are genetically engineered to knockout the FKB12 gene to induce rapamycin resistance. In some embodiments, the disruption of the endogenous FKBP12 gene of the source stem cell (e.g. iPSC) is through genetic knock out with CRISPR-Cas system. In a normal cell without a genetic disruption of FKBP12, FKBP12 is the primary binder of rapamycin, and the FKBP12-rapamycin complex then binds to the FRB subunit of mTOR and blocks mTOR signaling. By disrupting expression of the FKBP12 gene such as by FKBP12 knockout, results herein demonstrate successful rapamycin suppression activity because rapamycin has no function without first complexing with FKBP1A. Thus, genetic disruption of FKBP12, such as by gene knock out, renders the stem cells (e.g. iPSCs) highly resistant to rapamycin-mediated mTOR inhibition, enabling robust growth of the stem cells (e.g. iPSC) even in the presence of high doses of rapamycin. In some embodiments, the ability to render cells resistant to rapamycin growth suppression permits engagement of the RACR by rapamycin during cell differentiation without deleterious effects. Further, knock out of FKBP12 avoids competition of FKBP12 with the RACR for binding to rapamycin. Thus, in some embodiments, the ability to render cells resistant to rapamycin growth by FKBP12 knock out also permits activation of RACR-containing cells in vivo and suppresses potential allogeneic anti-graft responses through mTOR suppression of the host immune system.

In some embodiments, the cells described herein are genetically engineered to comprise a nucleotide sequence encoding a synthetic cytokine receptor in an endogenous gene. In some embodiments, the synthetic cytokine receptor is engineered into a gene such that expression of the endogenous gene is not disrupted. In some embodiments, the synthetic cytokine receptor is engineered into a safe-harbor locus.

In some embodiments, the cells described herein genetically engineered to comprise a nucleotide sequence encoding a synthetic cytokine receptor in a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB).

In some embodiments, the gene of interest inserted into an endogenous locus is a synthetic cytokine receptor complex. In some embodiments, the endogenous promoter of the particular locus is used. In some embodiments, additional promoter(s) may be included such that two or more promoters drive expression of the exogenous gene of interest.

In some embodiments, the cells described herein are genetically engineered to comprise a nucleotide sequence encoding a synthetic cytokine receptor complex in a disrupted gene. For example, in some embodiments the cells comprise a disrupted B2M gene and a nucleotide sequence encoding the synthetic cytokine receptor in the disrupted B2M gene.

In some embodiments, the cells described herein (e.g., iPSCs, CILs) comprise (i) a disrupted B2M locus, and (ii) a nucleotide sequence encoding a synthetic cytokine receptor complex (e.g., a RACR) under control of the endogenous B2M promoter and an EEF1A promoter.

In some embodiments, the cells described herein (e.g., iPSCs, CILs) comprise (i) a disrupted B2M locus, and (ii) a nucleotide sequence encoding a synthetic cytokine receptor complex (e.g., a RACR) inserted into the endogenous B2M gene and under control of the endogenous B2M promoter and an EEF1A promoter.

In some embodiments, cells comprising (i) a disrupted B2M locus and (ii) a nucleotide sequence encoding a synthetic cytokine receptor complex (e.g., a RACR) are produced by any of the methods described below.

### A. Systems for Genome Editing

In some embodiments, a system for editing a cell described herein comprises a site-directed nuclease, such as a CRISPR/Cas system and optionally a gRNA. In some embodiments, the system comprises an engineered nuclease. In some embodiments, the system comprises a site-directed nuclease. In some embodiments, the site-directed nuclease comprises a CRISPR/Cas nuclease system. In some embodiments, the Cas nuclease is Cas9. In some embodiments, the nuclease is Mad7. In some embodiments, the guide RNA comprising the CRISPR/Cas system is a single guide RNA (sgRNA).

### 1. CRISPR/Cas Nuclease Systems

Naturally-occurring CRISPR/Cas systems are genetic defense systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et al., (2017) Curr Opin Microbiol 37:67-78).

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g., Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g., genes).

In some embodiments, a system for editing a cell described herein comprises a nuclease capable of inducing a DNA break within an endogenous target gene in the cell. In some embodiments, the DNA break comprises a double stranded break (DSB), which is induced by a nuclease capable of inducing a DSB by cleaving both strands of double stranded DNA at a cleavage site. In some embodiments, the DNA break comprises a single strand break (SSB) at a cleavage site in the sense strand or the antisense strand of the endogenous target gene. In some embodiments, the DNA break comprises a SSB at a cleavage site in the sense strand, and a SSB at a cleavage site in the antisense strand, thereby resulting in a DSB. In some embodiments, the DSB is induced by a pair of recombinant nucleases, e.g., nickases, that are each capable of inducing a single strand break (SSB) in opposite DNA strands at different cleavage sites, e.g., at a cleavage site upstream of the gene variant in one strand and at a cleavage site downstream of the gene variant in the other strand of the target gene. In some embodiments, a first of the pair of nickases forms a complex with a first guide RNA, e.g., a first sgRNA, for targeting cleavage to one strand, e.g., the sense strand, and the second of the pair of nickases forms a complex with a second guide RNA, e.g., a second sgRNA, for targeting cleavage to the other strand, e.g., the antisense strand. In some embodiments, a DSB is induced through a SSB on each of the opposite strands, i.e., the sense strand and the antisense strand, of an endogenous target gene in the cell.

In general, genes are located in double stranded DNA that includes a sense strand and an antisense strand, which are complementary to one another. The sense strand is also referred to as the coding strand because its sequence is the DNA version of the RNA sequence that is transcribed. The antisense strand is also referred to as the template strand because its sequence is complementary to the RNA sequence that is transcribed.

i. Guide RNAs (gRNAs)

Engineered CRISPR/Cas systems comprise at least two components: 1) a guide RNA (gRNA) molecule and 2) a Cas nuclease, which interact to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g., a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA: tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the target nucleic acid (e.g., endogenous gene) is B2M. In some embodiments, the crRNA comprises the nucleotide sequence set forth in SEQ ID NO:18, or a nucleotide sequence having at least 80%, 85%, 90%, or 95% sequence identity to the nucleotide sequence set forth in SEQ ID NO:18. In some embodiments, the crRNA comprises the nucleotide sequence set forth in SEQ ID NO: 18.

In some embodiments, the target nucleic acid (e.g., endogenous gene) is FKBP12. In some embodiments, the crRNA comprises the nucleotide sequence set forth in any one of SEQ ID NOS: 19, 20, and 21, or a nucleotide sequence having at least 80%, 85%, 90%, or 95% sequence identity to a nucleotide sequence set forth in any one of SEQ ID NOS: 19, 20, and 21. In some embodiments, the crRNA comprises the nucleotide sequence set forth in any one of SEQ ID NOS: 19, 20, and 21. In some embodiments, the crRNA comprises the nucleotide sequence set forth in SEQ ID NO: 19, or a nucleotide sequence having at least 80%, 85%, 90%, or 95% sequence identity to the nucleotide sequence set forth in SEQ ID NO: 19. In some embodiments, the crRNA comprises the nucleotide sequence set forth in SEQ ID NO: 19 In some embodiments, the crRNA comprises the nucleotide sequence set forth in SEQ ID NO:20, or a nucleotide sequence having at least 80%, 85%, 90%, or 95% sequence identity to the nucleotide sequence set forth in SEQ ID NO:20. In some embodiments, the crRNA comprises the nucleotide sequence set forth in SEQ ID NO: 20. In some embodiments, the crRNA comprises the nucleotide sequence set forth in SEQ ID NO:21, or a nucleotide sequence having at least 80%, 85%, 90%, or 95% sequence identity to the nucleotide sequence set forth in SEQ ID NO:21. In some embodiments, the crRNA comprises the nucleotide sequence set forth in SEQ ID NO: 21.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

a. Single Guide RNA (sgRNA)

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can be unmodified or modified. For example, modified sgRNAs can comprise one or more 2′-O-methyl phosphorothioate nucleotides.

By way of illustration, guide RNAs used in the CRISPR/Cas system, or other smaller RNAs can be readily synthesized by chemical means, as illustrated herein and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

b. Spacers

In some embodiments, the gRNAs comprise a spacer sequence. A spacer sequence is a sequence that defines the target site of a target nucleic acid (e.g. DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. In some embodiments, a spacer sequence corresponding to a target sequence adjacent to a PAM sequence is complementary to the non-PAM strand of the target nucleic acid. Thus, in some embodiments, a spacer sequence which corresponds to a target sequence adjacent to a PAM sequence is identical to the PAM strand. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

In some embodiments, the spacer comprise at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

ii. Methods of Making gRNAs

Methods for making gRNAs are known to those of skill in the art and include but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some embodiments, non-natural modified nucleobases are introduced into polynucleotides, e.g., gRNA, during synthesis or post-synthesis. In certain embodiments, modifications are on internucleoside linkages, purine or pyrimidine bases, or sugar. In some embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1 (3), 165-187 (1990).

In some embodiments, the disclosure provides nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g.: spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR-Cas system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex. In some embodiments, the CRISPR/Cas complex is an engineered Class 2 Type V CRISPR system. In some embodiments, the endonuclease is Mad7.

iii. Cas Nuclease

In some embodiments, the disclosure provides compositions and systems (e.g., an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I. Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13 (11): 722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein are from Streptococcus thermophilus (StCas9). In some embodiments, the Cas9 protein are from Neisseria meningitides (NmCas9). In some embodiments, the Cas9 protein are from Staphylococcus aureus (SaCas9). In some embodiments, the Cas9 protein are from Campylobacter jejuni (CjCas9).

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

In some embodiments, the Cas nuclease is a Mad endonuclease. CRISPR/Mad systems are closely related to the Type V (Cpf1-like) of Class-2 family of Cas enzymes. In some embodiments, the CRISPR-Mad system employs an Eubacterium rectale Mad7 endonuclease or variant thereof. The Mad7-crRNA complex cleaves target DNA by identification of a PAM 5′-YTTN.

### 2. Engineered Nucleases

In some embodiments, the cells described herein are genetically engineered with a site-directed nuclease, wherein the site-directed nuclease is an engineered nuclease. Exemplary engineered nucleases are meganuclease (e.g., homing endonucleases), ZFN, TALEN, and megaTAL.

Naturally-occurring meganucleases may recognize and cleave double-stranded DNA sequences of about 12 to 40 base pairs and are commonly grouped into five families. In some embodiments, the meganuclease are chosen from the LAGLIDADG family, the GIY-YIG family, the HNH family, the His-Cys box family, and the PD−(D/E)XK family. In some embodiments, the DNA binding domain of the meganuclease are engineered to recognize and bind to a sequence other than its cognate target sequence. In some embodiments, the DNA binding domain of the meganuclease are fused to a heterologous nuclease domain. In some embodiments, the meganuclease, such as a homing endonuclease, are fused to TAL modules to create a hybrid protein, such as a “megaTAL” protein. The megaTAL protein have improved DNA targeting specificity by recognizing the target sequences of both the DNA binding domain of the meganuclease and the TAL modules.

ZFNs are fusion proteins comprising a zinc-finger DNA binding domain (“zinc fingers” or “ZFs”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats are combined to recognize longer DNA sequences, such as, e.g., 9-, 12-, 15-, or 18-bp, etc. In some embodiments, the ZFN comprise ZFs fused to a nuclease domain from a restriction endonuclease. For example, the restriction endonuclease is FokI. In some embodiments, the nuclease domain comprises a dimerization domain, such as when the nuclease dimerizes to be active, and a pair of ZFNs comprising the ZF repeats and the nuclease domain is designed for targeting a target sequence, which comprises two half target sequences recognized by each ZF repeats on opposite strands of the DNA molecule, with an interconnecting sequence in between (which is sometimes called a spacer in the literature). For example, the interconnecting sequence is 5 to 7 bp in length. When both ZFNs of the pair bind, the nuclease domain may dimerize and introduce a DSB within the interconnecting sequence. In some embodiments, the dimerization domain of the nuclease domain comprises a knob-into-hole motif to promote dimerization. For example, the ZFN comprises a knob-into-hole motif in the dimerization domain of FokI.

The DNA binding domain of TALENs usually comprises a variable number of 34 or 35 amino acid repeats (“modules” or “TAL modules”), with each module binding to a single DNA base pair, A. T. G, or C. Adjacent residues at positions 12 and 13 (the “repeat-variable di-residue” or RVD) of each module specify the single DNA base pair that the module binds to. Though modules used to recognize G may also have affinity for A. TALENs benefit from a simple code of recognition-one module for each of the 4 bases-which greatly simplifies the customization of a DNA-binding domain recognizing a specific target sequence. In some embodiments, the TALEN may comprise a nuclease domain from a restriction endonuclease. For example, the restriction endonuclease is FokI. In some embodiments, the nuclease domain may dimerize to be active, and a pair of TALENS is designed for targeting a target sequence, which comprises two half target sequences recognized by each DNA binding domain on opposite strands of the DNA molecule, with an interconnecting sequence in between. For example, each half target sequence is in the range of 10 to 20 bp, and the interconnecting sequence is 12 to 19 bp in length. When both TALENs of the pair bind, the nuclease domain may dimerize and introduce a DSB within the interconnecting sequence. In some embodiments, the dimerization domain of the nuclease domain may comprise a knob-into-hole motif to promote dimerization. For example, the TALEN may comprise a knob-into-hole motif in the dimerization domain of FokI.

### 3. Target Sites

In some embodiments, the site-directed nucleases described herein are directed to and cleave (e.g., introduce a DSB) a target nucleic acid molecule (e.g. endogenous gene). In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid molecule is a blood-lineage gene. In some embodiments, the blood-lineage gene is protein tyrosine phosphatase receptor type C (PTPRC), IL2RG, or IL2RB. In some embodiments, the target nucleic acid is a gene associated with rapamycin response. In some embodiments, the target nucleic acid is FKBP12. In some embodiments, the target nucleic acid is B2M, TRAC or SIRPA.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

In some embodiments, the target sequence may be adjacent to a protospacer adjacent motif (PAM), a short sequence recognized by a CRISPR/Cas complex. In some embodiments, the PAM may be adjacent to or within 1, 2, 3, or 4, nucleotides of the 3′ end of the target sequence. In some embodiments, the target sequence may include the PAM. The length and the sequence of the PAM may depend on the Cas protein used. For example, the PAM may be selected from a consensus or a particular PAM sequence for a specific Cas nuclease or Cas ortholog, including those disclosed in FIG. 1 of Ran et al., (2015) Nature, 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (St1Cas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), and NNNNGATT (NmCas9) (see e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLOS ONE 7: e40913; Esvelt et al., (2013) Nat Methods 10 (11): 1116-1121 (wherein N is defined as any nucleotide, W is defined as either A or T, R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

In some embodiments, the PAM sequence that is recognized by a nuclease, e.g., Cas9, differs depending on the particular nuclease and the bacterial species it is from. In some embodiments, the PAM sequence recognized by SpCas9 is the nucleotide sequence 5′-NGG-3′, where “N” is any nucleotide. In some embodiments, a PAM sequence recognized by SaCas9 is the nucleotide sequence 5′-NGRRT-3′ or the nucleotide sequence 5′-NGRRN-3′, where “N” is any nucleotide and “R” is a purine (e.g., guanine or adenine). In some embodiments, a PAM sequence recognized by NmeCas9 is the nucleotide sequence 5′-NNNNGATT-3′, where “N” is any nucleotide. In some embodiments, a PAM sequence recognized by CjCas9 is the nucleotide sequence 5′-NNNNRYAC-3′, where “N” is any nucleotide, “R” is a purine (e.g., guanine or adenine), and “Y” is a pyrimidine (e.g., cytosine or thymine). In some embodiments, a PAM sequence recognized by StCas9 is the nucleotide sequence 5′-NNAGAAW-3′, where “N” is any nucleotide and “W” is adenine or thymine.

In some embodiments, the recombinant nuclease is Cas9 and the PAM sequence is the nucleotide sequence: (a) 5′-NGG-3′; (b) 5′-NGRRT-3′ or 5′-NGRRN-3′; (c) 5′-NNNNGATT-3′; (d) 5′-NNNNRYAC-3′; or (e) 5′-NNAGAAW-3′; where “N” is any nucleotide, “R” is a purine (e.g., guanine or adenine), “Y” is a pyrimidine (e.g., cytosine or thymine), and “W” is adenine or thymine. In some embodiments, the recombinant nuclease is Cas9, e.g., SpCas9, and the PAM sequence is 5′-NGG-3′, where “N” is any nucleotide. In some embodiments, the recombinant nuclease is Cas9, e.g., SaCas9, and the PAM sequence is 5′-NGRRT-3′ or 5′-NGRRN-3′, where “N” is any nucleotide and “R” is a purine, such as guanine or adenine. In some embodiments, the recombinant nuclease is Cas9, e.g., NmeCas9, and the PAM sequence is 5′-NNNNGATT-3′, where “N” is any nucleotide. In some embodiments, the recombinant nuclease is Cas9, e.g., CjCas9, and the PAM sequence is 5′-NNNNRYAC-3′, where “N” is any nucleotide, “R” is a purine, such as guanine or adenine, and “Y” is a pyrimidine, such as cytosine or thymine. In some embodiments, the recombinant nuclease is Cas9, e.g., StCas9, and the PAM sequence is 5′-NNAGAAW-3′, where “N” is any nucleotide and “W” is adenine or thymine.

### 4. Ribonucleoproteins

In some embodiments, the site-directed polypeptide (e.g., Cas nuclease) and genome-targeting nucleic acid (e.g., gRNA or sgRNA) may each be administered separately to a cell or a subject. In some embodiments, the site-directed polypeptide may be pre-complexed with one or more guide RNAs, or one or more sgRNAs. Such pre-complexed material is known as a ribonucleoprotein particle (RNP). In some embodiments, the nuclease system comprises a ribonucleoprotein (RNP). In some embodiments, the nuclease system comprises a Cas9 RNP comprising a purified Cas9 protein in complex with a gRNA. In some embodiments, the nuclease system comprises a Mad7 RNP comprising a purified Mad7 protein in complex with a gRNA. Cas9 and Mad7 protein can be expressed and purified by any means known in the art. Ribonucleoproteins are assembled in vitro and can be delivered directly to cells using standard electroporation or transfection techniques known in the art.

### B. Homology-Directed Repair (HDR)

In some aspects, the provided embodiments involve targeted integration of a nucleic acid sequence, such as a donor template, at a target nucleic acid sequence, e.g. an endogenous gene. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is B2M, TRAC or SIRPA. In some embodiments, the target nucleic acid is B2M.

In some embodiments, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break.

In some embodiments, HDR is utilized for targeted integration or insertion of a nucleic acid sequence(s), e.g., a donor template, in one or more target nucleic acid molecules (e.g., endogenous gene(s)). In some embodiments, HDR can be used to integrate a donor template comprising a synthetic cytokine receptor (e.g., a RACR) into a target nucleic acid molecule (e.g. an endogenous gene). For example, HDR can be used to integrate a donor template encoding a RACR into the B2M gene locus.

Agents capable of inducing a DSB, such as Cas nucleases (e.g. Cas9), TALENs, and ZFNs, promote genomic editing by inducing a DSB at a cleavage site within a target nucleic acid molecule such as an endogenous gene, e.g., B2M, as discussed in preceding sections.

Agents capable of inducing a SSB, also sometimes referred to as a nick, include recombinant nucleases, e.g., Cas9, having nickase activity, such as, e.g., those described in preceding sections. Examples of agents having nickase activity includes, e.g., a Cas9 from Streptococcus pyogenes that comprises a mutation selected from the group consisting of D10A, H840A, H854A, and H863A.

Upon cleavage by one of these agents, the target endogenous gene, e.g., B2M, with the SSBs or the DSB undergoes one of two major pathways for DNA damage repair: (1) the error-prone non-homologous end joining (NHEJ), or (2) the high-fidelity homology-directed repair (HDR) pathway.

In some embodiments, cells in which SSBs or a DSB was previously induced by one or more agent(s) comprising a nuclease, are obtained, and a donor template, e.g., ssODN, is introduced to result in HDR and integration of the donor template into the target endogenous gene, e.g., B2M.

In general, in the absence of a repair template, e.g., a donor template, such as a ssODN, the NHEJ process re-ligates the ends of the cleaved DNA strands, which frequently results in nucleotide deletions and insertions at the cleavage site.

Alteration of nucleic acid sequences at target endogenous gene locus, such as the B2M gene locus, can occur by HDR by integrating an exogenously provided donor template that encodes for a synthetic cytokine receptor (e.g., a RACR). The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., donor template, is typically double-stranded. In canonical HDR, a double-stranded polynucleotide, e.g., a double stranded donor template, is introduced, which comprises a sequence that is homologous to the targeting sequence within the target endogenous gene locus, and which will either be directly integrated into the targeting sequence or will be used as a template to insert the sequence, or a portion the sequence, of the donor template into the target endogenous gene, e.g., B2M After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotide, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., donor template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a donor template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., donor template, whereas canonical HDR generally involves a double-stranded homologous template. In the alternative HDR pathway, a single strand template polynucleotide, e.g., donor template, is introduced. A nick, single strand break, or DSB at the cleavage site, for altering a desired target site, e.g., a target endogenous gene, e.g., B2M, is mediated by a nuclease molecule, e.g., any of the nucleases as described herein, and resection at the break occurs to reveal single stranded overhangs. Incorporation of the sequence of the template polynucleotide, e.g., donor template, to alter the target site of the DNA typically occurs by the SDSA pathway, as described herein.

In some embodiments, HDR is carried out by introducing, into a cell, one or more agent(s) capable of inducing a DSB, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

In some embodiments, HDR is carried out by introducing, into a cell, one or more agent(s) capable of inducing a SSB in each stand, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

### Donor Templates

In some embodiments, the provided methods include the use of a donor template, e.g., a donor template encoding a synthetic cytokine receptor, e.g., a RACR, that is homologous to a portion(s) of the targeting sequence in the target gene, e.g., B2M. In some embodiments, the targeting sequence is comprised within the sense strand. In some embodiments, the targeting sequence is comprised within the antisense strand. Also provided, in some embodiments, are donor templates for use in the methods provided herein, e.g., as templates for HDR-mediated integration of a nucleic acid sequence encoding a RACR.

In some embodiments, the donor template is used in conjunction with the one or more agent(s) capable of inducing a DNA break, e.g., a SSB or a DSB. In some embodiments, the donor template is used in conjunction with the one or more agent(s) capable of inducing a DSB and a guide RNA, e.g., sgRNA, to knock in a nucleic acid sequence encoding a synthetic cytokine receptor (e.g., a RACR) at a target endogenous gene locus (e.g., B2M). In some embodiments, the donor template is used in conjunction with the one or more agent(s) capable of inducing an SSB; the first guide RNA, e.g., the first sgRNA; and the second guide RNA, e.g., the second sgRNA, to knock in a nucleic acid sequence encoding a synthetic cytokine receptor (e.g., a RACR) at a target endogenous gene locus (e.g., B2M).

In some embodiments, the donor template comprises a nucleic acid sequence that is homologous to the cleavage site in the target gene, e.g., B2M. In some embodiments, the donor template comprises a nucleic acid sequence that is homologous to the cleavage site in the sense strand of the target gene, e.g., B2M. In some embodiments, the donor template comprises a nucleic acid sequence that is homologous to the cleavage site in the antisense strand of the target gene, e.g., B2M. In some embodiments, the target gene, e.g., B2M, includes a sense strand and an antisense strand, and the sense strand comprises the targeting sequence. In some embodiments, the target gene, e.g., B2M, includes a sense strand and an antisense strand, and the antisense strand comprises the targeting sequence.

In some embodiments, the donor template, e.g., ssODN, comprises a nucleic acid sequence comprising a PAM sequence that is homologous to the PAM sequence in the targeting sequence.

In some embodiments, the donor template is single-stranded. In some embodiments, the donor template is a single-stranded DNA oligonucleotide (ssODN). In some embodiments, the donor template is double-stranded.

In some embodiments, the ssODN comprises a 5′ ssODN arm and a 3′ ssODN arm. In some embodiments, the 5′ ssODN arm is directly linked to the 3′ ssODN arm. In some embodiments, the 5′ ssODN arm is homologous to the sequence of the target gene, e.g., B2M, that is immediately upstream of the cleavage site, and the 3′ ssODN arm is homologous to the sequence of the target gene that is immediately downstream of the cleavage site.

In some embodiments, the 5′ ssODN arm and/or the 3′ ssODN arm has a length that is between 250 and 750 nucleotides in length. In some embodiments, the 5′ ssODN arm has a length that is between 250 and 750 nucleotides in length. In some embodiments, the 3′ ssODN arm has a length that is between 250 and 750 nucleotides in length. In some embodiments, each of the 5′ ssODN arm and the 3′ ssODN arm has a length that is between 250 and 750 nucleotides in length. In some embodiments, the 5′ ssODN arm and/or the 3′ ssODN arm has a length that is about 500 nucleotides in length. In some embodiments, the 5′ ssODN arm has a length that is about 500 nucleotides in length. In some embodiments, the 3′ ssODN arm has a length that is about 500 nucleotides in length. In some embodiments, each of the 5′ ssODN arm and the 3′ ssODN arm has a length that is about 500 nucleotides in length.

In some embodiments, the target gene is B2M and the donor template comprises a nucleic acid sequence that is homologous to the cleavage site in the B2M gene. In some embodiments, the donor template comprises a nucleic acid sequence that is homologous to the cleavage site in the sense strand of the B2M target gene. In some embodiments, the donor template comprises a nucleic acid sequence that is homologous to the cleavage site in the antisense strand of the B2M target gene. In some embodiments, the donor template is a ssODN and the 5′ ssODN arm is homologous to the sequence of the B2M target gene that is immediately upstream of the cleavage site, and the 3′ ssODN arm is homologous to the sequence of the B2M target gene that is immediately downstream of the cleavage site.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 22. In some embodiments, the 5′ ssODN arm comprises the nucleic acid sequence set forth in SEQ ID NO:22.

In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 23 In some embodiments, the 3′ ssODN arm comprises the nucleic acid sequence set forth in SEQ ID NO:23.

In some embodiments, the 5′ ssODN arm comprises the nucleic acid sequence as set forth in SEQ ID NO: 22, and the 3′ ssODN arm comprises the nucleic acid sequence as set forth in SEQ ID NO: 23.

Also provided herein is an isolated nucleic acid, e.g., an isolated nucleic acid for use in a method of knocking in a synthetic cytokine receptor (e.g., a RACR) into a target gene (e.g., B2M), comprising the nucleic acid sequence of any of the donor templates, e.g., ssODNs, or portions thereof, e.g., or 5′ ssODN arms, or 3′ ssODN arms, described herein. In some embodiments, the 5′ ssODN comprises the nucleic acid sequence as set forth in SEQ ID NO:22. In some embodiments, the 3′ ssODN arm comprises the nucleic acid sequence as set forth in SEQ ID NO: 23.

In some embodiments, the crRNA comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 18; the 5′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the 3′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23. In some embodiments, the crRNA comprises the nucleic acid sequence set forth in any one of SEQ ID NOS: 18; the 5′ ssODN comprises the nucleic acid sequence set forth in SEQ ID NO: 22; and the 3′ ssODN comprises the nucleic acid sequence set forth in SEQ ID NO: 23.

In some embodiments, the donor template, e.g., ssODN, comprises a nucleic acid sequence encoding a transgene sequence encoding the synthetic cytokine receptor. In some embodiments, the synthetic cytokine receptor is a rapamycin-activated cytokine receptor (RACR) that is responsive to rapamycin or an analog (e.g. rapalog). In some embodiments, the transgene sequence is a tandem cassette that encodes both polypeptides of the synthetic cytokine receptor.

In some embodiments, the transgene encoding the synthetic cytokine receptor (e.g. RACR) can be inserted so that its expression is driven by the endogenous promoter at the integration site, for example the promoter that drives expression of the endogenous B2M gene. In some embodiments in which the polypeptide encoding sequences are promoterless, expression of the integrated transgene is then ensured by transcription driven by an endogenous promoter or other control element in the region of interest. For example, the transgene encoding a portion of the synthetic cytokine receptor (e.g. RACR) can be inserted without a promoter, but in-frame with the coding sequence of the endogenous locus (e.g. B2M locus) such that expression of the integrated transgene is controlled by the transcription of the endogenous promoter and/or other regulatory elements at the integration site. In some embodiments, a multicistronic element such as a ribosome skipping element/self-cleavage element (e.g., a 2A element or an internal ribosome entry site (IRES)), is placed upstream of the transgene, such that the multicistronic element is placed in-frame with one or more exons of the endogenous open reading frame at the endogenous locus (e.g. B2M locus), such that the expression of the transgene is operably linked to the endogenous promoter.

In some embodiments, each nucleic acid encoding a polypeptide of the synthetic cytokine receptor in the “tandem” cassettes is independently controlled by a regulatory element or all controlled as a multi-cistronic (e.g. bicistronic) expression system. In other embodiments, each nucleic acid encoding a polypeptide of the synthetic cytokine receptor in the “tandem” cassettes can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two polypeptides separated from one another by sequences encoding a cleavable linker as described herein. The ORF thus encodes a single polypeptide, which, either during or after translation, is processed into the individual polypeptide chains. In some embodiments, the promoter is selected from among human elongation factor 1 alpha (EF1α) promoter (such as set forth in SEQ ID NO:24, 25 or 26). In some embodiments, the promoter is an MND promoter (such as set forth in SEQ ID NO: 27).

In some embodiments, the donor template, e.g., ssODN, comprises a nucleic acid sequence encoding a synthetic cytokine receptor (e.g. RACR). In some embodiments, the nucleic acid sequence encoding the synthetic cytokine receptor (e.g. RACR) is located between the 5′ ssODN arm and the 3′ ssODN arm. In some embodiments, the nucleic acid sequence encoding the synthetic cytokine receptor (e.g. RACR) comprises an EF1-alpha promoter (e.g., SEQ ID NO:24, 25 or 26). In some embodiments, the nucleic acid sequence encoding the synthetic cytokine receptor (e.g. RACR) comprises a MND promoter (e.g., SEQ ID NO:27). In some embodiments, the synthetic cytokine receptor is a rapamycin-activated cytokine receptor (RACR). The RACR can be any as described, such as in Section V. In some embodiments, the nucleic acid molecule is a tandem cassette encoding the first polypeptide sequence of RACR and the second polypeptide sequence of RACR.

In some embodiments, the first nucleic acid sequence encoding the RACR comprises a nucleic acid sequence encoding a RACR-gamma chain (e.g., SEQ ID NO: 28), and a nucleic acid sequence encoding a RACR-beta chain (e.g., SEQ ID NO: 33). In some embodiments, the first nucleic acid sequence encodes a RACR-gamma chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 28. In some embodiments, the first nucleic acid sequence encodes the RACR-gamma chain sequence set forth in SEQ ID NO:28. In some embodiments, the nucleic acid sequence encoding the RACR-gamma chain further encodes a signal peptide at the N-terminus of the nascent protein to prompt transport of the protein when expressed. In some embodiments, the signal peptide has the sequence set forth in SEQ ID NO: 29. In some embodiments, the second nucleic acid sequence encodes a RACR-beta chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In some embodiments, the second nucleic acid sequence encodes the RACR-beta chain set forth in SEQ ID NO: 33. In some embodiments, the nucleic acid sequence encoding the RACR-beta chain further encodes a signal peptide at the N-terminus of the nascent protein to prompt transport of the protein when expressed. In some embodiments, the signal peptide has the sequence set forth in SEQ ID NO: 34.

In some embodiments, the first nucleic acid sequence encoding the RACR-gamma chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37. In some embodiments, the first nucleic acid sequence encoding the RACR-gamma chain has the sequence set forth in SEQ ID NO:37. In some embodiments, the second nucleic acid sequence encoding the RACR-beta chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38. In some embodiments, the second nucleic acid sequence encoding the RACR-beta chain is set forth in SEQ ID NO:38.

In some embodiments, the nucleic acid sequence encoding the RACR-gamma chain and the nucleic acid sequence encoding the RACR-beta chain are separated by a nucleic acid sequence encoding a cleavable linker. In some embodiments, a further nucleic acid sequence encoding a cleavable linker is located downstream of the nucleic acid sequence encoding the RACR-beta chain

In some embodiments, the linker is a protein quantitation reporter linker (PQR; e.g., SEQ ID NO:42), including any as described in Canadian Patent Application No. CA2970093, incorporated by reference in its entirety herein. In some embodiments, the PQR linker has the sequence set forth in SEQ ID NO:42. In some embodiments, the PQR linker is encoded by a sequence of nucleotides set forth in SEQ ID NO:41.

In some embodiments, the cleavable linker is a self-cleaving peptide, such as a 2A ribosomal skip element. In some cases, the cleavable linker, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 43), equine rhinitis A virus (E2A, e.g., SEQ ID NO: 44), Thosea asigna virus (T2A, e.g., SEQ ID NO: 45 or 46), and porcine teschovirus-1 (P2A, e.g., SEQ ID NO: 47 or 48) as described in U.S. Patent Publication No. 20070116690.

In some embodiments, by virtue of the cleavable element located between the first nucleic acid sequence and the second nucleic acid sequence, expression of a nucleic acid sequence encoding a RACR yields a first peptide (i.e., the RACR-gamma chain) and a separate, second peptide (i.e., the RACR-beta chain).

In some embodiments, the transgene sequences may also include sequences required for transcription termination and/or polyadenylation signal. In some aspects, exemplary polyadenylation signal is selected from SV40, hGH, BGH, and rbGlob transcription termination sequence and/or polyadenylation signal. In some embodiments, the transgene includes an SV40 polyadenylation signal. In some embodiments, if present within the transgene, the transcription termination sequence and/or polyadenylation signal is typically the most 3′ sequence within the transgene and is linked to one of the homology arm. In some embodiments, transgene sequence includes the polyadenylation sequence set forth in SEQ ID NO:39.

In some embodiments, the ssODN comprises, in order: a 5′ ssODN arm, a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, a polyA sequence, and the 3′ ssODN arm.

In some embodiments, the ssODN comprises the sequence set forth in SEQ ID NO: 40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40. In some embodiments, the ssODN is set forth in SEQ ID NO:40.

In some embodiments, after the integration of the ssODN into the target gene, the target gene is knocked out. In some embodiments, the target gene is human B2M, and, after the integration of the ssODN into B2M, B2M is knocked out. In some embodiments, a nucleic acid sequence encoding the synthetic cytokine receptor is integrated into the B2M locus. In some embodiments, the engineered iPSC and CIL has a modified B2M locus in which the endogenous B2M gene is genetically disrupted by knockout of the B2M gene and knock in by targeted integration of a nucleic acid encoding the synthetic cytokine receptor. In some embodiments, the synthetic cytokine receptor is a RACR encoded by a nucleic acid sequence that contains in order: a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, and a polyA sequence. In some embodiments, the nucleic acid sequence encoding RACR that is integrated into the B2M locus has the sequence set forth in SEQ ID NO:32 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 32. In some embodiments, the nucleic acid encoding RACR that is integrated into the B2M locus is set forth in SEQ ID NO:32.

### VII. Chimeric Antigen Receptor

In some cases, the engineered lymphocytes, such as CIL cells, of the present disclosure may comprise or be transduced with a polynucleotide encoding a chimeric antigen receptor (CAR), thereby generating lymphocytes, such as CIL cells, expressing the CAR.

In some embodiments, the disclosure contemplates a chimeric antigen receptor (CAR) system for use in the treatment of subjects with cancer. In some embodiments, the engineered lymphocytes, such as CIL cells, of the disclosure comprise a CAR sequence (CAR-CIL cells or CAR-iCIL cells).

In some embodiments, the lymphocytes, such as CIL cells, are engineered to express CAR constructs by transfecting a population of cells with an expression vector encoding the CAR construct. Illustrative examples of populations of cells that may be transfected include HSCs, blood progenitor cells, common lymphoid progenitor cells, or CIL cells. Appropriate means for preparing a transduced population of lymphocytes, such as CIL cells, expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples. In some embodiments, any of the transduction methods contemplated in the disclosure may be used to generate CAR-expressing CIL cells.

### A. Targeting Agents for CARS

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFv's for innumerable haptens are known in the art or can be generated by conventional methods routinely. Accordingly, the present disclosure contemplates using any known hapten-binding domain.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR-CIL cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In some embodiments, the CAR system of the disclosure makes use of CARs that target a moiety that is not produced or expressed by cells of the subject being treated. This CAR system thus allows for focused targeting of the CIL cells to target cells, such as cancer cells. By administration of a small conjugate molecule along with the CAR-expressing cells, such as CAR-expressing CIL cells, the cell response of the engineered cells can be targeted to only those cells expressing the tumor receptor, thereby reducing off-target toxicity, and the activation of the engineered lymphocytes, e.g. CIL cells, can be more easily controlled due to the rapid clearance of the small conjugate molecule. As an added advantage, the CAR-expressing lymphocytes, such as CAR-expressing CIL cells, can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

In one embodiment, the disclosure provides an illustration of this conjugate molecule/CAR system.

In some embodiments, the CAR system of the disclosure utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275 (11): 26684-90), centyrins, and DARPins (see Reichert, J. M. MAbs 2009. 1 (3): 190-209).

In some embodiments, the cell-targeting moiety is DUPA (DUPA-(99m) Tc), a ligand bound by PSMA-positive human prostate cancer cells with nanomolar affinity (K_{D}=14 nM; see Kularatne, S. A. et al., Mol Pharm. 2009. 6 (3): 780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega. E. I. et al., Cancer Metastasis Rev. 2008. 27 (4): 655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according to the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

An illustrative conjugate of the disclosure is FITC-Folate

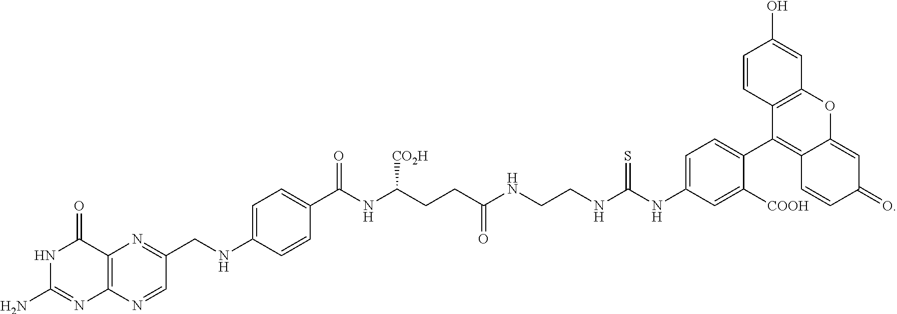 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

An illustrative conjugate of the disclosure is FITC-CA9

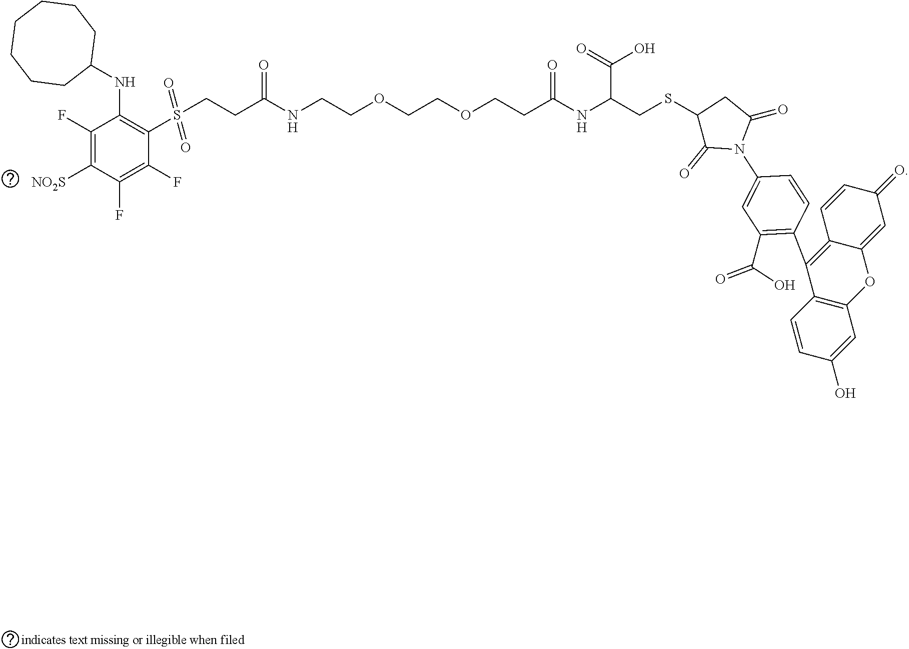

Illustrative conjugates of the disclosure include the following molecules: FITC-(PEG) 12-Folate, FITC-(PEG) 20-Folate, FITC-(PEG) 108-Folate, FITC-DUPA, FITC-(PEG) 12-DUPA, FITC-CCK2R ligand, FITC-(PEG) 12-CCK2R ligand, FITC-(PEG) 11-NK1R ligand and FITC-(PEG) 2-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 pM or 10 pM, even more preferably at least about 100 pM.

Examples of conjugates and methods of making them are provided in U.S. patent applications US 2017/0290900, US 2019/0091308, and US 2020/0023009, all of which are incorporated herein by reference.

### B. CAR Constructs

In some embodiments, the CAR construct contains an extracellular binding portion, a transmembrane domain and an intracellular signaling domain. In some embodiments, the intracellular signaling domain contains a costimulatory signaling domain and/or an activation signaling domain. In some embodiments, the CAR construct contains an extracellular binding portion, a transmembrane domain and an intracellular signaling domain comprising a costimulatory signaling domain. In some embodiments, the CAR construct contains an extracellular binding portion, a transmembrane domain and an intracellular signaling domain comprising an activation signaling domain. In some embodiments, the CAR construct contains an extracellular binding portion, a transmembrane domain and an intracellular signaling domain comprising a costimulatory signaling domain and an activation signaling domain.

In any embodiments described herein, the binding portion of the CAR can be, for example, a single chain fragment variable region (scFv) of an antibody, a Fab, Fv. Fc, or (Fab′) 2 fragment, and the like. The use of unaltered (i.e., full size) antibodies, such as IgG, IgM, IgA, IgD or IgE, in the CAR or as the CAR is excluded from the scope of the invention.

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina. L. et al., Eur J Imnumiol. 1996. 26 (10): 2304-9); CD137 (4-1BB), a member of the tumor necrosis factor (TNF) receptor family (see, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments of the invention, the CAR constructs comprise two co-stimulation domains. While the particular combinations include all possible variations of the four noted domains, specific examples include: 1) CD28+CD137 (4-1BB) and 2) CD28+ CD134 (OX40).

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. Suitable activation signaling domains include the CD35 chain and Fc receptor γ. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

Illustrative CAR constructs suitable for CAR-engineered lymphocytes, such as CAR-CIL cells, are provided below:
(1) scFv-CD8_{TM}-4-1BB_{IC}-CD3ζs (see, e.g., Liu E, Tong Y, Dotti G, et al., Leukemia. 2018; 32:520-531);
(2) scFv-CD28_{TM+IC}-CD3ζs (see, e.g., Han J, Chu J, Keung C W et al., Sci Rep. 2015; 5:11483; Kruschinski A, Moosmann A, Poschke I et al., Proc Natl Acad Sci USA. 2008; 105:17481-17486; and Chu J, Deng Y, Benson D M et al., Leukemia. 2014; 28:917-927);
(3) scFv-DAP12_{TM+IC} (see, e.g., Muller N, Michen S, Tietze S et al., J Immunother. 2015; 38:197-210);
(4) scFv-CD8_{TM}-2B4_{IC}-CD3ζs (see, e.g., Xu Y, Liu Q, Zhong M et al., J Hematol Oncol. 2019; 12:49);
(5) scFv-2B4_{TM+IC}-CD3ζs (see, e.g., Altvater B, Landmeier S, Pscherer S et al., Clin Cancer Res. 2009; 15:4857-4866);
(6) scFv-CD28_{TM+IC}-4-1BB_{IC}-CD3ζs (see, e.g., Kloss S, Oberschmidt O, Morgan M et al., Hum Gene Ther. 2017; 28:897-913);
(7) scFv-CD16_{TM}-2B4_{IC}-CD3ζs (see, e.g., Li Y, Hermanson D L, Moriarity B S Kaufman D S, Cell Stem Cell. 2018; 23:181-192);
(8) scFv-NKp44_{TM}-DAP10_{IC}-CD3ζs (see, e.g., Li Y, Hermanson D L, Moriarity B S Kaufman D S, Cell Stem Cell. 2018; 23:181-192);
(9) scFv-NKp46_{TM}-2B4_{IC}-CD3ζs (see, e.g., Li Y, Hermanson D L, Moriarity B S Kaufman D S, Cell Stem Cell. 2018; 23:181-192);
(10) scFv-NKG2D_{TM}-2B4_{IC}-CD3ζs (see, e.g., Li Y, Hermanson D L, Moriarity B S Kaufman D S, Cell Stem Cell. 2018; 23:181-192);
(11) scFv-NKG2D_{TM}-4-1BB_{IC}-CD3ζs (see, e.g., Li Y, Hermanson D L, Moriarity B S Kaufman D S, Cell Stem Cell. 2018; 23:181-192);
(12) scFv-NKG2D_{TM}-2B4IC-DAP12_{IC}-CD3ζs (see, e.g., Li Y, Hermanson D L, Moriarity B S Kaufman D S, Cell Stem Cell. 2018; 23:181-192);
(13) scFv-NKG2D_{TM}-2B4IC-DAP10_{IC}-CD3ζs (see, e.g., Li Y, Hermanson D L, Moriarity B S Kaufman D S, Cell Stem Cell. 2018; 23:181-192);
(14) scFv-NKG2D_{TM}-4-1BB_{IC}-2B4_{IC}-CD3ζS (see, e.g., Li Y, Hermanson D L, Moriarity B S Kaufman D S, Cell Stem Cell. 2018; 23:181-192); and
(15) scFv-NKG2D_{TM}-CD3ζS (see, e.g., Li Y, Hermanson D L, Moriarity B S Kaufman D S, Cell Stem Cell. 2018; 23:181-192).

In some embodiments, the binding portion of the CAR can be directed to any antigen that is desired to be targeted, such as due to its overexpression on cells or association with a disease or conditions like cancer.

In some embodiments, the binding portion of the CAR is specific to a tumor antigen. The selection of the antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewis Y, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1. XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, IL13Ra2, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

A skilled artisan is readily familiar with CARs against diverse tumor antigens. Any one of such CARs can be employed as the CAR. Numerous CARs have been approved by the FDA and include, but are not limited to, anti-CD19 and anti-BCMA CAR T cells such as tisagenlecleucel (Kymriah), axicabtagene ciloleucel (Yescarta), brexucabtagene autoleucel (Tecartus), lisocabtagene maraleucel (Breyanzi), or idecabtagene vicleucel (Abecma). It is within the level of a skilled artisan to generate similar constructs for specific targeting of a desired tumor antigen.

In some embodiments, the binding portion of the CAR can be directed to a universal antigen to target a wide variety of tumors without the need to prepare separate CAR constructs. The targeted moiety recognized by the CAR may also remain constant. In some embodiments, a ligand may be administered to the subject to allow interaction with target cells and interaction with the binding portion of the CAR. It is only the ligand portion of the small conjugate molecule that needs to be altered to allow the system to target cancer cells of different identity. Exemplary universal CAR systems are described in the section above.

In some embodiments, the CAR is an anti-FITC CAR and the ligand is composed of a fluorescein or fluorescein isothiocyanate (FITC) moiety conjugated to an agent that binds to a desired target cell (such as a cancer cell). Exemplary ligands are described in the section above. In some embodiments, the ligand is FITC-folate.

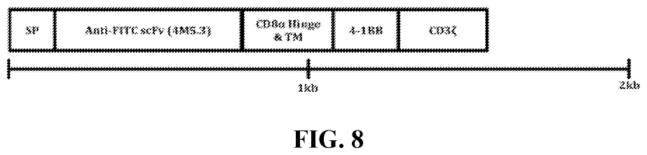

An illustrative CAR of the disclosure is shown in FIG. 8 where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD3ζ.

An illustrative nucleotide sequence encoding a CAR may comprise SEQ ID
NO: 13:
(SEQ ID NO: 13)
ATGGCCTTACCAGTGACCGCCTTGCTCCTGCCGCTGGCCTTGCTGCTCCAC
GCCGCCAGGCCGGATGTCGTGATGACCCAGACCCCCCTCAGCCTCCCAGT
GTCCCTCGGTGACCAGGCTTCTATTAGTTGCAGATCCAGCCAGTCCCTCGT
GCACTCTAACGGTAATACCTACCTGAGATGGTATCTCCAGAAGCCCGGAC
AGAGCCCTAAGGTGCTGATCTACAAAGTCTCCAACCGGGTGTCTGGAGTC
CCTGACCGCTTCTCAGGGAGCGGTTCCGGCACCGACTTCACCCTGAAGAT
CAACCGGGTGGAGGCCGAAGACCTCGGCGTCTATTTCTGCTCTCAGAGTA
CACATGTGCCCTGGACCTTCGGCGGAGGGACCAAGCTGGAGATCAAAAGC
TCCGCAGACGATGCCAAGAAAGATGCCGCTAAGAAAGACGATGCTAAGA
AAGACGATGCAAAGAAAGACGGTGGCGTGAAGCTGGATGAAACCGGAGG
AGGTCTCGTCCAGCCAGGAGGAGCCATGAAGCTGAGTTGCGTGACCAGCG
GATTCACCTTTGGGCACTACTGGATGAACTGGGTGCGACAGTCCCCAGAG
AAGGGGCTCGAATGGGTCGCTCAGTTCAGGAACAAACCCTACAATTATGA
GACATACTATTCAGACAGCGTGAAGGGCAGGTTTACTATCAGTAGAGACG
ATTCCAAATCTAGCGTGTACCTGCAGATGAACAATCTCAGGGTCGAAGAT
ACAGGCATCTACTATTGCACAGGGGCATCCTATGGTATGGAGTATCTCGG
TCAGGGGACAAGCGTCACAGTCAGTTTCGTGCCGGTCTTCCTGCCAGCGA
AGCCCACCACGACGCCAGCGCCGCGACCACCAACACCGGCGCCCACCATC
GCGTCGCAGCCCCTGTCCCTGCGCCCAGAGGCGTGCCGGCCAGCGGCGGG
GGGCGCAGTGCACACGAGGGGGCTGGACTTCGCCTGTGATATCTACATCT
GGGCGCCCTTGGCCGGGACTTGTGGGGTCCTTCTCCTGTCACTGGTTATCA
CCCTTTACTGCAACCACAGGAACCGTTTCTCTGTTGTTAAACGGGGCAGA
AAGAAACTCCTGTATATATTCAAACAACCATTTATGAGACCAGTACAAAC
TACTCAAGAGGAAGATGGCTGTAGCTGCCGATTTCCAGAAGAAGAAGAA
GGAGGATGTGAACTGAGAGTGAAGTTCAGCAGGAGCGCAGACGCCCCCG
CGTACCAGCAGGGCCAGAACCAGCTCTATAACGAGCTCAATCTAGGACGA
AGAGAGGAGTACGATGTTTTGGACAAGAGACGTGGCCGGGACCCTGAGA
TGGGGGGAAAGCCGAGAAGGAAGAACCCTCAGGAAGGCCTGTACAATGA
ACTGCAGAAAGATAAGATGGCGGAGGCCTACAGTGAGATTGGGATGAAA
GGCGAGCGCCGGAGGGGCAAGGGGCACGATGGCCTTTACCAGGGTCTCA
GTACAGCCACCAAGGACACCTACGACGCCCTTCACATGCAGGCCCTGCCC
CCTCGCTAA
An illustrative CAR amino acid sequence may comprise SEQ ID NO: 14:
(SEQ ID NO: 14)
MALPVTALLLPLALLLHAARPDVVMTQTPLSLPVSLGDQASISCRSSQSLVHS
NGNTYLRWYLQKPGQSPKVLIYKVSNRVSGVPDRFSGSGSGTDFTLKINRVE
AEDLGVYFCSQSTHVPWTFGGGTKLEIKSSADDAKKDAAKKDDAKKDDAK
KDGGVKLDETGGGLVQPGGAMKLSCVTSGFTFGHYWMNWVRQSPEKGLE
WVAQFRNKPYNYETYYSDSVKGRFTISRDDSKSSVYLQMNNLRVEDTGIYY
CTGASYGMEYLGQGTSVTVSFVPVFLPAKPTTTPAPRPPTPAPTIASQPLSLRP
EACRPAAGGAVHTRGLDFACDIYIWAPLAGTCGVLLLSLVITLYCNHRNRFS
VVKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
An illustrative nucleotide insert may comprise SEQ ID NO: 15:
(SEQ ID NO: 15)
GCCACCATGGCCTTACCAGTGACCGCCTTGCTCCTGCCGCTGGCCTTGCTG
CTCCACGCCGCCAGGCCGGATGTCGTGATGACCCAGACCCCCCTCAGCCT
CCCAGTGTCCCTCGGTGACCAGGCTTCTATTAGTTGCAGATCCAGCCAGTC
CCTCGTGCACTCTAACGGTAATACCTACCTGAGATGGTATCTCCAGAAGC
CCGGACAGAGCCCTAAGGTGCTGATCTACAAAGTCTCCAACCGGGTGTCT
GGAGTCCCTGACCGCTTCTCAGGGAGCGGTTCCGGCACCGACTTCACCCT
GAAGATCAACCGGGTGGAGGCCGAAGACCTCGGCGTCTATTTCTGCTCTC
AGAGTACACATGTGCCCTGGACCTTCGGCGGAGGGACCAAGCTGGAGATC
AAAAGCTCCGCAGACGATGCCAAGAAAGATGCCGCTAAGAAAGACGATG
CTAAGAAAGACGATGCAAAGAAAGACGGTGGCGTGAAGCTGGATGAAAC
CGGAGGAGGTCTCGTCCAGCCAGGAGGAGCCATGAAGCTGAGTTGCGTG
ACCAGCGGATTCACCTTTGGGCACTACTGGATGAACTGGGTGCGACAGTC
CCCAGAGAAGGGGCTCGAATGGGTCGCTCAGTTCAGGAACAAACCCTACA
ATTATGAGACATACTATTCAGACAGCGTGAAGGGCAGGTTTACTATCAGT
AGAGACGATTCCAAATCTAGCGTGTACCTGCAGATGAACAATCTCAGGGT
CGAAGATACAGGCATCTACTATTGCACAGGGGCATCCTATGGTATGGAGT
ATCTCGGTCAGGGGACAAGCGTCACAGTCAGTTTCGTGCCGGTCTTCCTGC
CAGCGAAGCCCACCACGACGCCAGCGCCGCGACCACCAACACCGGCGCC
CACCATCGCGTCGCAGCCCCTGTCCCTGCGCCCAGAGGCGTGCCGGCCAG
CGGCGGGGGGCGCAGTGCACACGAGGGGGCTGGACTTCGCCTGTGATATC
TACATCTGGGCGCCCTTGGCCGGGACTTGTGGGGTCCTTCTCCTGTCACTG
GTTATCACCCTTTACTGCAACCACAGGAACCGTTTCTCTGTTGTTAAACGG
GGCAGAAAGAAACTCCTGTATATATTCAAACAACCATTTATGAGACCAGT
ACAAACTACTCAAGAGGAAGATGGCTGTAGCTGCCGATTTCCAGAAGAAG
AAGAAGGAGGATGTGAACTGAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCC
TGAGATGGGGGGAAAGCCGAGAAGGAAGAACCCTCAGGAAGGCCTGTAC
AATGAACTGCAGAAAGATAAGATGGCGGAGGCCTACAGTGAGATTGGGA
TGAAAGGCGAGCGCCGGAGGGGCAAGGGGCACGATGGCCTTTACCAGGG
TCTCAGTACAGCCACCAAGGACACCTACGACGCCCTTCACATGCAGGCCC
TGCCCCCTCGCTAA

In some embodiments, the CAR may be encoded by a nucleic acid sequence that encodes a signal peptide to signal transport of the CAR in the cell. It is understood that typically the signal peptide is removed from the protein.

An illustrative CAR amino acid sequence without a signal peptide may comprise SEQ ID NO: 16:

(SEQ ID NO: 16)
DVVMTQTPLSLPVSLGDQASISCRSSQSLVHSNGNTYLRWYLQKPGQSPK
VLIYKVSNRVSGVPDRFSGSGSGTDFTLKINRVEAEDLGVYFCSQSTHVP
WTFGGGTKLEIKSSADDAKKDAAKKDDAKKDDAKKDGGVKLDETGGGLVQ
PGGAMKLSCVTSGFTFGHYWMNWVRQSPEKGLEWVAQFRNKPYNYETYYS
DSVKGRFTISRDDSKSSVYLQMNNLRVEDTGIYYCTGASYGMEYLGQGTS
VTVSFVPVFLPAKPTTTPAPRPPTPAPTIASQPLSLRPEACRPAAGGAVH
TRGLDFACDIYIWAPLAGTCGVLLLSLVITLYCNHRNRFSVVKRGRKKLL
YIFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPR

An illustrative CAR amino acid sequence signal peptide may comprise SEQ ID NO: 17:

(SEQ ID NO: 17)
MALPVTALLLPLALLLHAARP

In various embodiments, CAR-expressing cells comprising the nucleic acid of SEQ ID NO: 13 or 15 are provided. In some embodiments, a chimeric antigen receptor polypeptide comprising SEQ ID NO: 14 is contemplated. In some embodiments, a chimeric antigen receptor polypeptide comprising SEQ ID NO: 16 is contemplated. In some embodiments, a vector is contemplated comprising SEQ ID NO: 13 or 15. In some embodiments, a lentiviral vector is contemplated comprising SEQ ID NO: 13 or 15. In some embodiments, SEQ ID NO: 14 can comprise or consist of human or humanized amino acid sequences. In some embodiments, SEQ ID NO: 16 can comprise or consist of human or humanized amino acid sequences.

In some embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 13, SEQ ID NO: 14, SEQ ID NO: 15 or SEQ ID NO: 16 are contemplated.

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 pM, or 10 pM, preferably at least about 100 pM, 1 fM or 10 fM, even more preferably at least about 100 fM.

VII. Characteristics of iCIL Cells Expressing Synthetic Cytokine Receptors

In some embodiments, the CIL cells are CD3− CD5−, CD16+, CD56+, CD57+, NKp30+, NKp46+, NKG2A+, and/or NKG2D+.

In some embodiments, the population of engineered cells is 40% to 60% CD16+, 50% to 70% CD16+, 60% to 80% CD16+, 70% to 90% CD16+, 80% to 100% CD16+, or any percentage within a range defined by any two aforementioned values.

In some embodiments, the population of engineered cells is 60% to 80% CD56+, 65% to 85% CD56+, 70% to 90% CD56+, 75% to 95% CD56+, 80% to 99% CD56+, or any percentage within a range defined by any two aforementioned values.

In some embodiments, the population of engineered CIL cells is CD56lo. In some embodiments, the population of engineered CIL cells is CD56high.

In some embodiments, the population of engineered CIL cells is 60% to 80% CD16+ CD56+, 65% to 85% CD16+ CD56+, 70% to 90% CD16+ CD56+, 75% to 95% CD16+ CD56+, 80% to 99% CD16+ CD56+, or any percentage within a range defined by any two aforementioned values.

In some embodiments, the population of engineered CIL cells is at least 40% CD16+, at least 50% CD16+, at least 60% CD16+, at least 70% CD16+, at least 80% CD16+, at least 90% CD16+, or 100% CD16+.

In some embodiments, the population of engineered CIL cells is at least 80% CD56+, at least 85% CD56+, at least 90% CD56+, at least 95% CD56+, or 100% CD56+.

In some embodiments, the population of engineered CIL cells is at least 40% CD16+ CD56+, at least 50% CD16+ CD56+, at least 60% CD16+ CD56+, at least 70% CD16+ CD56+, at least 80% CD16+ CD56+, at least 90% CD16+ CD56+, or 100% CD16+ CD56+.

In some embodiments, CIL cells are characterized by being CD45+ CD56+.

In some embodiments, the population of engineered CIL cells is 40% to 60% CD45+, 50% to 70% CD45+, 60% to 80% CD45+, 70% to 90% CD45+, 80% to 100% CD45+, or any percentage within a range defined by any two aforementioned values.

In some embodiments, the population of engineered CIL cells is 60% to 80% CD45+ CD56+. 65% to 85% CD45+ CD56+, 70% to 90% CD45+ CD56+. 75% to 95% CD45+ CD56+, 80% to 99% CD45+ CD56+, or any percentage within a range defined by any two aforementioned values.

In some embodiments, the population of engineered CIL cells is at least 40% CD45+, at least 50% CD45+, at least 60% CD45+, at least 70% CD45+, at least 80% CD45+, at least 90% CD45+, or 100% CD45+.

In some embodiments, the population of engineered CIL cells is at least 40% CD45+ CD56+, at least 50% CD45+ CD56+, at least 60% CD45+ CD56+, at least 70% CD45+ CD56+, at least 80% CD45+ CD56+, at least 90% CD45+ CD56+, or 100% CD45+ CD56+.

K562 cells are a highly sensitive target for an in vitro cytotoxic innate lymphoid cell cytotoxic activity assay. In the assay, CIL cells are co-incubated at different ratios with K562 target tumor cells known to be sensitive to CIL cell-mediated cytotoxicity. The target cells (K562) are pre-labeled with a fluorescent dye to allow their discrimination from the effector cells (CIL cells). After the incubation period, killed target cells are identified by a nucleic acid stain, which specifically permeates dead cells. % Dead is calculated by comparing the total number of viable cells in each experimental assay well to a non-effector control well. As used herein, the term “activity” refers to a measurement of the CIL cells' cytotoxic capacity against target cells.

In some embodiments, the engineered CIL cells secrete CD107a in response to an antigen recognized by the engineered CIL cells.

Innate CIL cell receptor stimulation causes CIL cell secretion of CD107a. CD107a expression correlates with both cytokine secretion and CIL cell-mediated lysis of target cells and thus, as used herein, CD107a secretion is a marker of CIL cell functional activity.

In some embodiments, the engineered CIL cells secrete interferon gamma (IFNγ) and/or tumor necrosis factor alpha (TNF-α) in response to an antigen recognized by the engineered CIL cells.

CIL cell activation leads to the secretion of IFNγ and TNF-α which synergistically enhance CIL cell cytotoxicity. In engineered CIL cells, expression of IFNγ and/or TNF-α are markers of CIL cell activity.

As described above, provided herein are hematopoietic stem or progenitor cells that may be differentiated into lymphoid cells using a synthetic cytokine receptor complex activated by a non-physiological ligand, and differentiated cells produced from those stem or progenitor cells for use in medical treatment. The differentiated cells may be, but are not limited to, iCIL cells. Advantages of embodiments may include the ability to generate for a plentiful cell source (e.g., induced pluripotent stem cells) effector cells expressing synthetic cytokine receptor complex activated by a non-physiological ligand, so that proliferation of the effector cells in patients may be controlled by administering or ceasing administration of the non-physiological ligand. Other advantages of embodiments include, but are not limited to, the ability to generate effector cells from source cells in media substantially free of cytokines conventionally used in the art for CIL cell differentiation, such as IL-2, IL-7, and/or IL-15.

CIL cells may be generated from multiple sources, illustrative examples include: iPSCs, PBMCs, or UCBs. In some embodiments, the CIL source cells are autologous cells. In some embodiments, the CIL source cells are allogeneic cells. In some embodiments, the CIL source cells are heterologous cells. For example, when the subject being treated using the compositions of the present disclosure has received high-dose chemotherapy or radiation treatment to destroy the subject's immune system, allogenic cells may be used.

In some embodiments, hematopoietic stem cells may be engineered to express a synthetic cytokine receptor. In some embodiments, blood progenitor cells (leukocytes) cells may be engineered to express a synthetic cytokine receptor. In some embodiments, common lymphoid progenitor cells may be engineered to express a synthetic cytokine receptor. In some embodiments, CIL cells may be engineered to express a synthetic cytokine receptor.

In some embodiments, the non-physiological ligand may induce differentiation, in addition to or instead of an exogenous cytokine. In some embodiments, the non-physiological ligand may induce differentiation during one or more of mesoderm formation, hematopoietic specification, lymphoid progenitor differentiation, CIL cell differentiation.

In some embodiments, the non-physiological ligand may be contacted with cells in a differentiation phase requiring an IL-7 and/or IL-15 signal.

In some embodiments, the non-physiological ligand may be contacted with cells in an expansion phase requiring an IL-2 signal.

In some embodiments, engineering cells to express a synthetic cytokine receptor and activating the receptor with a non-physiological ligand allows for the generation of CIL cells that may be differentiated and/or expanded without the use of exogenous factors.

Because it is well-known in the relevant art that differentiation and/or expansion of CIL cells depends on the presence of various exogenous stimuli, the differentiation and/or expansion of the engineered CIL cells without one, two, or all of IL-2, IL-15, and IL-7, as described herein, is surprising and unexpected.

In some embodiments, engineered common lymphoid progenitors are differentiated into CIL cells without IL-15 in the differentiation medium.

In some embodiments, engineered common lymphoid progenitors are differentiated into CIL cells without IL-7 in the differentiation medium.

In some embodiments, engineered common lymphoid progenitors are differentiated into CIL cells without IL-15 or IL-7 in the differentiation medium.

In some embodiments, common lymphoid progenitors are engineered to express a synthetic cytokine receptor, such as RACR, and differentiated into CIL cells with a rapalog in the differentiation medium and without IL-15 or IL-7 in the differentiation medium.

In some embodiments, engineered CIL cells are expanded without IL-2 in the expansion medium.

In some embodiments, CIL cells are engineered to express a synthetic cytokine receptor, such as RACR, and expanded with a rapalog in the expansion medium and without recombinant cytokines in the expansion medium.

In some embodiments, the CIL cell expanding step is performed in cell culture media that is substantially free of recombinant cytokines.

In some embodiments, the expanding step is performed in a feeder-free cell culture.

In some embodiments, the CIL cell expanding step is performed in culture vessels not coated with recombinant ligands for CIL cell expansion.

In some embodiments, provided is a method of administering an effective amount of non-physiological ligand to the subject, wherein the non-physiological ligand causes the common lymphoid progenitor cells to differentiate to CIL cells according to any of the foregoing embodiments.

In some embodiments, provided is a method of administering an effective amount of non-physiological ligand to the subject, wherein the non-physiological ligand causes the cells to proliferate according to any of the foregoing embodiments.

In some embodiments, the non-physiological ligand is present or provided at an amount effective to cause expansion of the CIL cells ex vivo.

In some embodiments, the non-physiological ligand is present or provided at an amount effective to cause CIL cell cytokine secretion ex vivo.

In some embodiments, the non-physiological ligand is present or provided at an amount effective to cause CIL cell secretion of CD107a, interferon gamma (IFNγ) and/or tumor necrosis factor alpha (TNF-α) ex vivo.

In some embodiments, the non-physiological ligand is present or provided at an amount effective to cause tumor cell killing.

In some embodiments, the non-physiological ligand is present or provided at a therapeutically effective amount.

IX. Methods of Treating Subjects with the Disclosed Compositions

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed cells to the subject. Also provided is a method of treating a tumor and/or killing tumor cells in a subject, comprising administering an effective amount of non-physiological ligand to the subject, wherein the non-physiological ligand causes the cells to proliferate according to any of the foregoing embodiments.

In some embodiments, the malignancy is a solid tumor, sarcoma, carcinoma, lymphoma, multiple myeloma. Hodgkin's Disease, non-Hodgkin's lymphoma (NHL), primary mediastinal large B cell lymphoma (PBMC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitf's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be used to treat cancer and/or kill cancer cells in a subject by administering a therapeutically effective amount of the cells according to any of the foregoing embodiments.

The present disclosure also provides a method of treating cancer and/or killing cancer cells in a subject, comprising administering the system of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, the target cell is a tumor cell. In some embodiments, the target cell exists in a tumor microenvironment.

In some embodiments, the present disclosure provides a method of treating cancer with any of the compositions provided herein without prior conditioning of the subject. In some embodiments, a subject of the present disclosure does not need to receive a lymphodepleting therapy. A person skilled in the art will understand the common lymphodepleting therapies available, such as chemotherapy. In some embodiments, a subject of the present disclosure has not received a lymphodepleting therapy. In some embodiments, a differentiated cell is provided to a subject that has not received a lymphodepleting therapy. In some embodiments, the subject has not received a lymphodepleting therapy for 1, 2, 3, 4, 5, 6, 7, 8, 9 or 10 days prior to administration of the differentiated cell.

In some embodiments, a differentiated cell is provided to the subject 1, 2, 3, 4, 5, 6, 7, 8, 9, 10, 11, 12, 15, 20, 24, 36, 48, 60 or 72 hours after administration of a ligand composition, or any time within a range defined by any two aforementioned values. In some embodiments, the cell is provided to the subject 1, 2, 3, 4, 5, 6, 7, 8, 9, 10, 11, 12, 15, 20, 24, 36 or 48 hours before administration of the ligand composition, or any time within a range defined by any two aforementioned values. In some embodiments, the cell is provided to the subject within seconds or minutes, such as less than an hour, of providing the composition to the subject. In some embodiments, a boost of the cell and/or the composition is provided to the subject.

In some aspects, the present disclosure provides a method of treating a cancer as described in WO 2019/144095, which is incorporated herein by reference in its entirety.

In some embodiments, the present disclosure provides a method of treating cancer comprising administering a chimeric antigen receptor cell (e.g., an NK cell), wherein the CAR comprises an E2 anti-fluorescein antibody fragment. In some embodiments, the method of treating cancer further comprises administering a small molecule linked to a targeting moiety by a linker.

In some embodiments, the targeting moiety is determined by the type of cancer being treated in the subject. As an example, folate receptor is highly expressed on the surface of a wide variety of solid tumor cells including breast (e.g., triple negative breast cancer), ovarian, endometrial, kidney, lung, brain, pancreatic, gastric, prostate, acute myelocytic leukemia, and non-small cell lung cancers. Thus, in some embodiments, the targeting moiety comprises a folate, which would bind folate receptor expressed on a cancer or tumor cell. In other embodiments, the folate can be folic acid, a folic acid analog or any folate-receptor binding molecule. In some embodiments, the small molecule comprises fluorescein, fluorescein isothiocyanate (FITC), NHS-fluorescein, or any other fluorophore. In some embodiments, the small molecule linked to a targeting moiety is FITC-Folate.

In some embodiments, the disclosure contemplates engraftment of engineered stem cells for use in the treatment of subjects with cancer. In some embodiments, the iPSC-derived engineered cytotoxic innate lymphoid cells stably engraft in the subject. In some embodiments, the iPSC-derived engineered cytotoxic innate lymphoid cells display long-term engraftment in the subject. In some embodiments, administration of the iPSC-derived engineered cytotoxic innate lymphoid cells allowed for engraftment and further differentiation within the subject. In some embodiments, the iPSC-derived engineered cytotoxic innate lymphoid cells are natural killer cells.

In some embodiments, an additional cancer therapy is provided, such as a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T. Sorafenib, Sunitinib, Talc Intrapleural, Tamoxifen, Temozolomide. Temsirolimus, Thalidomide, Trastuzumab, Vinorelbine or Zoledronic acid.

### Modes of Administration and Dosing

In some embodiments, transduced lymphocytes, such as CIL cells, engineered with a synthetic cytokine receptor as described herein may be grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma. In some embodiments, any of the cell culturing methods contemplated in the disclosure may be used to grow CAR-expressing lymphocytes, such a as CAR-expressing CIL cells.

In some embodiments, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation either with or without the agent that was used to promote activation. In some embodiments, any of the methods of administering the engineered lymphocytes, such as CIL cells, contemplated in the disclosure may be used to administer CAR-expressing lymphocytes, such as CAR-expressing CIL cells to the patient.

The disclosed cells may be administered in a number of ways depending upon whether local or systemic treatment is desired.

In the case of adoptive cell therapy, methods for administration of cells for adoptive cell therapy are known and may be used in connection with the provided methods and compositions.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In an embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Illustrative parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing differentiated cells or transgenic differentiated cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

### X. EXEMPLARY EMBODIMENTS

Among the provided embodiments are:

1. A cell population comprising engineered cytotoxic innate lymphoid cells engineered for controlled expansion and/or activity, the engineered cytotoxic innate lymphoid cells comprising a synthetic cytokine receptor for a non-physiological ligand,
wherein the cytokine receptor comprises:
   a synthetic gamma chain polypeptide comprising a first dimerization domain, a first transmembrane domain, and an interleukin-2 receptor subunit gamma (IL-2RG) intracellular domain, and
   a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain;
wherein the non-physiological ligand activates the synthetic cytokine receptor in the cytotoxic innate lymphoid cells to induce expansion and/or activation of the engineered cytotoxic innate lymphoid cells.

2. The cell population of embodiment 1, wherein the beta chain intracellular domain comprises the IL-2RB intracellular domain.

3. The cell population of embodiment 2, wherein the IL-2RB intracellular domain comprises a polypeptide sequence at least 95% identical to SEQ ID NO: 2, or a polypeptide sequence as set forth in SEQ ID NO: 2.

4. The cell population of embodiment 1, wherein the beta chain intracellular domain comprises the IL-7RB intracellular domain.

5. The cell population of embodiment 4, wherein the IL-7RB intracellular domain comprises a polypeptide sequence at least 95% identical to SEQ ID NO: 3, or a polypeptide sequence as set forth in SEQ ID NO: 3.

6. The cell population of embodiment 1, wherein the beta chain intracellular domain comprises the IL-21RB intracellular domain.

7. The cell population of embodiment 6, wherein the IL-21RB intracellular domain comprises a polypeptide sequence at least 95% identical to SEQ ID NO: 4, or a polypeptide sequence as set forth in SEQ ID NO: 4.

8. The cell population of any one of embodiments 1 to 7, wherein the first dimerization domain and the second dimerization domain are extracellular domains;
the synthetic gamma chain polypeptide comprises, in N- to C-terminal order, the first dimerization domain, the first transmembrane domain, and the interleukin-2 receptor subunit gamma (IL-2RG) intracellular domain, and
the synthetic beta chain polypeptide comprises, in N- to C-terminal order, the second dimerization domain, the second transmembrane domain, and the intracellular domain.

9. The cell population of any one of embodiments 1 to 8, wherein the first dimerization domain and the second dimerization domain are heterodimerization domains selected from selected from FK506-Binding Protein of size 12 kD (FKBP) and a FKBP12-rapamycin binding (FRB) domain; and/or
wherein the non-physiological ligand is rapamycin or a rapalog.

10. The cell population of embodiment 9, wherein the FRB domain comprises a polypeptide sequence at least 95% identical to SEQ ID NO: 6 or SEQ ID NO: 7.

11. The cell population of embodiment 9, wherein the FRB domain comprises the polypeptide sequence set forth in SEQ ID NO: 6 or SEQ ID NO: 7.

12. The cell population of any one of embodiments 1 to 8, wherein the first dimerization domain and the second dimerization domain are heterodimerization domains selected from selected from FK506-Binding Protein of size 12 kD (FKBP) and a calcineurin domain; and/or
wherein the non-physiological ligand is FK506 or an analogue thereof.

13. The cell population of any one of embodiments 1 to 12, wherein the FKBP domain comprises a polypeptide sequence at least 95% identical to SEQ ID NO: 5.

14. The cell population of any one of embodiments 1 to 12, wherein the FKBP domain comprises the polypeptide sequence set forth in SEQ ID NO: 5.

15. The cell population of any one of embodiments 1 to 8, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
   and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

16. The cell population of any one of embodiments 1 to 15, wherein the cytotoxic innate lymphoid cells express a cytosolic polypeptide that binds to the non-physiological ligand.

17. The cell population of any one of embodiments 1 to 15, wherein the non-physiological ligand is rapamycin or a rapalog, and the cytotoxic innate lymphoid cells express a cytosolic FRB domain or variant thereof.

18. The cell population of embodiment 17, wherein the cytosolic FRB domain comprises a polypeptide sequence at least 95% identical to SEQ ID NO: 6 or SEQ ID NO: 7.

19. The cell population of embodiment 17, wherein the cytosolic FRB domain comprises a polypeptide sequence at least 98% identical to SEQ ID NO: 6 or SEQ ID NO: 7.

20. The cell population of any one of embodiments 1 to 19, wherein the cytotoxic innate lymphoid cells are primary cytotoxic innate lymphoid cells.

21. The cell population of any one of embodiments 1 to 19, wherein the cytotoxic innate lymphoid cells are blood-derived cytotoxic innate lymphoid cells.

22. The cell population of any one of embodiments 1 to 19, wherein the cytotoxic innate lymphoid cells are induced cytotoxic innate lymphoid (iCIL) cells.

23. The cell population of any one of embodiments 1 to 22, wherein the engineered cells are CD3−, CD5−, CD16+, CD56+, CD57+, NKp30+, NKp46+, NKG2A+, and/or NKG2D+.

24. The cell population of any one of embodiments 1 to 23, wherein the population of engineered cytotoxic innate lymphoid cells is at least 40% CD16+.

25. The cell population of any one of embodiments 1 to 24, wherein the population of engineered cytotoxic innate lymphoid cells is or at least 40% CD56+.

26. The cell population of any one of embodiments 1 to 23, wherein the population of engineered cytotoxic innate lymphoid cells is at least 40% CD16+CD56+.

27. The cell population of any one of embodiments 1 to 26, wherein the engineered cytotoxic innate lymphoid cells express CD16, CD56, CD57, NKG2A, NKG2D, and/or CD57 at least the same level as control cytotoxic innate lymphoid cells expanded with IL-2.

28. The cell population of any one of embodiments 1 to 27, wherein the engineered cytotoxic innate lymphoid cells express NKp30 and/or NKp46 at higher levels than control cytotoxic innate lymphoid cells expanded with IL-2.

29. The cell population of any one of embodiments 1 to 28, wherein the engineered cytotoxic innate lymphoid cells secrete CD107a in response to an antigen recognized by the engineered cytotoxic innate lymphoid cells.

30. The cell population of any one of embodiments 1 to 29, wherein the engineered cytotoxic innate lymphoid cells secrete interferon gamma (IFNγ) and/or tumor necrosis factor alpha (TNF-α) in response to an antigen recognized by the engineered cytotoxic innate lymphoid cells.

31. A method of making engineered cytotoxic innate lymphoid cells for controlled expansion and/or activity, comprising:
contacting source cells with a vector comprising a polynucleotide encoding a synthetic cytokine receptor for a non-physiological ligand,
thereby transducing the source cells to generate a cell population comprising transduced cells;
   wherein the cytokine receptor comprises:
      a synthetic gamma chain polypeptide comprising a first dimerization domain, a first transmembrane domain, and an interleukin-2 receptor subunit gamma (IL-2RG) intracellular domain, and
      a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; and
expanding engineered cytotoxic innate lymphoid cells in the cell population by contacting the cell population with the non-physiological ligand to activate the synthetic cytokine receptor,
thereby causing expansion of engineered cytotoxic innate lymphoid cells in the cell population.

32. The method of embodiment 31, wherein the source cells are primary cytotoxic innate lymphoid cells.

33. The method of embodiment 31, wherein the source cells are hematopoietic progenitor cells or hematopoietic stem cells or common lymphoid progenitor cells, obtained by differentiating human pluripotent stem cells or human embryonic stem cells.

34. The method of embodiment 31, wherein the source cells are human pluripotent stem cells.

35. The method of embodiment 33 or embodiment 34, wherein the method comprises contacting the cell population with the non-physiological ligand during differentiation of the transduced cells into engineered cytotoxic innate lymphoid cells.

36. The method of any one of embodiments 31 to 35, wherein the expanding step is performed in cell culture media that is substantially free of recombinant cytokines; wherein the expanding step is performed in a feeder-free cell culture; and/or wherein the expanding step is performed in culture vessels not coated with recombinant ligands for cytotoxic innate lymphoid cell expansion.

37. A cell population produced by the method of any one of embodiments 31 to 36.

38. A pharmaceutical composition comprising the cell population of any one of embodiments 1 to 30 or embodiment 37.

39. A method of immunotherapy for a subject in need thereof, comprising administering to the subject an effective amount of the cell population of any one of embodiments 1 to 30 or embodiment 37, or the pharmaceutical composition of embodiment 38.

40. The method of embodiment 39, comprising administering to the subject the non-physiological ligand in an amount effective to cause the engineered cytotoxic innate lymphoid cells to expand in the subject.

41. The method of embodiment 39 or embodiment 40, comprising administering to the subject the non-physiological ligand in an amount effective to cause the engineered cytotoxic innate lymphoid cells to present CD107a, or secrete interferon gamma (IFNγ) and/or tumor necrosis factor alpha (TNFα).

42. The method of any one of embodiments 39 to 41, comprising administering to the subject the non-physiological ligand in an amount effective to cause the engineered cytotoxic innate lymphoid cells to kill tumor cells.

43. A kit comprising the cell population of any one of embodiments 1 to 30 or embodiment 37, or the pharmaceutical composition of embodiment 38, and instructions for use.

44. A cell population comprising engineered cells engineered for controlled expansion and/or activity, the engineered cells comprising a synthetic cytokine receptor for a non-physiological ligand,
wherein the cytokine receptor comprises:
   a synthetic gamma chain polypeptide comprising a first dimerization domain, a first transmembrane domain, and an interleukin-2 receptor subunit gamma (IL-2RG) intracellular domain, and
   a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain;
wherein the non-physiological ligand activates the synthetic cytokine receptor in the engineered cells to induce expansion and/or activation of the engineered cells.

45. The cell population of embodiment 44, wherein the engineered cells are human pluripotent stem cells.

46. The cell population of embodiment 44, wherein the engineered cells are hematopoietic stem cells or hematopoietic progenitor cells.

47. The cell population of embodiment 44, wherein the engineered cells are common lymphoid progenitor cells.

48. The cell population of any one of embodiments 44 to 47, wherein the cell population is a cell population of any one of embodiments 2 to 30 or embodiment 37.

49. The pharmaceutical composition or kit comprising the cell population of any one of embodiments 44 to 48.

50. A method of making the cell population of embodiment 1 according to the method of any one of embodiments 31 to 37.

51. Use of the cell population of embodiment 50 in a method according to any one of embodiments 38 to 42.

52. A method of making and/or expanding a population of engineered cells comprising a synthetic cytokine receptor for a non-physiological ligand, the method comprising:
a) providing engineered cells comprising a synthetic cytokine receptor, optionally by introducing into source cells a polynucleotide encoding a synthetic cytokine receptor, and
b) incubating the engineered source cells in media comprising a non-physiological ligand,
   wherein the cytokine receptor comprises:
      a synthetic gamma chain polypeptide comprising a first dimerization domain, a first transmembrane domain, and an interleukin-2 receptor subunit gamma (IL-2RG) intracellular domain, and
      a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain;
wherein the non-physiological ligand activates the synthetic cytokine receptor in the engineered cells to induce expansion and/or activation of the engineered cells.

53. The method of embodiment 52, wherein the source cells are induced pluripotent stem cells.

54. The method of embodiment 52, wherein the source cells are hematopoietic stem cells or hematopoietic progenitor cells.

55. The method of embodiment 52, wherein the source cells are common lymphoid progenitor cells.

56. The method of embodiment 52, wherein the source cells are cytotoxic innate lymphoid cells.

57. The method of embodiment 52, wherein the vector is a lentiviral vector.

58. The method of embodiment 52, wherein the non-physiological ligand is a rapalog.

59. An engineered cytotoxic innate lymphoid (CIL) cell engineered for controlled expansion and/or activity, the engineered CIL cell comprising a synthetic cytokine receptor for a non-physiological ligand,
wherein the cytokine receptor comprises:
   a synthetic gamma chain polypeptide comprising a first dimerization domain, a first transmembrane domain, and an interleukin-2 receptor subunit gamma (IL-2RG) intracellular domain, and
   a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain;
wherein binding of the non-physiological ligand to the synthetic cytokine receptor activates the synthetic cytokine receptor in the engineered CIL cell to induce expansion and/or activation of the engineered CIL cell in a cell population.

60. The engineered CIL cell of embodiment 59, wherein the first dimerization domain and the second dimerization domain are extracellular domains.

61. The engineered CIL cell of embodiment 59 or embodiment 60, wherein the synthetic gamma chain polypeptide comprises, in N- to C-terminal order, the first dimerization domain, the first transmembrane domain, and the interleukin-2 receptor subunit gamma (IL-2RG) intracellular domain, and the synthetic beta chain polypeptide comprises, in N- to C-terminal order, the second dimerization domain, the second transmembrane domain, and the intracellular domain.

62. The engineered CIL cell of any of embodiments 59-61, wherein the IL-2RG intracellular domain comprises a polypeptide sequence at least 95% identical to SEQ ID NO: 1, or a polypeptide sequence as set forth in SEQ ID NO: 1.

63. The engineered CIL cell of any of embodiments 59-62, wherein the first transmembrane domain comprises the IL-2RG transmembrane domain.

64. The engineered CIL cell of embodiment 63, wherein the IL-2RG transmembrane domain comprises a polypeptide sequence at least 95% identical to SEQ ID NO: 8 or 31, or a polypeptide sequence as set forth in SEQ ID NO: 8 or 31.

65. The engineered CIL cell of any of embodiments 59-64, wherein the beta chain intracellular domain comprises the IL-2RB intracellular domain.

66. The CIL cell of embodiment 65, wherein the IL-2RB intracellular domain comprises a polypeptide sequence at least 95% identical to SEQ ID NO: 2, or a polypeptide sequence as set forth in SEQ ID NO: 2.

67. The engineered CIL cell of any of embodiments 59-64, wherein the beta chain intracellular domain comprises the IL-7RB intracellular domain.

68. The engineered CIL cell of any of embodiments 59-64, wherein the IL-7RB intracellular domain comprises a polypeptide sequence at least 95% identical to SEQ ID NO: 3, or a polypeptide sequence as set forth in SEQ ID NO: 3.

69. The engineered CIL cell of any of embodiments 59-64, wherein the beta chain intracellular domain comprises the IL-21RB intracellular domain.

70. The engineered CIL cell of embodiment 69, wherein the IL-21RB intracellular domain comprises a polypeptide sequence at least 95% identical to SEQ ID NO: 4, or a polypeptide sequence as set forth in SEQ ID NO: 4.

71. The engineered CIL cell of any of embodiments 59-70, wherein the second transmembrane domain comprises a transmembrane domain from the same beta chain intracellular domain.

72. The engineered CIL cell of embodiment 59-66 and 71, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

73. The engineered CIL cell of any of embodiments 59-66, 71 and 72, wherein:
the synthetic gamma chain polypeptide contains an IL-2RG TM domain comprising the sequence set forth in SEQ ID NO: 8 or 31 and an IL-2RG intracellular domain comprising the sequence set forth in SEQ ID NO:1; and the synthetic beta chain polypeptide contains an IL-2RB TM domain comprising the sequence set forth in SEQ ID NO: 35 or 36 and an IL-2RB intracellular domain comprising the sequence set forth in SEQ ID NO:2.

74. The engineered CIL cell of any of embodiments 59-73, wherein the first dimerization domain and the second dimerization domain are heterodimerization domains selected from selected from FK506-Binding Protein of size 12 kD (FKBP) and a FKBP12-rapamycin binding (FRB) domain; and/or wherein the non-physiological ligand is rapamycin or a rapalog.

75. The engineered CIL cell of embodiment 74, wherein the FRB domain comprises a polypeptide sequence at least 95% identical to SEQ ID NO: 6 or SEQ ID NO: 7.

76. The engineered CIL cell of embodiment 74, wherein the FRB domain comprises the polypeptide sequence set forth in SEQ ID NO: 6 or SEQ ID NO: 7.

77. The engineered CIL cell of any of embodiments 59-73, wherein the first dimerization domain and the second dimerization domain are heterodimerization domains selected from selected from FK506-Binding Protein of size 12 kD (FKBP) and a calcineurin domain; and/or
wherein the non-physiological ligand is FK506 or an analogue thereof.

78. The engineered CIL cell of any of embodiments 59-74, wherein the FKBP domain comprises a polypeptide sequence at least 95% identical to SEQ ID NO: 5 or SEQ ID NO: 30.

79. The engineered CIL cell of any of embodiments 59-74, wherein the FKBP domain comprises the polypeptide sequence set forth in SEQ ID NO: 5 or SEQ ID NO: 30.

80. The engineered CIL cell of any of embodiments 59-66 and 71-79, wherein the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

81. The engineered CIL cell of any of embodiments 59-66 and 71-80, wherein the synthetic gamma chain polypeptide has the sequence of amino acids set forth in SEQ ID O: 28 and the synthetic beta chain polypeptide has the sequence of amino acids set forth in SEQ ID NO:33.

82. The engineered CIL cell of any of embodiments 59-73, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
   and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

83. The engineered CIL cell of any of embodiments 59-82, wherein the CIL cell is resistant to rapamycin-mediated mTOR inhibition.

84. The engineered CIL cell of any of embodiments 59-83, wherein the cytotoxic innate lymphoid cells express a cytosolic polypeptide that binds to the non-physiological ligand.

85. The engineered CIL cell of any of embodiments 59-84, wherein the non-physiological ligand is rapamycin or a rapalog, and the cytotoxic innate lymphoid cells express a cytosolic FRB domain or variant thereof.

86. The engineered CIL cell of embodiment 85, wherein the cytosolic FRB domain comprises a polypeptide sequence at least 95% identical to SEQ ID NO: 6 or SEQ ID NO: 7.

87. The engineered CIL cell of embodiment 85, wherein the cytosolic FRB domain comprises a polypeptide sequence at least 98% identical to SEQ ID NO: 6 or SEQ ID NO: 7.

88. The engineered CIL cell of any of embodiments 59-87, wherein the CIL cell comprises a disrupted FKBP12 gene that reduces expression of FKBP12.

89. The engineered CIL cell of any of embodiments 59-88, wherein the CIL cell comprises knock out of the FKBP12 gene.

90. The engineered CIL cell of any of embodiments 59-89, wherein the CIL cells comprise a nucleotide sequence encoding the synthetic cytokine receptor inserted into the genome of the CIL cell

91. The engineered CIL cell of embodiment 90, wherein the nucleotide sequence encoding the synthetic cytokine receptor is inserted into a non-target locus in the genome of the CIL cell.

92. The engineered CIL cell of embodiment 90, wherein the nucleotide sequence encoding the synthetic cytokine receptor is inserted into an endogenous gene of the CIL cell.

93. The engineered CIL cell of embodiment 92, wherein the insertion reduces expression of the endogenous gene in the locus.

94. The engineered CIL cell of embodiment 92 or embodiment 93, wherein the insertion knocks out the endogenous gene in the locus.

95. The engineered CIL cell of any of embodiments 92-94 wherein the insertion is by homology-directed repair.

96. The engineered CIL cell of any of embodiments 92-95, wherein the endogenous gene is a housekeeping gene, a blood-lineage specific loci or an immune-related gene.

97. The engineered CIL cell of embodiment 96, wherein endogenous gene is a housekeeping gene and the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).

98. The engineered CIL cell of embodiment 96, wherein the endogenous gene is a blood-lineage specific loci and the blood-lineage specific loci is selected from protein tyrosine phosphatase receptor type C (PTPRC), IL2RG, and IL2RB.

99. The engineered CIL cell of embodiment 96, wherein the immune-related gene is selected from a beta-2-microglobulin (B2M) gene, a T cell receptor alpha constant (TRAC) gene, and a signal regulatory protein alpha (SIRPA) gene.

100. The engineered CIL cell of any one of embodiments 59-99, wherein the CIL cells comprise a B2M knockout.

101. The engineered CIL cell of any of embodiments 59-100, wherein the CIL cells comprise a B2M knockout and a FKBP12 knockout.

102. The engineered CIL cell of any one of embodiments 59-101, comprising a chimeric antigen receptor (CAR).

103. The engineered CIL cell of embodiment 102, wherein the CAR is an anti-FITC CAR.

104. A cell population comprising engineered CIL cells of any of embodiments 59-103.

105. A method of genetically engineering CIL cells to express a synthetic cytokine receptor, comprising:
contacting a population of CIL cells with (i) a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand,
thereby inserting the nucleotide sequence into the endogenous gene;
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

106. The method of embodiment 105, wherein the nucleotide sequence is inserted via homology directed repair (HDR).

107. The method of embodiment 106, wherein the vector comprises a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream.

108. The method of embodiment 105, wherein the nucleotide sequence is inserted via non-homologous end joining (NHEJ).

109. The method of any one of embodiments 105-108, wherein the RNA-guided endonuclease is selected from a Cas endonuclease, a Mad endonuclease, and a Cpf1 endonuclease.

110. The method of any one of embodiments 105-109, wherein the RNA-guided endonuclease is Cas9 or Mad7.

111. The method of any of embodiments 105-110, wherein the endogenous gene is selected from B2M, TRAC and SIRPA.

112. The method of any of embodiments 105-111, wherein the endogenous gene is B2M.

113. The method of any of embodiments 105-112, wherein the gRNA comprises the sequence set forth in SEQ ID NO:18.

114. The method of any of embodiments 107-113, wherein the nucleotide sequence homologous with a region located upstream of the target site comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the nucleotide sequence homologous with a region located downstream comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23.

115. The method of any of embodiments 105-114, wherein the nucleotide sequence encoding the synthetic cytokine receptor comprises a first nucleic acid sequence encoding a gamma chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37, and a second nucleic acid sequence encoding a beta chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38.

116. The method of embodiment 115, wherein the first nucleic acid sequence and second nucleic acid sequence are separated by a cleavable linker or an IRES.

117. The method of embodiment 116, wherein the cleavable linker is a protein quantitation reporter linker (PQR), optionally set forth in SEQ ID NO:42.

118. The method of any of embodiments 105-127, wherein the nucleotide sequence encoding a synthetic cytokine receptor for a non-physiological ligand is under the operable control of a heterologous promoter.

119. The method of embodiment 118, wherein the heterologous promoter is the EF1a promoter or the MND promoter.

120. The method of any of embodiments 105-118, wherein the nucleotide sequence encoding the synthetic cytokine receptor comprises a polyadenylation sequence.

121. The method of any of embodiments 105-120, wherein the recombinant vector comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40.

122. The method of any one of embodiments 105-111, comprising engineering the population of CIL cells to be resistant to rapamycin-mediated mTOR inhibition.

123. The method of embodiment 122, wherein engineering the population of CIL cells to be resistant to rapamycin comprises knocking out a FKBP12 gene.

124. The method of embodiment 122, wherein the method further comprising contacting the population of CIL cells with a guide RNA (gRNA) targeting a target site in the FKBP12 gene.

125. The method of embodiment 122, wherein the gRNA comprises one or more gRNA selected from a gRNA comprising the sequence set forth in SEQ ID NO: 19, SEQ ID NO:20 or SEQ ID NO:21.

126. The method of embodiment 125, wherein the gRNA is a pool of gRNA comprising 2 or 3 gRNA.

127. The method of any of embodiments 105-126, further comprising introducing into the population of CIL cells a chimeric antigen receptor (CAR).

128. The method of embodiment 127, wherein the CAR is an anti-FITC CAR.

129. A cell population produced by the method of any one of embodiments 105-128.

130. A pharmaceutical composition comprising the cell population of any one of embodiments 59-90 and 129.

131. A method of expanding an engineered cytotoxic innate lymphoid cell (CIL), the method comprising contacting a CIL of any of embodiments 59-103 with the non-physiological ligand of the synthetic cytokine receptor.

132. A method of treating a cancer in a subject, comprising administering to the subject an effective amount of the engineered CIL of any one of embodiments 59-103, the cell population of embodiment 129, or the pharmaceutical composition of embodiment 130.

133. A method of treating a cancer in a subject, comprising administering to the subject an effective amount of the engineered CIL of any of embodiments 59-103, the cell population of embodiment 129, or the pharmaceutical composition of embodiment 130.

134. The method of embodiment 133, wherein the subject has not been administered a lymphodepleting therapy prior to the administering the CIL, population of CILs or the pharmaceutical composition.

135. The method of embodiment 133 or embodiment 134, wherein the CIL express a CAR targeting cancer cells in the subject.

136. The method of embodiment 135, wherein the CAR is an anti-FITC CAR and the subject has been administered a FITC-ligand to tag a cancer cell in the subject, wherein the ligand specifically binds a molecule expressed on a tumor. 137. The method of embodiment 136, wherein the FITC-ligand is FITC-folate.

138. The method of embodiment 137, wherein the non-physiological ligand is rapamycin or a rapamycin analog, optionally wherein the rapamycin analog is rapalog.

139. The method of any of embodiments 131-138, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

140. The method of any of embodiments 131-138, wherein the non-physiological ligand is contacted at a concentration of at or about 10 nM.

141. The method of any of embodiments 131-138, wherein the non-physiological ligand is contacted at a concentration of at or about 100 nM 142. A method of killing or inhibiting the proliferation of cancer cells, comprising contacting cancer cells with the engineered CIL of any of embodiments 59-103, the cell population of embodiment 129, or the pharmaceutical composition of embodiment 130.

143. The method of killing or inhibiting the proliferation of cancer cells of embodiment 142, wherein the method is performed in vivo in a subject.

144. A kit comprising the engineered CIL of any of embodiments 59-103, the cell population of embodiment 129, or the pharmaceutical composition of embodiment 130 and instructions for administering to a subject in need thereof.

145. The kit of embodiment 144, further comprising a container comprising the non-physiological ligand and instructions for administering the non-physiological ligand to the subject after administration of the cell population.

146. The kit of embodiment 144 or 145, wherein the subject has a cancer.

147. The method of any of embodiments 142-146, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.

### Examples

The following examples are put forth so as to provide those of ordinary skill in the art with a description of how the compositions and methods described herein may be used, made, and evaluated, and are intended to be purely illustrative of the invention and are not intended to limit the scope of what is regarding as the invention.

### Example 1: RACR-Expansion of Highly Functional CD19CAR Blood-Derived NK Cells

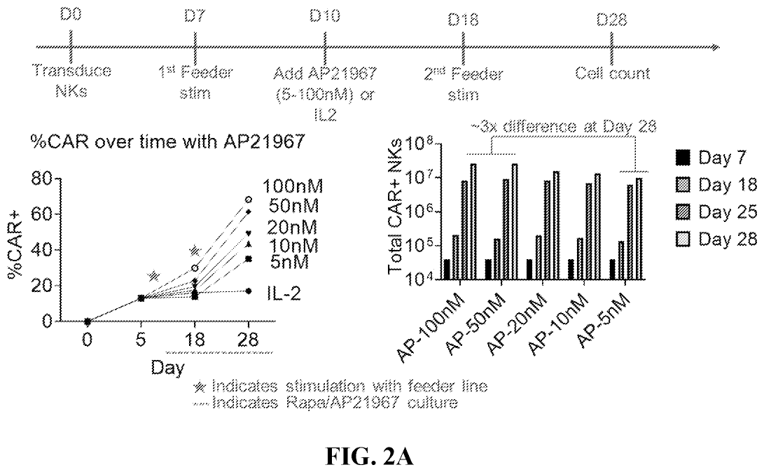

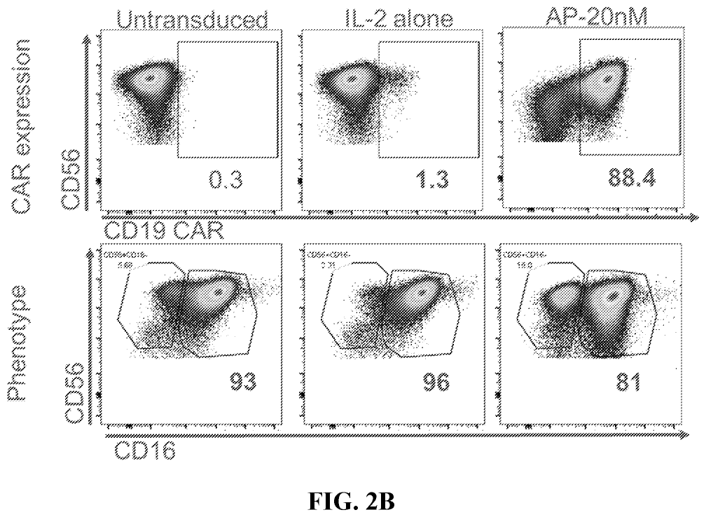

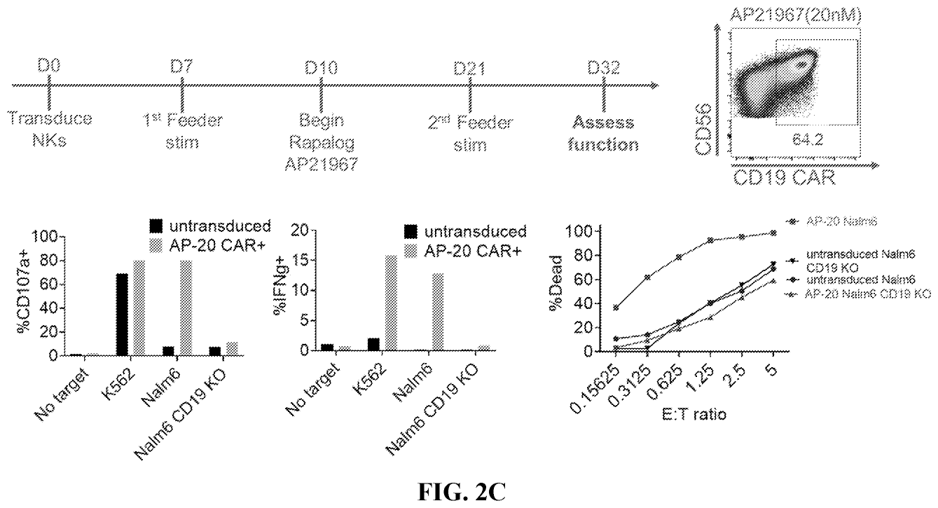

This example demonstrates RACR function in fully differentiated blood-derived NK cells (bdNK). This example also demonstrates that the RACR signal can drive NK cell expansion. A timeline of the experiment is shown in FIG. 2A.

The RACR system is a synthetic cytokine receptor that is composed of a synthetic gamma chain polypeptide containing a first dimerization domain (e.g. FKBP or FRB), a transmembrane domain and an interleukin-2 receptor subunit gamma (IL-2RG, SEQ ID NO: 1) chain, and a synthetic beta chain polypeptide containing a second dimerization domain (e.g. the other of FKBP and FRB), a transmembrane domain, and an intracellular domain from a cytokine receptor having a beta chain (e.g. IL-2RB, SEQ ID NO: 2). An exemplary sequence of a RACR is encoded by a polynucleotide set forth in SEQ ID NO: 32. NK cells were co-engineered with a polynucleotide encoding the RACR synthetic cytokine receptor and a polynucleotide encoding a CD19-directed CAR containing an FMC63-derived antigen-binding domain and an intracellular signaling domain containing a 4-1BB intracellular signaling domain and a CD3zeta signaling domain.

On Day-2, NK cells were isolated from peripheral blood mononuclear cells (PBMCs) using the Biolegend® human NK isolation kit and incubated in RPMI media containing 10% FBS, Glutamax, Pen-strep, IL-2 (1000 U/ml), IL-21 (20 ng/ml), and Rosuvastatin® (5 μM) for two days. On Day 0, the cells were washed in RPMI media and transduced with CD19-RACR/VSVG lentiviral vector at an MOI of 30 in the presence of IL-2 (1000 U/ml), IL-21 (20 ng/ml), BX795 (6 μM), and protamine sulfate (8 μg/ml). Three days later, the media was replaced with fresh RPMI media containing IL-2 (1000 U/ml) and IL-21 (20 ng/ml). On Day 5, cells were assessed for CD19 CAR expression by flow cytometry. On Day 7, cells were counted using the TC-20 cell counter (Biorad®) and stimulated with irradiated (10,000 rads) K562 cells expressing membrane-bound IL-21 and 41BBL at a ratio of ˜10 feeder K562 cells:1 NK cell. On Day 10, the cells were washed and resuspended in fresh RPMI media containing either IL-2 (1000 U/ml) or varying concentrations of AP21967 (5-100 nM) (A/C Heterodimerizer-Takara®). On Day 18, cells were counted and CAR expression was assessed by flow cytometry and the cells were stimulated again with K562 feeder cells at a ratio of 10 feeder cells:1 NK cell in RPMI media containing either IL-2 or AP21967 using the same concentrations as on Day 10. The cells were cultured for an additional 10 days, counted, and CAR expression was analyzed using flow cytometry. As seen in FIG. 2A, percent CAR expression over time for each group is shown in the left panel and total CAR+ NK cells over time are shown in the right panel. RACR-NK cells showed dose-responsive AP21967-driven feeder-based expansion resulting in high purity and high yield of RACR-NK cells.

As seen in FIG. 2B, immunophenotyping of IL-2 vs. RACR-expanded bdNK cells was performed to determine the activity of NK cells after RACR-expansion. bdNK cells were isolated from PBMCs, transduced and expanded in the presence of K562 feeder cells and either IL-2 or AP21967 using a similar protocol as described above. 39 days after transduction, CAR expression was analyzed by flow cytometry. CAR expression is shown on the top panels for NK cells expanded in the presence of either IL-2 (1000 U/ml) or AP21967 (20 nM). CD56 and CD16 expression on the CAR+ cells is shown on the bottom panels. RACR-Expanded NK cells showed a CD56 high phenotype similar to IL-2 expanded cells, with a slight decrease in the CD16 high population (81% vs. 96%) (FIG. 2B).

Comparing the functionality of IL-2 vs. RACR-expanded bdNK cells was determined by their ability to recognize and target tumor cells (FIG. 2C). NK cells naturally target tumors via innate stress ligands that are overexpressed on transformed cancer cells. Thus, innate- as well as CAR-mediated killing of tumor cells by CD19-CAR-RACR-expanded bdNK cells (AP-20) was measured. NK cells were isolated from PBMCs, transduced and expanded in the presence of K562 feeder cells and AP21967 (20 nM) using a similar protocol as described above with slight alterations in timing of AP21967 and K562 feeder cell addition (FIG. 2C-top panel experimental timeline). CAR expression is shown on the upper right panel at Day 31 after transduction. On Day 32, CAR+ NK cell function was assessed using intracellular cytokine production after tumor cell stimulation. 1×10⁵ NK cells were incubated with 1×10⁵ K562 cells, Nalm6 cells, or Nalm6 CD19 knock out cells in the presence of brefeldin A, monensin, and anti-CD107 for 5 hours in 100 μl RPMI media in a 96 well plate. CAR+ NK cells were gated on viable, CD3−, CD56+, and 2A+ cells. Shown are levels of CD107a (FIG. 2C bottom left panel) and IFNγ (FIG. 2C bottom middle panel) expression on AP21967-expanded CAR+ cells (AP-20) or untransduced controls. AP-20 cells released CD107a in response to innate receptor stimulation from highly reactive K562 tumor line cells similar to IL-2 expanded NK cell controls. Nalm6 cells induced lower CD107a secretion compared to K562 cells due to the lower level of innate stimulating ligands present. Nalm6 cells were then used to test the function of the CD19CAR targeting. Nalm6 cells expressing CD19 induced high levels of CD107a secretion from CD19-CAR-RACR-expanded bdNK cells (AP-20) compared to Nalm6 CD19 KO controls indicating robust CD19-CAR function in AP-20 NK cells. IFNγ release was also increased in response to CD19-Nalm6 cells compared to CD19 KO controls, further demonstrating CAR activity of AP-20 expanded NK cells.

Direct clearance of tumor cells via both innate receptor and CAR receptor targeting was measured. AP-20 CAR+ NK cell function was assessed using a flow cytometry-based killing assay. Target Nalm6 or Nalm6-CD19 knock out cells were labeled with Cell Trace Violet for 20 min at 37° C. at 0.5 mM in PBS and then washed. 5×10⁴ target cells were added to a 96 well plate and NK effector cells were added at varying Effector cell:Target cell (E:T) ratios in a total volume of 100 μl. After 5 hours 5×10⁴ counting beads were added to each well as a normalization factor. The cell and bead mixture was washed and viable, CD56−, Cell Trace Violet+ cells were analyzed. % Dead was calculated by comparing the total number of Viable, Cell trace violet+ cells in each well to a non-effector control well (FIG. 2C bottom right panel). AP-20 bdNK cells were able to recognize and kill Nalm6 cells via innate receptor targeting similar to IL-2 expanded untransduced bdNK cells showing no loss in cytotoxicity or function. Increased killing of CD19-expressing Nalm6 cells was observed with AP-20 cells compared to untransduced controls or CD19 knock out cells indicating synergistic increases in targeting of tumors via both innate and CAR-mediated killing.

### Example 2: Generation of iPSC-Derived CIL Cells

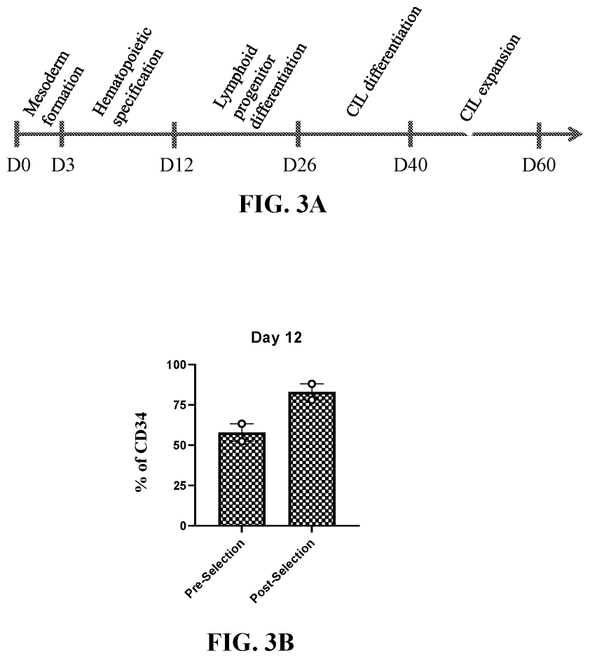

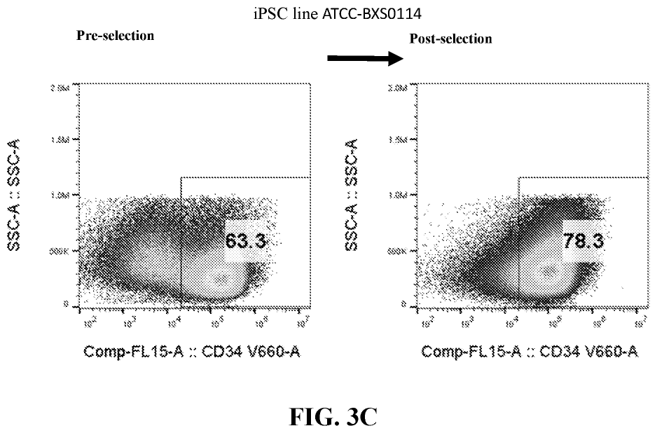

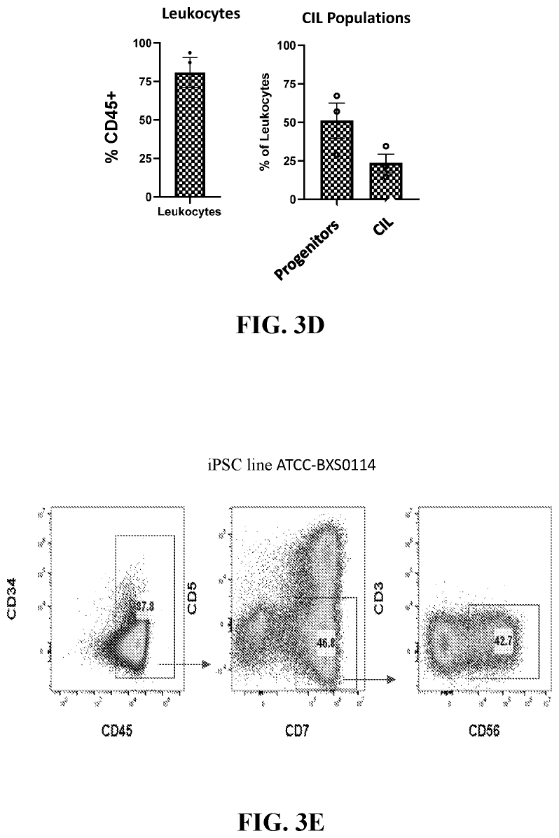

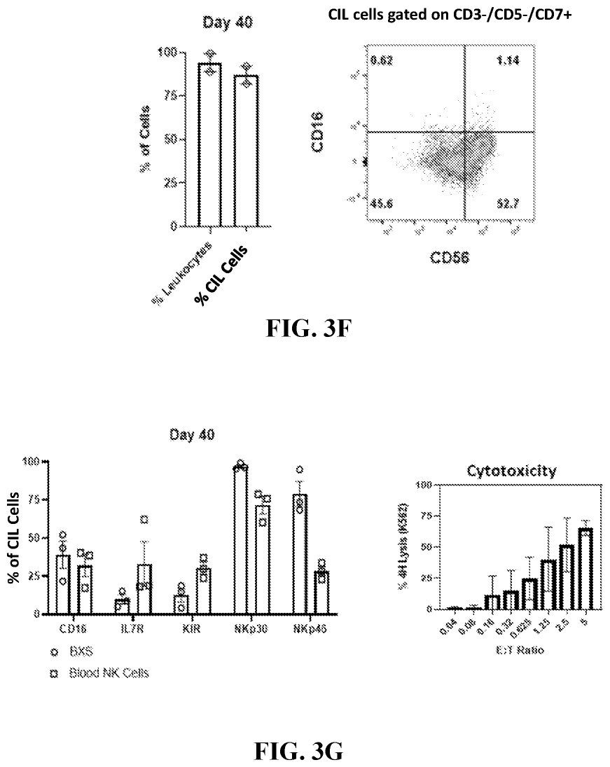

Undifferentiated human iPSCs were cultured in mTeSR Plus media (STEMCELL® Technologies) on hESC-qualified Matrigel (Corning®) and passaged using EDTA (Thermo Fisher Scientific®). iPSCs were differentiated into CIL cells using the STEMdiff NK Cell Kit (STEMCELL® Technologies) following the manufacturer's instructions. Briefly, at Day 0 a single-cell suspension of undifferentiated iPSCs was seeded into microwells (500 cells/microwell) to generate embryoid bodies (EBs) in STEMdiff Hematopoietic-EB Basal Medium supplemented with EB Supplement A (Days 0-3) and then EB Supplement B (Days 3-12). At Day 5, EBs were harvested from microwells and re-seeded onto non-tissue culture-treated plates. At Day 12, EBs were harvested and dissociated into a single-cell suspension, CD34+ cells selected using an EasySep Human CD34 Positive Selection Kit II (STEMCELL® Technologies) and analyzed using flow cytometry. An example selection (FIG. 3B) and flow cytometry analysis (FIG. 3C) are shown using a representative iPSC line, ATCC-BXS0114 (ACS-1028) (ATCC). The CD34+ selection process significantly enriched the cultures for CD34+ cells. Selected cells were re-seeded onto non-tissue culture-treated plates coated with StemSpan® Lymphoid Progenitor Differentiation Coating and cultured with StemSpan® SFEM II media supplemented with StemSpan® Lymphoid Progenitor Expansion Supplement (Days 12-26). Between Days 15-19, cells were harvested and re-seeded onto a newly coated plate. At Day 26, lymphoid progenitor cells were harvested and analyzed using flow cytometry (FIG. 3D and FIG. 3E). Cells at this timepoint were found to primarily be leukocytes (CD45+), and this population contained smaller populations of both progenitors (CD45+/CD5−/CD7+) as well as CIL cells (CD45+/CD5−/CD7+/CD56+). Cells were re-seeded onto tissue culture-treated, non-coated plates and cultured with StemSpan® SFEM II media supplemented with StemSpan® Cell Differentiation Supplement and UM729 (1 μM) (Days 26-40). At Day 40, CIL cells were harvested for further studies.

As seen in FIGS. 3F and 3G, at Day 40, iCIL cells were harvested and immunophenotyped by flow cytometry using the following antibodies: CD16 BUV496 (368), CD3 BUV661 (UCHT1), CD56 BV421 (HCD56), CD45 BV510 (2D1), CD5 BV605 (L17F12), NKG2D-BV785 (1D11), NKp30-BB700 (p30-15), CD7-PE (CD7-6B7), NKp46-PECy7 (9E2), CD127-AF700 (A019D5), KIR-APCFire750 (HP-MA4). iCIL cells were collected, pelleted, and resuspended in ZombieUV, diluted in PBS, and stained for 10 min. Cells were then resuspended in BD FACs buffer containing antibodies and stained for 20 min at 4° C. Cells were then processed on a Beckman Coulter CytoFLEX LX flow cytometer. Resulting FCS files were analyzed using BD FlowJo V10.8. The percentage of CD45+ cells are plotted as % Leukocytes and CD56+ cells are plotted as % CIL cells (FIG. 3F).

The resulting CIL cells displayed a CIL purity of >90% (FIG. 3G left panel, “BXS”). The CIL cells were largely CD56^{bright} with low CD16 expression (FIG. 3F right panel). Across many activation markers, it was found that the iCIL cells had a similar phenotype to blood derived NK cells (FIG. 3G left panel, “BXS”). The percentage of cells expressing CD16 was comparable between the iCIL and blood-derived NK populations (FIG. 3G left panel, “BXS”). However, blood-derived NK cells express higher amounts of IL-7RB, denoting a more immature and inhibitory phenotype, while Day 40 iCIL cells express higher amounts of activating receptors NKp30 and NKp46. These markers also suggest increased cytotoxic potential for the iCIL cells (FIG. 3G left panel, “BXS”).

The functionality of the iCILs was assessed through their ability to recognize and target tumor cells via recognition of innate stress ligands that are increased in transformed cells. iCIL cells were co-cultured with K562-mCherry target cells at different effector target ratios (E:T ratio) in cRPMI+IL-2 (RPMI+10% FBS+1×Glutamax+1×P/S+100 IU human IL-2) (FIG. 3G right panel). Cells were co-cultured for 4 hours at 37° C. followed by antibody-staining for CD56− BV421. % Lysis was calculated using Precision Counting Beads: #CD56− mCherry+ Target cells in CIL co-culture/#CD56− mCherry+ Target cells alone×100. iCIL cells were able to target and clear K562 tumor cells with high reproducibility across iCIL cell preparations showing robust functionality (FIG. 3G right panel).

Examples below further describe that iPSC-derived CIL cells (iCIL cells) described above were generated and then engineered using a synthetic cytokine receptor, an IL-2-type rapamycin-activated cell cytokine receptor (RACR2), to drive expansion of these cells with an exogenous cytokine. A diagram of the process can be found in FIG. 1A and FIG. 1B.

### Example 3: Differentiation of RACR-iCIL Cells from Lymphoid Progenitor and Precursor Cells

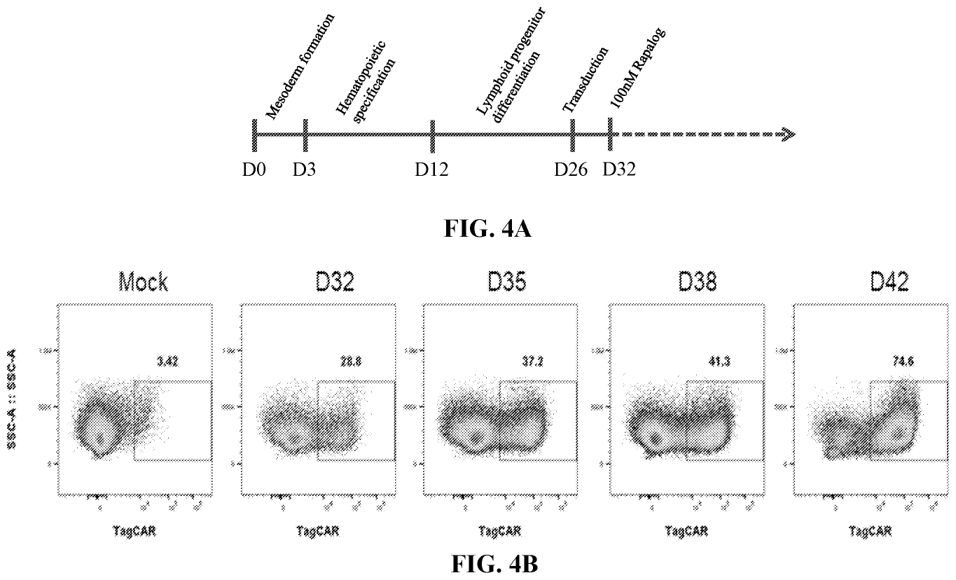

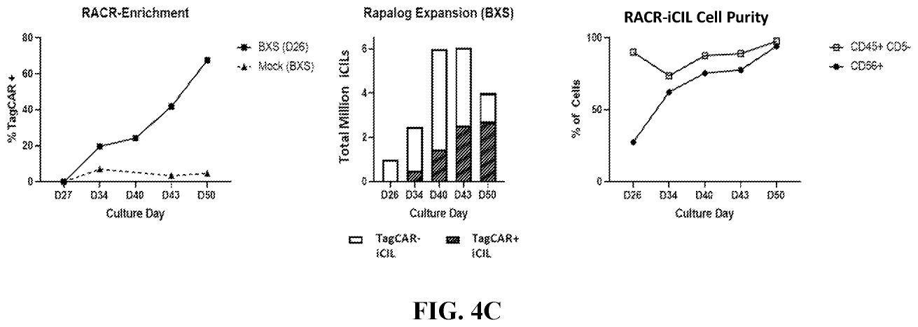

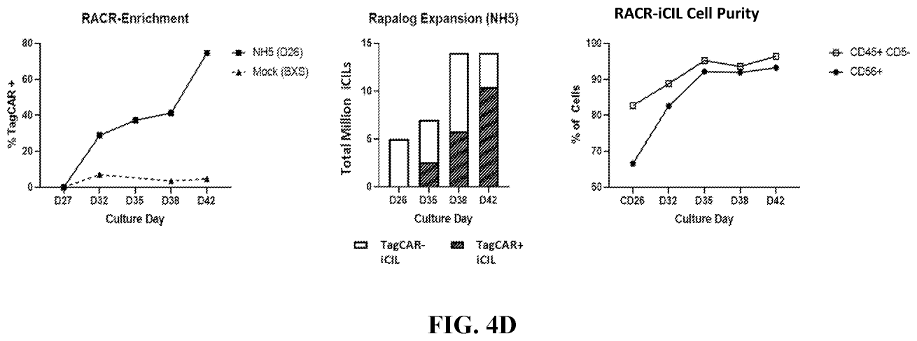

The RACR-system was utilized for differentiation to iPSC-derived CIL cells (iCIL cells). A timeline of the experiment is shown in FIG. 4A. The iPSC cell line ATCC-BXS0117 (ATCC #ACS-1031) was differentiated into lymphoid progenitors through the STEMdiff NK cell kit (STEMCELL® Technologies, #100-0170) as described in Example 2 above, up until the Day 26 timepoint. The percentage of cells containing common lymphoid progenitors (CD45+/CD5+/CD7+), CIL precursors (CD45+/CD5−/CD7+), and early CIL cells (CD45+/CD5−/CD7+/CD56+) is seen in FIG. 3D with ˜50% of the cells presenting as CIL precursors and ˜25% of total cells presenting as early CIL cells. In this study, Day 26 cells were pelleted and placed in a CIL expansion media at 2×10⁶ cells/mL containing (IL-2, IL-15, IL-21, IL-18, IL-7 in complete RPMI media: RPMI1640, 10% FBS, 1× Pen-Strep, and 1× Glutamax). Activation beads (#130-094-483) were added to cells at a 2:1 ratio and cells were placed in 37° C. incubator overnight to increase transduction of cells.

The cells were engineered with a polynucleotide encoding the RACR synthetic cytokine as described in Example 1, a Tag (anti-FITC) CAR (SEQ ID NO: 13), and an FRB domain for expression of FRB as a diffusible soluble protein in the engineered cell, in which the components were separated by a cleavable linker. The polynucleotide construct payload is designated Tag (anti-FITC) CAR-P2A-FRB-P2A-RACR. At Day 27, the cells were transduced with either VSV-G lentivirus with the payload of Tag (anti-FITC) CAR-P2A-FRB-P2A-RACR at an MOI 10 with 10 μg/mL of protamine sulfate or treated with 10 μg/mL or protamine sulfate alone (Mock). The next day a 50% media change was performed, and cells were given cRPMI1640 media supplemented with IL-2, IL-7, and IL-21. On Day 34, media was removed, and Mock cells were placed in cRPMI1640 media supplemented with CIL expansion media and RACR-transduced cells were then placed in cRPMI1640 media supplemented with AP21967 (A/C Heterodimerizer-Takara) at 100 nM to engage RACR-signaling. Subsequently, 50% media changes were performed weekly with 2× media. An aliquot of cells was collected weekly, counted on the Vicell Blu automated cell counter, and stained for CD16 clone 3G8, CD56 clone HCD56, CD45 clone 2D1. CD5 clone L17F12, CD7 clone CD7-6B7 and anti-FITC CAR (TagCAR) detection through FITC-AF647.

In response to rapalog treatment the RACR-iCIL cells demonstrated increased purity of TagCAR-RACR iCIL cells as indicated by increased % FITC-AF647 positive, expansion of TagCAR-RACR iCIL cells as indicated by the total number of TagCAR positive cells (FIG. 4B), as well as robust iCIL cell differentiation as indicated by increased percentage of CD56 positive cells over time as shown in FIG. 4C. This data demonstrates that rapalog AP21967 and the RACR system are sufficient to drive iCIL expansion in the absence of any other cytokine signaling. A similar process was followed for an additional iPSC line (NH5 NIH iPSC cell line) with very similar results as seen in FIG. 4D.

### Example 4: RACR-iCIL Cells Generated on Day 26 Display a Similar Phenotype and Cytotoxicity to Cytokine-Generated iCIL Cells

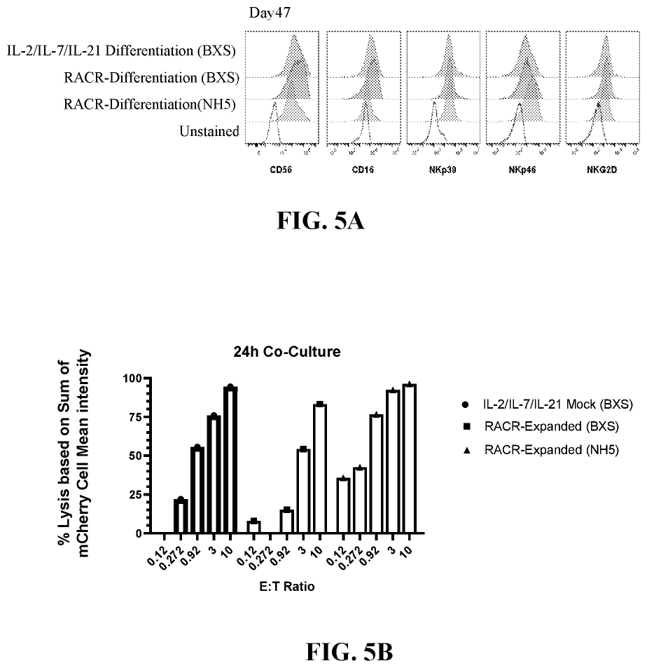

Functional assays were performed on RACR-expanded cells and stained cells for phenotypic markers CD7, CD5, CD45, CD56, NKG2D, NKp30, NKp46, and CD16 as shown in FIG. 5A. On Day 47. IL-2/IL-7/IL-21 Mock iCIL cells as well as RACR-expanded iCIL cells, generated as described in Example 3, were collected and pelleted and resuspended in ZombieUV diluted in PBS and stained for 10 min. Cells were then resuspended in BD FACs buffer containing antibodies and stained for 20 min at 4° C. Cells were then washed in BD FACs buffer 2× and analyzed on the Cytoflex flow cytometer.

RACR-expanded iCIL cells showed similar phenotype to cytokine expanded Mock cells with high expression of CD56, NKp30, NKp46, and NKG2D (FIG. 5A). iPSC line differences were observed with the NH5 cell line showing lower expression of CD16 than the BXS cells. This data demonstrates that RACR-iCIL cells have a highly activated phenotype with no decrease in activation receptors due to RACR-expansion as compared to cytokine expanded cells.

The cytotoxicity of RACR-expanded cells was tested against the MDA breast tumor cell line. Mock expanded or RACR-expanded iCIL cells were mixed with MDA-mCherry cells for 24H and then the plate was washed prior to imaging on an Opera Phenix® microscope (FIG. 5B). The total mCherry signal per well was calculated using the Opera Phenix® analysis tools. The graph is normalized to no-iCIL cell wells to generate a % of cells killed. RACR-expanded cells showed comparable toxicity to Mock cytokine expanded cells further demonstrating their highly activated phenotype and function (FIG. 5B).

### Example 5: Expansion of RACR-iCIL Cells

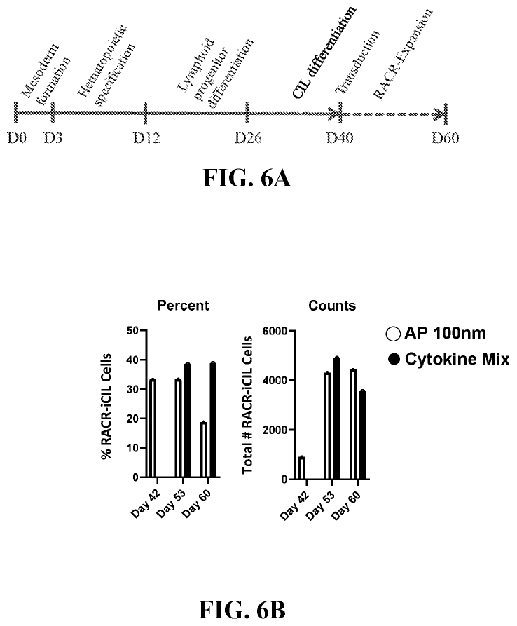

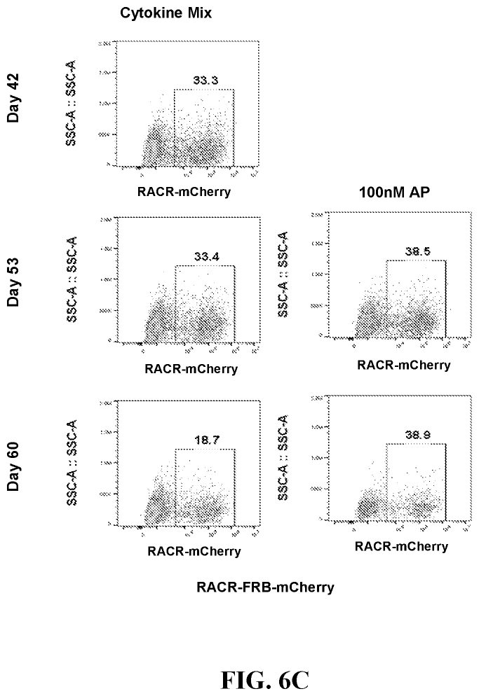

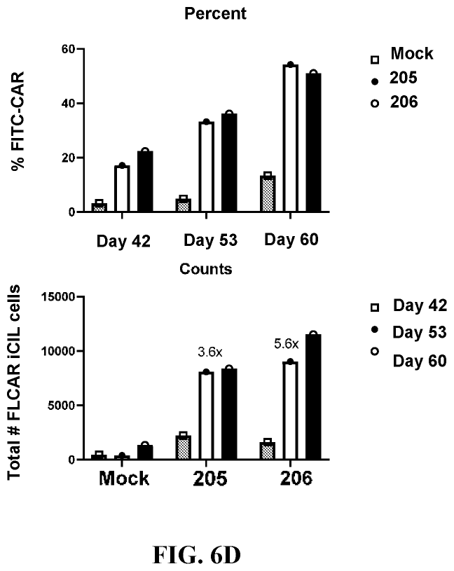

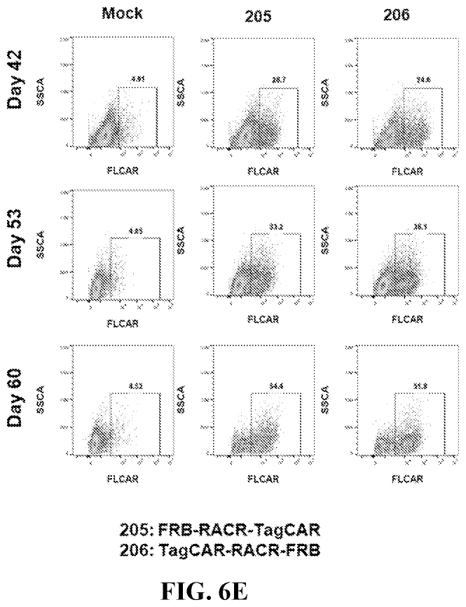

To investigate the ability of RACR to drive the expansion of iCIL cells, transduction and expansion of iCIL cells post differentiation at Day 40 was examined. iCIL cells were generated as described in Example 2. A timeline of the experiment is shown in FIG. 6A. In this experiments, the cells were engineered with a polynucleotide encoding the RACR synthetic cytokine as described in Example 1, an FRB domain for expression of FRB as a diffusible soluble protein in the engineered cell, and mCherry fluorescent protein. The polynucleotide construct payload is designated RACR-FRB-mCherry. In some experiments as described below, the TagCAR (e.g. SEQ ID NO: 13) replaced mCherry in the construct.

Activation beads (#130-094-483) were added to CIL cells at 2:1 ratio and cells were placed in 37° C. incubator overnight to increase transduction of cells. At Day 42, the cells were transduced with either VSV-G lentivirus with the payload of RACR-FRB-mCherry at an MOI 10 with 10 μg/mL of protamine sulfate or treated with 10 μg/mL of protamine sulfate alone (Mock) (FIG. 6B). The next day a 50% media change was performed, and cells were given CIL expansion media containing IL-2, IL-15, IL-21, IL-18, IL-7 in complete RPMI media: RPMI1640, 10% FBS, 1×Pen-Strep, and 1× Glutamax. On Day 45, media was removed, and Mock cells were placed in cRPMI1640 media supplemented with CIL expansion media and RACR-transduced cells were then placed in cRPMI1640 media supplemented with AP21967 (A/C Heterodimerizer-Takara®) at 100 nM to engage RACR-signaling. 50% media changes were performed weekly with 2× media, respectively. An aliquot of cells was collected weekly, counted on the Vicell Blu automated cell counter, and stained for CD56 clone HCD56 and RACR-FRB positive cells were detected through mCherry (FIG. 6C).

RACR-iCIL cells increased both their purity and yield through AP21967 expansion (FIG. 6B and FIG. 6C). The experiment was repeated with cells examined similarly as described above, however, different viral constructs containing TagCAR-RACR-FRB (206) and FRB-RACR-TagCAR (205) were observed (FIG. 6D and FIG. 6E).

In another experiment, previously isolated blood-derived NK cells were thawed and placed in RPMI media containing 10% FBS, Glutamax, Pen-strep, IL-2 (100 U/ml) on Day-1. On Day 0, the cells were washed in RPMI media and transduced with a CD19-RACR/VSVG lentiviral vector at an MOI of 20 in the presence of IL-2 (100 U/ml), and protamine sulfate (8 ug/ml). Three RACR constructs were transduced: RACR21 (IL-2RG/IL-21RB), RACR2 (IL-2RG/IL-2RB), and RACR7 (IL-2RG/IL-7RB). The RACR constructs were thus similar to the construct described in Example 1 encoding a beta chain from IL-2RB, except differing in the particular beta chain such that the IL-2RB of the RACR2 was replaced with an intracellular domain from either the IL-21RB (RACR21; e.g. SEQ ID NO:4) or the IL-7RB (RACR7; e.g. SEQ ID NO: 3)). Three days later, the media was replaced with fresh RPMI media containing IL-2 (100 U/ml). On Day 5, blood-derived NK cells were assessed for CD19 CAR expression by flow cytometry. On Day 7, cells were counted using the Vi-Cell® cell counter (Beckman) and divided into two equal groups. These two groups represented different expansion conditions for each RACR transduction construct. The two groups were 100 U/mL IL2 and Rapamycin. Next, after expansion additives were added to the cultures, the blood-derived NK cells were stimulated with irradiated (10,000 rads) K562 feeder cells expressing membrane-bound IL-21 and 41BBL at a ratio of ˜2 feeder cells:1 NK cell. On Day 10, the transduced cells received a half volume media change and were resuspended in fresh RPMI media containing either 2×IL-2 (100 U/ml) or 2× Rapamyacin. Beginning on Day 10, cells were stimulated with irradiated feeder cells weekly a half volume media change was performed as needed.

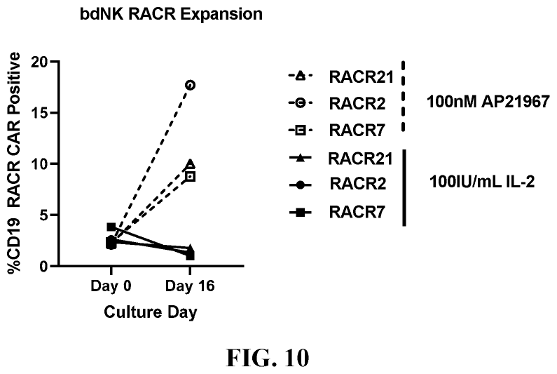

Cell counting and RACR-CAR expression analysis was performed a weekly basis. Cells were stained with a mix including: CD19 FMC63 (FITC), IL7Ra (APC700), CD56 (BV421), CD25 (V660), CD16 (NUV525), and Zombie UV (NUV450). As seen in FIG. 10, RACR2 treated with Rapamycin supported the highest RACR-expansion and both RACR7 and RACR21 treated with Rapamycin supported RACR expansion to a lesser extent. However, all three cell cultures transduced with RACR constructs and treated with Rapamycin showed significantly higher expansion in comparison to the three cell cultures transduced with RACR constructs and treated with IL-2 (FIG. 10).

### Example 6: Function of RACR-iCIL Cells

To investigate the functionality of Day 40 expanded RACR-iCIL cells in comparison to iCIL cells expanded in IL-2, immunophenotyping was performed as well as a post-expansion CD107a activity assay. Day 40 iCIL cells were generated with the same process as shown in Example 5 and these cells were transduced with TagCAR-RACR-FRB (206). RACR-iCIL cells were expanded in 100 nM AP21967 and Mock iCIL cells were expanded in 100 IU/mL huIL-2 until Day 60 and then harvested for functional assays. Cells were stained for CD16, CD56, NKp46, NKp30, NKG2A, NKG2D, CD3, CD5 and CD57 and flow cytometry staining was performed as described above.

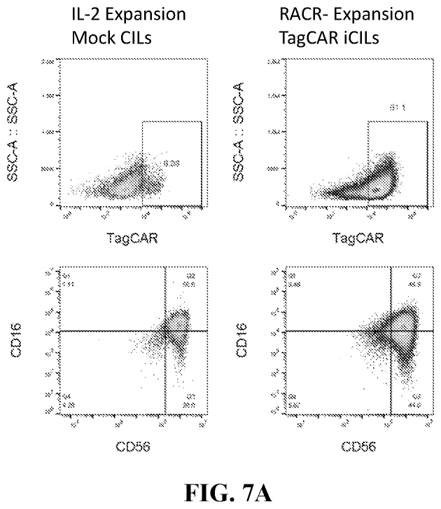

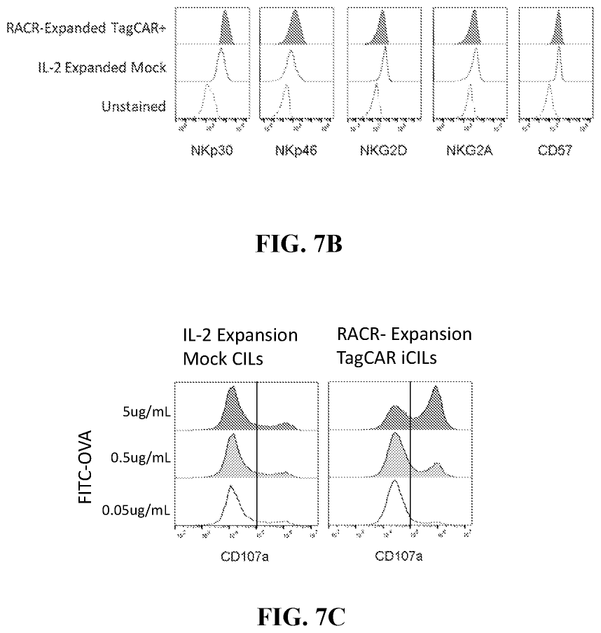

Day 60 RACR-expanded iCIL cells expressed high levels of TagCAR and these cells displayed similar phenotype to IL-2 expanded Mock cells with high expression of CD56 and CD16 (FIG. 7A). RACR-expanded TagCAR iCIL cells expressed higher levels of NKp46 and NKp30 than IL-2 expanded Mock iCIL cells, suggesting a more cytotoxic phenotype (FIG. 7B). Expression of NKG2A, NKG2D, and CD57 were unchanged between RACR-iCIL cells and IL-2 expanded control cells.

The ability of RACR-TagCAR expressing cells to secrete CD107a in response to antigen exposure was assessed. TagCAR antigen was coated on 96 plates in PBS at 5, 0.5, and 0.05 μg/mL for 1H at 37° C. Plates were washed and RACR-iCIL cells and IL-2 Mock iCIL cells were then added to the well in the presence of anti-CD107a PE in cRPMI media. Cells were incubated for 4 hours with 1× monesin followed by flow cytometry staining for CD56. Cells were pelleted, washed in BD FACs wash and then collected on Cytoflex flow cytometer. RACR-expanded TagCAR iCIL cells showed robust CD107a secretion in response to antigen indicating high cytotoxicity of RACR-expanded cells (FIG. 7C).

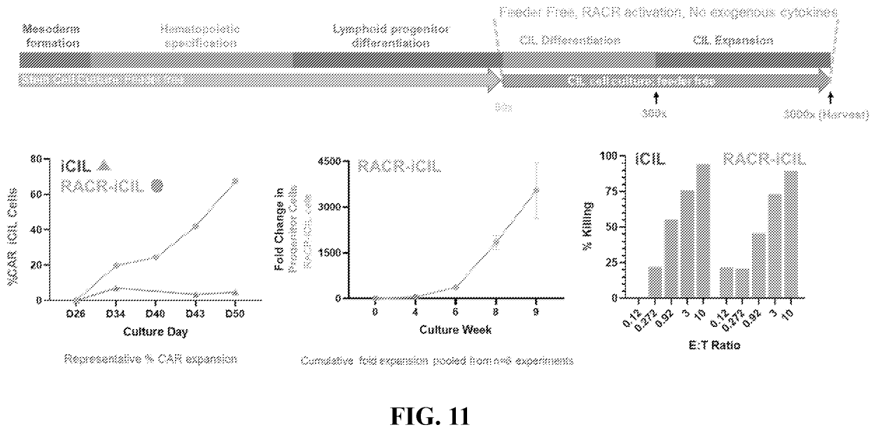

These data show that over the course of iCIL differentiation and feeder-free expansion, approximately 3000-fold increase of iCILs was observed (FIG. 11). Because the synthetic receptor induces a signal akin to IL-2 or IL-15, the iCILs possess a selective growth advantage, resulting in a highly pure iCIL population (FIG. 3G, left panel; FIG. 4B, and FIG. 7A). iCILs exhibit multifunctional properties, including cytotoxic activity against tumor target cells (FIGS. 3G right panel and 11). The iCILs, induced to expand with rapalog AP21967, also exhibited secretion of IFNγ and TNF-α, mediated by the synergistic activity of innate activating receptors and chimeric antigen receptor targeting (FIG. 7B).

### Example 7: RACR-Signaling to Replace Common Gamma Chain Cytokines

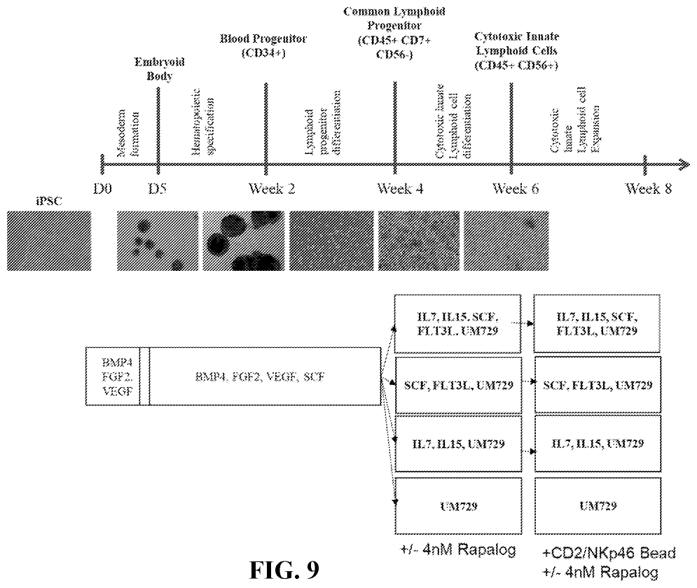

To investigate the ability of RACR-signaling to replace common gamma chain cytokines during differentiation from CLP into CIL cells, CLP cells were transduced at Day 26, prior to exposure to differentiation media. The transition from CLP to CIL generally requires the common gamma chain cytokines IL-7 and IL-15 with differentiation factors SCF, FLT3L, and UM729. A timeline of the experiment is shown in FIG. 9.

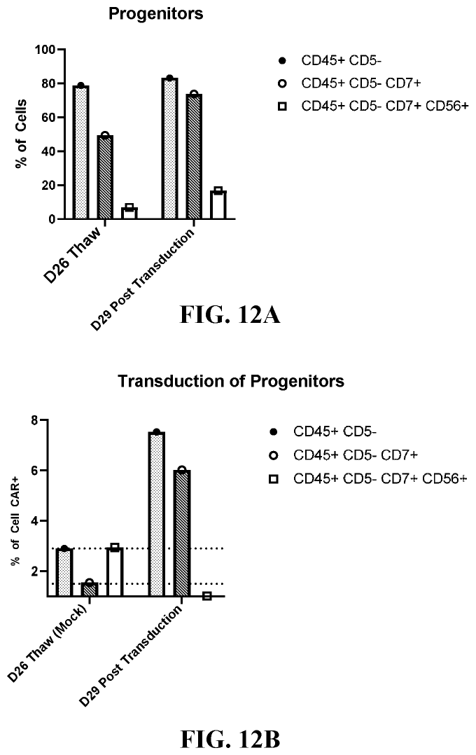

The iPSC line WTC11 was differentiated into lymphoid progenitors through the STEMdiff® cell kit (STEMCELL Technologies, #100-0170) as described above up until the Day 26 timepoint. The percent containing common lymphoid progenitors (CD45+/CD7+) was 49.4% in this preparation, with very few cells (approximately 10%) expressing CD56 (FIG. 12A). A 50% media change was performed using StemSpan® SFEM II media supplemented with StemSpan® Lymphoid Progenitor Expansion Supplement. The Day 26 cells were then transduced with VSV-G lentivirus with the payload of TagCAR-P2A-FRB-P2A-RACR, described in Example 3, at an MOI 10 and treated with 10 ug/mL of protamine sulfate or treated with 10 ug/mL of protamine sulfate alone without transduction (mock).

On Day 29, transduction of progenitors was measured and found that approximately 8% of CD45+ cells contained the construct (FIG. 12B). No transduction was detected in the CD56+ cell population (FIG. 12B).

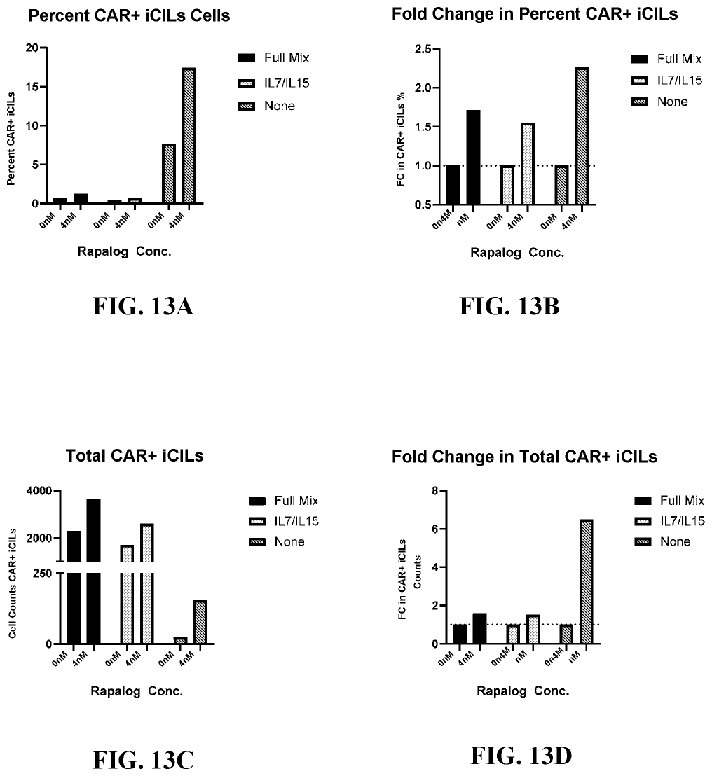

CD45+ cells were collected and reseeded into four conditions to induce differentiation from the transduced CLP to a CIL cell. Four culture conditions were examined: 1) StemSpan@ SFEM II media supplemented SCF, FLT3L, IL7, IL15, and UM729 (Full Mix); 2) StemSpan® SFEM II media supplemented SCF, FLT3L, and UM729 (SCF/FLT3L); 3) StemSpan® SFEM II media supplemented IL7, IL15, and UM729 (IL7/IL15); and 4) StemSpan® SFEM II media with UM729 alone (None). Cells were then further split into two treatment populations: with 4 nM AP21967 (rapalog) or without (0 nM rapalog). Cells were monitored for expression of the CAR with results shown in FIGS. 13A-13D. The largest fold change in RACR-CAR+ cells was observed in the condition with AP21697 alone (None), demonstrating the cell's dependence on the RACR-signal for cell growth (FIGS. 13B and 13D).

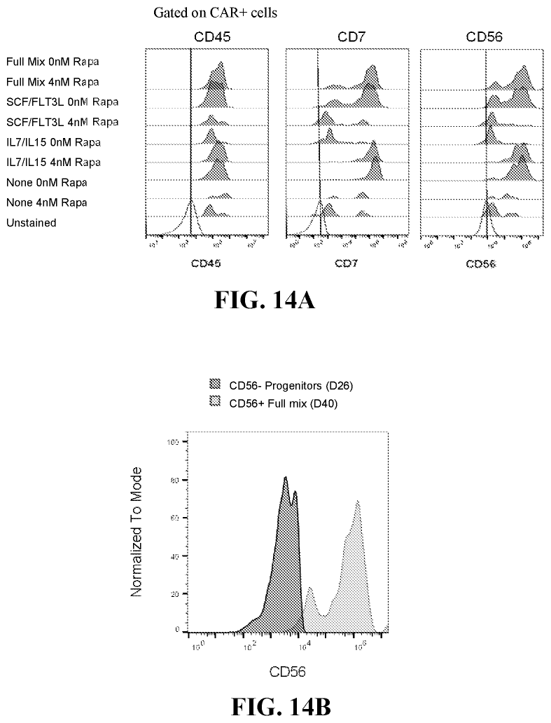

On Day 33, 50% well-volume of the same media was added and then a 50% media change was performed on Day 34. On Day 40, an aliquot of cells was collected and stained with CD16 clone 3G8, CD56 clone HCD56, CD45 clone 2D1, CD5 clone L17F12, CD7 clone CD7-6B7 and TagCAR detection through FITC-AF647 to analyze CIL cell differentiation and TagCAR expression (FIG. 14A). An increase in the percent of RACR-CAR+ CD56+ cells in response to AP21967 (rapalog) was observed in all conditions with the highest percent increase observed in conditions lacking IL7/IL15, because in those conditions the RACR provided the common gamma chain signal and RACR-CAR+ cell expansion (FIG. 14A). All RACR-CAR+ cells were observed to be CD45+. Lower levels of CD7 and CD56 were observed in cells grown in media not containing cytokines as compared to cytokine exposed cells (FIG. 14A). Progenitor cells analyzed on Day 26 displayed lower levels of CD56 as compared to cells exposed to the Full Mix analyzed on Day 40 (FIG. 14B). This data demonstrates that rapalog AP21967 and the RACR system may replace common gamma chain cytokines IL-7 and IL-15 during iCIL differentiation from the common lymphoid progenitors.

The synthetic cytokine receptor system may be employed ex vivo to generate immune effector cells in a manufacturing setting and has the potential to enable selective expansion and survival of the engineered cells in vivo. These data demonstrate the capacity of the disclosed methods and associated compositions to produce innate immune effector cells with significant potential as cancer therapies. This approach has the potential to improve manufacturing process control and product consistency, as well as decrease complexity of iPSC-based allogeneic cell therapy manufacturing. By rationally designing and deploying different types of synthetic receptors, the disclosed methods and associated compositions will be amenable to the production of immune effector cells for therapeutic application in hematological and solid tumor settings.

### Example 8: In Vivo Anti-Tumor Activity

The engineered CIL cells of the disclosure will be investigated in a NSG MHC knockout mice to evaluate the iCIL cell in vivo anti-tumor activity. This humanized mouse model is an immunodeficient NOD scid gamma mouse, which carries the strain NOD.Cg-Prkde^{scid} Il2rg^{tm1WjI}/SzJ. To establish a xenograft tumor model, tumors will be injected intraperitoneally or subcutaneously into the mice. RACR-iCILs will subsequently be injected either intraperitoneally or intravenously. To analyze the effect of RACR-iCILs on the xenograft tumors, tumor size will be measured by bioluminescence imaging (BLI) over time.

### Example 9: Generation of RACR and CAR-Engineered NKs and Assessment of Cytotoxic Killing Activity

An NK cell line (NK92) was transduced with lentiviral vectors containing free-FKBP12-rapamycin binding (FRB) as well as RACR (SEQ ID NO:32) and anti-FITC CAR (TagCAR). The nucleotide sequence encoding the TagCAR is set forth in SEQ ID NO: 13 and encodes the amino acid sequence set forth in SEQ ID NO:14; in which amino acids of the CD8a signal peptide MALPVTALLLPLALLLHAARP (SEQ ID NO: 17) are removed when expressed by the cell (see also e.g. published PCT Appl. No. WO2019144095).

Cells were expanded in 100 nM rapamycin or 100 IU/mL of IL-2 for months to enrich for RACR+ cells. RACR-NK cells were then incubated with different solid human tumor cells expressing a nuclear fluorescent protein at a 10:1 T:E ratio in the presence of media only (unstimulated NK), media supplemented with IL-2 and IL-15 (cytokine-stimulated NK), or supplemented with 100 nM Rapamycin (RACR-stimulated NK).

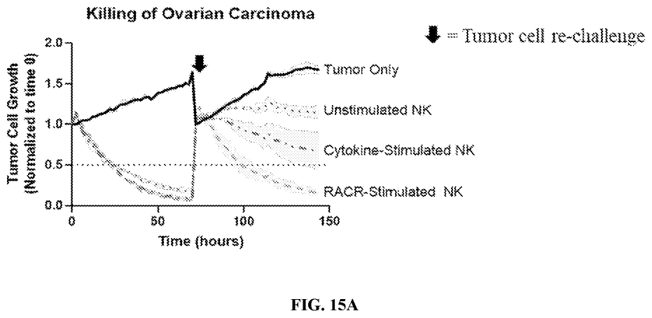

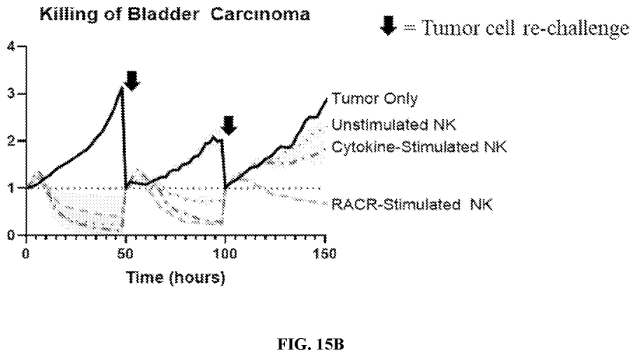

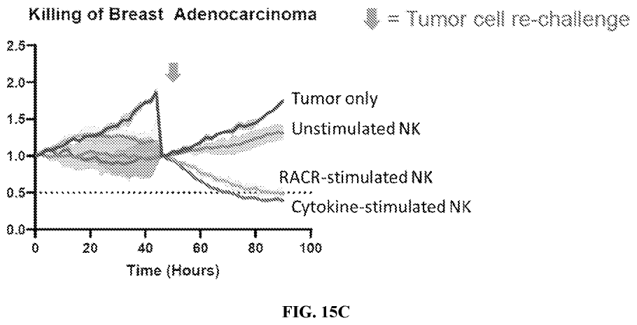

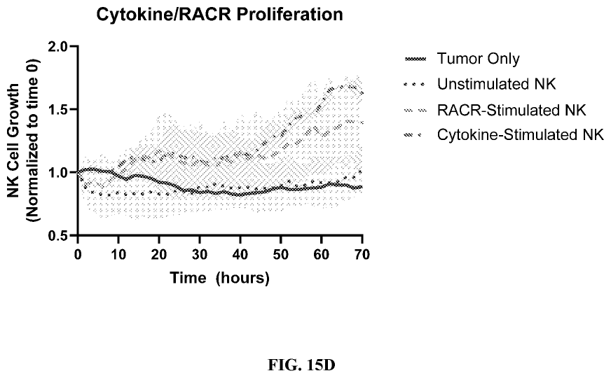

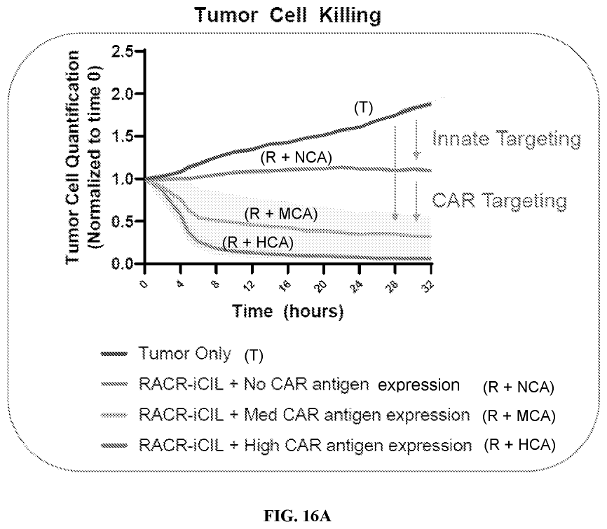

RACR-NK cells and tumor cells were placed into an IncuCyte fluorescent microscope and imaged every 2 hours during co-culture for up to 150 hours. The number of tumor cells was quantified over time via a fluorescent marker and graphed as the ratio of tumor cells compared to time 0 (time 0 is equal to one). At about 40 hours, 50 hours or 100 hours of co-culture, the RACR-NK cells were collected, placed in fresh media and then re-exposed to the tumor cells for a rechallenge. The RACR-NK cells exhibited functional killing of different solid tumor lines including bladder, Heme, colon, breast, ovarian an uterine solid tumor lines. Exemplary results for cytotoxic killing are shown for an ovarian carcinoma (FIG. 15A), a bladder carcinoma cell line (FIG. 15B) and an breast adenocarcinoma cell line (FIG. 15C). The extent of tumor-cell killing, particularly after rechallenge, was substantially greater for the RACR-stimulated NK cells than the cytokine-stimulated NK cells or unstimulated NK cells. Specifically, at first exposure to the tumor all RACR-NK cells cleared regardless of stimulation; however, upon repeated tumor challenge RACR-stimulation via rapamycin supported much higher levels of killing.

NK cell proliferation was also monitored over time during the co-culture. Tracking the expansion of NK cells during tumor-killing of the bladder carcinoma cell line showed that the RACR-stimulated NK cells exhibited the greatest proliferation (FIG. 15D), which may contribute to the increase in the ability of the RACR-stimulated NK cells to serially kill the tumor cells.

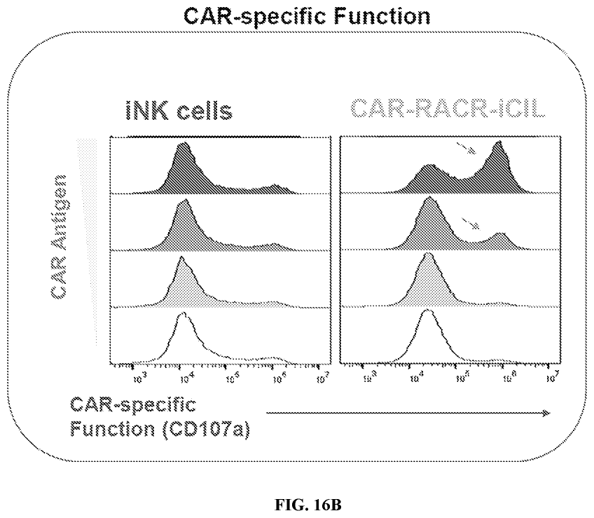

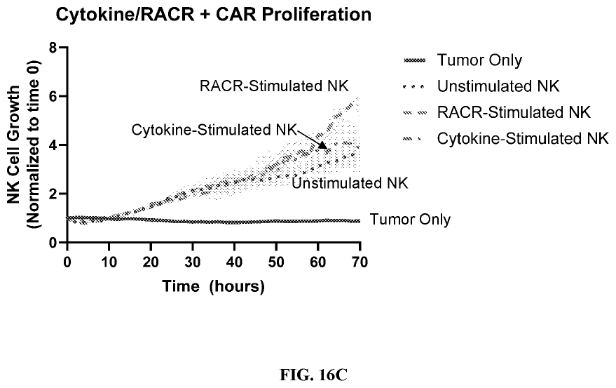

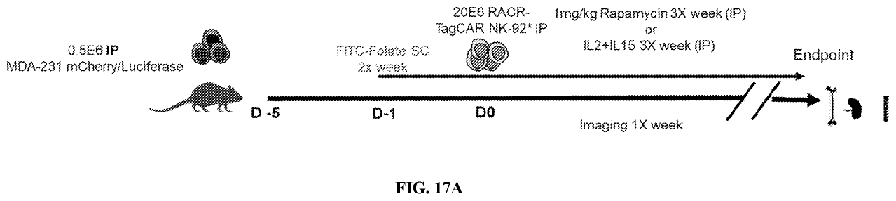

To assess the activity of the CAR expressed by the RACR-NK cells, target cells, a tumor killing assay was performed. As shown in FIG. 16A, RACR-NK cells stimulated with rapamycin exhibited tumor cell killing even in the absence of CAR antigen (“No CAR antigen”). Killing activity was substantially increased in the presence of the Folate-Fluorescien bound to folate receptor expressed on the tumor cells, and the level of activity correlated with antigen expression (medium, “Med CAR antigen expression”; or high, “High CAR antigen expression.) (FIG. 16A).

As an alternative to cell killing, functional activity of NK cells also can be monitored by the marker CD107a, which is a degranulation marker of NK cell functional activity. The CAR antigen-expressing target cells were then cultured with unstimulated NK cells or CAR-RACR-NK cells. As shown in FIG. 16B, the extent of CD107a directly correlated with the level of CAR antigen, indicating a CAR-specific functional activity of the CAR-RACR-NK cells.

NK cell proliferation was also monitored over time during the co-culture. Tracking the expansion of NK cells during tumor-killing of the target cells showed that CAR targeting further increases RACR-NK proliferation, suggesting that RACR and CAR activation can work synergistically to increase RACR-NK proliferation and CAR-mediated targeting (FIG. 16C).

### Example 10: CAR-RACR-iCILS Display Potent and Specific CAR-Driven and Innate Tumor Killing

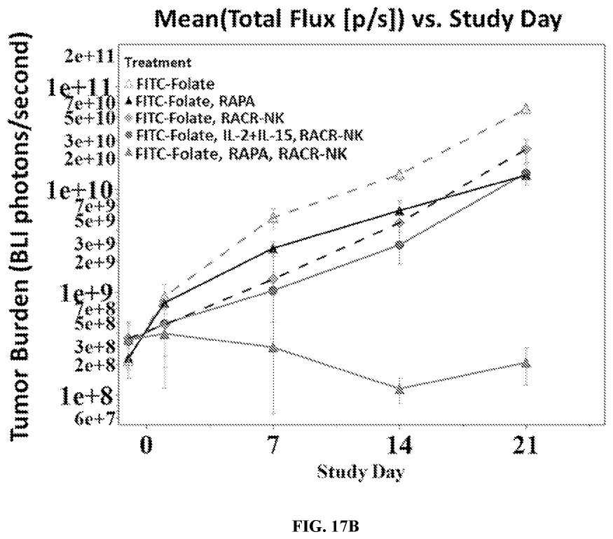

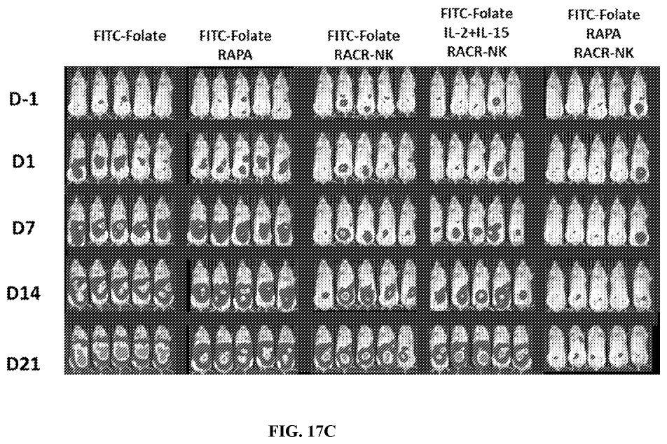

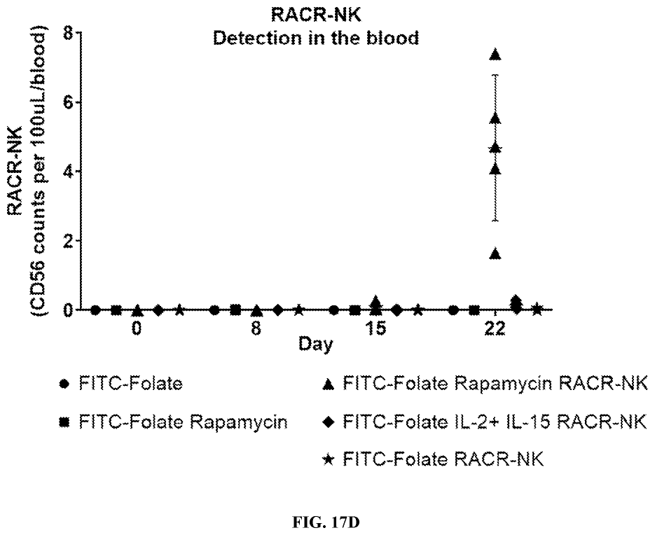

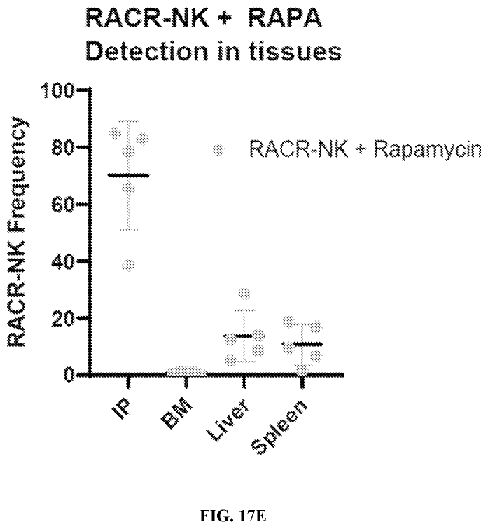

RACR-TagCAR-NK cells, generated as described in Example 9, were tested for their ability to respond to RACR stimulation and control tumors in vivo in a mouse model (FIG. 17A). On Day 5, 5×10⁶ MDA-MB231 mCherry-FFLUC breast cancer cells were injected into the intraperitoneal region of NSG-mice. On Day −1, mice were injected with 100 nM Folate-Fluorescein (Folate-FITC; injected SC two times per week) to tag the tumor cells via the folate receptor. Mice were randomized for tumor size and on Day 0, 20×10⁶ RACR-TagCAR-NK cells were injected into the intraperitoneal region and mice were either given no stimulation, huIL-2+huIL-15 (injected IP three times per week), or rapamycin (injected IP three times per week). The tumor was imaged via bioluminescence imaging (BLI) weekly to monitor tumor progression.

All control mice, other than those treated with RACR-TagCAR-NK cells, had rapid tumor growth as shown by BLI imaging regardless of rapamycin dosing (quantification of luminescence shown in FIG. 17B, and raw images shown in FIG. 17C). There was a reduction in tumor growth in groups dosed with RACR-TagCAR-NK cells and increased RACR-NK cells detected in the blood and peritoneum of mice (FIGS. 17D and 17E), which shows the ability of NK cells to target the tumor. However, by day 7 post-RACR-TagCAR-NK injection the rapamycin-treated group began to show improved tumor control compared to the unstimulated or cytokine-stimulated groups. This data demonstrates the superior ability of RACR-stimulation to support tumor killing in vivo, compared even to the “best in class” of cytokine stimulation.

### Example 11: RACR-iCILs Demonstrate Early and Potent Tumor Killing

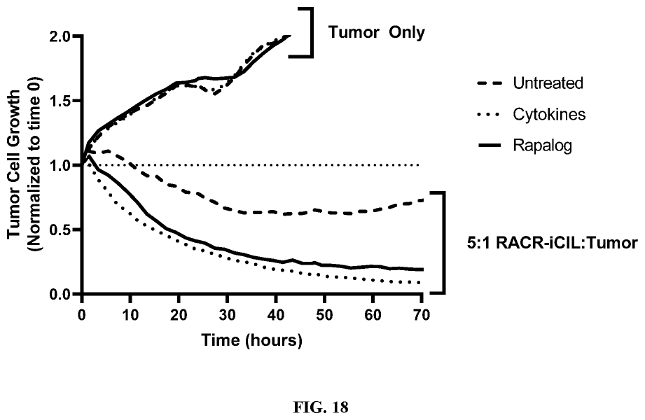

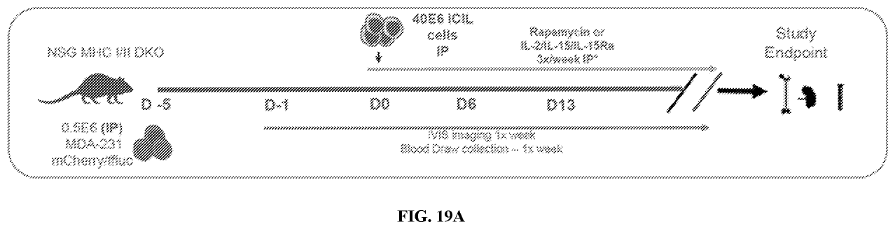

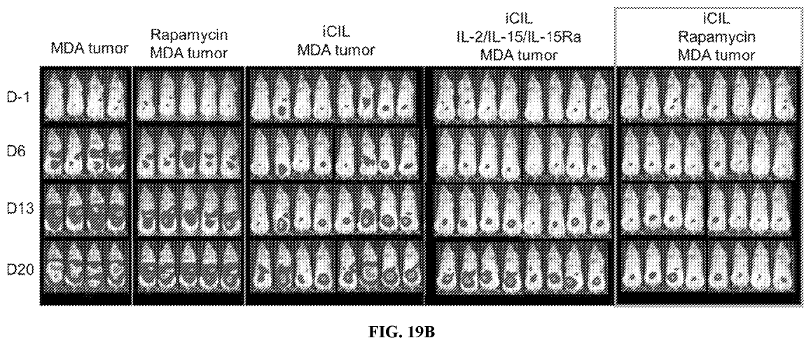

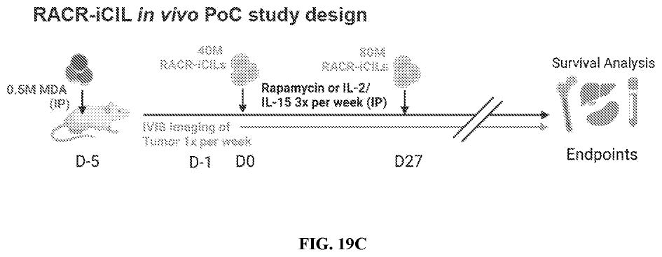

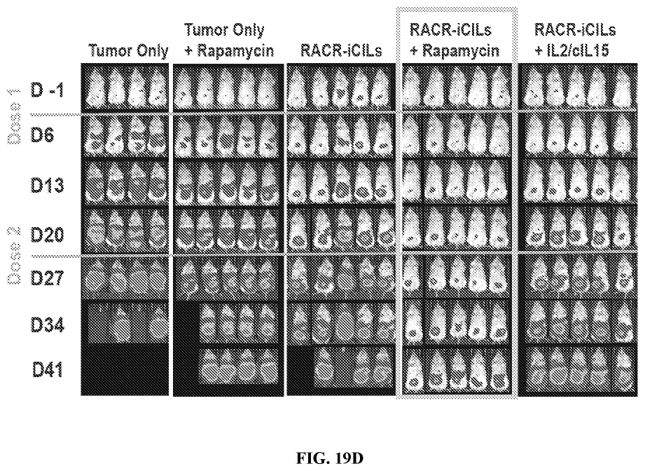

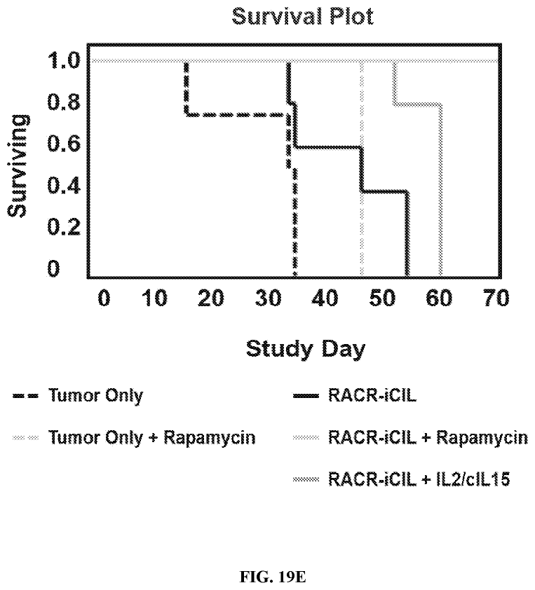

RACR-iCIL tumor killing ability was determined in vitro (FIG. 18) and in vivo (FIGS. 19A and 19B) using the RACR-iCILs generated in Example 9. In vitro tumor killing was determined by co-culturing RACR-iCILs with the breast adenocarcinoma cell line MDA-MB-231. RACR-iCILs were co-cultured with MDA-MB-231 cells at a ratio of 5:1 and either left untreated, treated with cytokines, or treated with rapalog. Rapalog and cytokine activation of RACR-iCILs enhanced tumor killing compared to untreated RACR-iCILs (FIG. 18).

In vivo tumor killing was determined in a mouse model. A timeline of the experiment is shown in (FIG. 19A). On Day −5, 5×10⁶ MDA-MB-231 mCherry-FFLUC breast cancer cells were injected into the intraperitoneal region of NSG MHC I/II DKO mice.

Beginning on Day −1, mice were imaged once per week and their blood was collected once per week until the end of the experiment. Mice were randomized for tumor size and on Day 0, 40×10⁶ RACR-iCIL cells were injected into the intraperitoneal region and mice were either given no stimulation, IL-2/IL-15/IL-15Ra (injected IP three times per week), or rapamycin (injected IP three times per week). Control mice received either MDA-MB-231 cells alone, MDA-MB-231 cells and rapamycin, or MDA-MB-231 cells and iCIL. The tumor was imaged via bioluminescence imaging (BLI) weekly to monitor tumor progression.

All control mice, other than those treated with RACR-iCIL cells, had rapid tumor growth by Day 6 as shown by BLI imaging (first and second panel from the left in FIG. 19B). There was a reduction in tumor growth in mice administered RACR-iCILs only (third panel in FIG. 19B), which was further reduced at Day 6 with administration of IL-2/IL-15/IL-15Ra or rapamycin (fourth and fifth panels in FIG. 19B). However, over 20 days, the greatest reduction in tumor growth was achieved with administration of rapamycin. This data demonstrates the superior ability of RACR-iCIL stimulation to support early tumor killing in vivo. This data also demonstrates the superior ability of RACR-iCIL stimulation to support tumor killing in vivo compared even to the “best in class” of cytokine stimulation.

In a separate experiment, in vivo tumor killing was determined in a mouse model where mice received two separate doses of RACR-iCILs on the first day of the experiment and day 27 (FIG. 19C). All control mice, other than those treated with RACR-iCIL cells, had rapid tumor growth by Day 6 as shown by BLI imaging (first and second panel from the left in FIG. 19D). There was a reduction in tumor growth in mice administered RACR-iCILs only on Day 6 (third panel in FIG. 19D), which was further reduced at Day 6 with administration of IL-2/IL-15/IL-15Ra or rapamycin (fourth and fifth panels in FIG. 19D). However, over 41 days, the greatest reduction in tumor growth was achieved with administration of rapamycin. Furthermore, mice receiving RACR-iCIL and rapamycin had 100% survival on Day 70 (FIG. 19E). This data demonstrates the superior ability of RACR-iCIL stimulation to support early tumor killing in vivo. This data also demonstrates the superior ability of RACR-iCIL stimulation to support tumor killing in vivo compared even to the “best in class” of cytokine stimulation.

### Example 12: RACR-Expansion of Blood-Derived Vδ2+ T Cells

This example demonstrates the ability of the RACR system to drive expansion of Vδ2+ T cells. On day 0, peripheral blood monocular cells (PBMCs) were cultured in a 37° C. incubator with 5 μM Zometa and IL-2 (500 IU/mL) in complete RPMI (cRPMI) media containing RPMI1640, 10% FBS, 1× Pen-Strep, and 1× Glutamax for initial in vitro expansion. On day 5, the cells were washed in cRPMI and transduced with lentivirus containing RACR-FRB-mCherry (described in Example 5; at an MOI of 10 in the presence of IL-2 (500 IU/mL). Cells were split in cRPMI media containing 500 IU/mL IL-2 every 2-3 days. An aliquot was collected and initial transduction of cells was assessed by mCherry via flow cytometry on day 9.

Vδ2+ T cells were then cultured in wells containing fresh cRPMI with either IL-2 (500 IU/mL), Rapamycin (10 nM), or AP21967 (A/C Heterodimerizer-Takara®, 10 nM) to initiate RACR signaling for an additional 5 days. On day 14, an aliquot of the cells was collected and washed, stained for V32, and RACR transduction was measured by mCherry. Percent Vδ2+ RACR+ cells, as well as number of transduced V32+ cells measured by counting beads was assessed by detection of V82 stain and mCherry via flow cytometry. Similarly, on day 21, an aliquot of cells was again measured for percent and number of Vδ2+ RACR+ cells via flow cytometry.

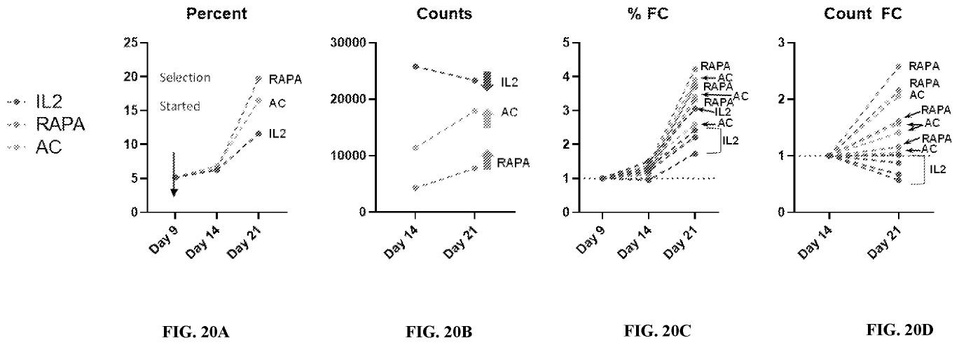

Vδ2+ RACR+ cells increased in both percentage and total number of cells on days 14 and 21 when cultured with either of Rapamycin or AP21967 for cells transduced with RACR (FIG. 20A-D). In contrast, cell cultures transduced with RACR and treated with IL-2 resulted in a lower total number of transduced Vδ2+ cells on day 21 as compared to day 14 (FIG. 20A-D).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

TABLE OF SEQUENCES

| SEQ ID NO: | Sequence | Description |
|---|---|---|
| 1 | ERTMPRIPTLKNLEDLVTEYHGNFSAWSGVSKGLAE | IL2RG |
| | SLQPDYSERLCLVSEIPPKGGALG | Common |
| | EGPGASPCNQHSPYWAPPCYTLKPET | Gamma |
| | | Chain |
| | | Intracellular |
| | | domain |
| 2 | NCRNTGPWLKKVLKCNTPDPSKFFSQLSSEHGGDVQ | IL2RB |
| | KWLSSPFPSSSFSPGGLAPEISPL | intracellular |
| | EVLERDKVTQLLLQQDKVPEPASLSSNHSLTSCFTN | domain |
| | QGYFFFHLPDALEIEACQVYFTYD | |
| | PYSEEDPDEGVAGAPTGSSPQPLQPLSGEDDAYCTFP | |
| | SRDDLLLFSPSLLGGPSPPSTAP | |
| | GGSGAGEERMPPSLQERVPRDWDPQPLGPPTPGVPD | |
| | LVDFQPPPELVLREAGEEVPDAGP | |
| | REGVSFPWSRPPGQGEFRALNARLPLNTDAYLSLQE | |
| | LQGQDPTHLV | |
| 3 | KKRIKPIVWPSLPDHKKTLEHLCKKPRKNLNVSFNPE | IL7RB |
| | SFLDCQIHRVDDIQARDEVEGFL | intracellular |
| | QDTFPQQLEESEKQRLGGDVQSPNCPSEDVVITPESF | domain |
| | GRDSSLTCLAGNVSACDAPILSS | |
| | SRSLDCRESGKNGPHVYQDLLLSLGTTNSTLPPPFSL | |
| | QSGILTLNPVAQGQPILTSLGSN | |
| | QEEAYVTMSSFYQNQ | |
| 4 | SLKTHPLWRLWKKIWAVPSPERFFMPLYKGCSGDF | IL21RB |
| | KKWVGAPFTGSSLELGPWSPEVPST | intracellular |
| | LEVYSCHPPRSPAKRLQLTELQEPAELVESDGVPKPS | domain |
| | FWPTAQNSGGSAYSEERDRPYGL | |
| | VSIDTVTVLDAEGPCTWPCSCEDDGYPALDLDAGLE | |
| | PSPGLEDPLLDAGTTVLSCGCVSA | |
| | GSPGLGGPLGSLLDRLKPPLADGEDWAGGLPWGGR | |
| | SPGGVSESEAGSPLAGLDMDTFDSG | |
| | FVGSDCSSPVECDFTSPGDEGPPRSYLRQWVVIPPPL | |
| | SSPGPQAS | |
| 5 | GVQVETISPGDGRTFPKRGQTCVVHYTGMLEDGKK | FKBP |
| | FDSSRDRNKPFKFMLGKQEVIRGWE | domain |
| | EGVAQMSVGQRAKLTISPDYAYGATGHPGIIPPHAT | |
| | LVFDVELLKLE | |
| 6 | ILWHEMWHEGLEEASRLYFGERNVKGMFEVLEPLH | FRB domain |
| | AMMERGPQTLKETSFNQAYGRDLME | |
| | AQEWCRKYMKSGNVKDLTQAWDLYYHVFRRISK | |
| 7 | ILWHEMWHEGLEEASRLYFGERNVKGMFEVLEPLH | variant FRB |
| | AMMERGPQTLKETSFNQAYGRDLME | domain (FRB |
| | AQEWCRKYMKSGNVKDLLQAWDLYYHVFRRISK | mutant |
| | | domain) |
| 8 | VVISVGSMGLIISLLCVYFWL | transmembrane |
| | | (TM) |
| | | domain |
| 9 | VAVAGCVFLLISVLLLSGL | TM domain |
| 10 | PILLTISILSFFSVALLVILACVLW | TM domain |
| 11 | GWNPHLLLLLLLVIVFIPAFW | TM domain |
| 12 | MALPVTALLLPLALLLHAARP | CD8a signal |
| | | sequence |
| 13 | ATGGCCTTACCAGTGACCGCCTTGCTCCTGCCGCT | nucleotide |
| | GGCCTTGCTGCTCCACGCCGCCAGGCCGGATGTCG | sequence |
| | TGATGACCCAGACCCCCCTCAGCCTCCCAGTGTCC | encoding a |
| | CTCGGTGACCAGGCTTCTATTAGTTGCAGATCCAG | CAR |
| | CCAGTCCCTCGTGCACTCTAACGGTAATACCTACC | |
| | TGAGATGGTATCTCCAGAAGCCCGGACAGAGCCC | |
| | TAAGGTGCTGATCTACAAAGTCTCCAACCGGGTGT | |
| | CTGGAGTCCCTGACCGCTTCTCAGGGAGCGGTTCC | |
| | GGCACCGACTTCACCCTGAAGATCAACCGGGTGG | |
| | AGGCCGAAGACCTCGGCGTCTATTTCTGCTCTCAG | |
| | AGTACACATGTGCCCTGGACCTTCGGCGGAGGGA | |
| | CCAAGCTGGAGATCAAAAGCTCCGCAGACGATGC | |
| | CAAGAAAGATGCCGCTAAGAAAGACGATGCTAAG | |
| | AAAGACGATGCAAAGAAAGACGGTGGCGTGAAG | |
| | CTGGATGAAACCGGAGGAGGTCTCGTCCAGCCAG | |
| | GAGGAGCCATGAAGCTGAGTTGCGTGACCAGCGG | |
| | ATTCACCTTTGGGCACTACTGGATGAACTGGGTGC | |
| | GACAGTCCCCAGAGAAGGGGCTCGAATGGGTCGC | |
| | TCAGTTCAGGAACAAACCCTACAATTATGAGACA | |
| | TACTATTCAGACAGCGTGAAGGGCAGGTTTACTAT | |
| | CAGTAGAGACGATTCCAAATCTAGCGTGTACCTGC | |
| | AGATGAACAATCTCAGGGTCGAAGATACAGGCAT | |
| | CTACTATTGCACAGGGGCATCCTATGGTATGGAGT | |
| | ATCTCGGTCAGGGGACAAGCGTCACAGTCAGTTTC | |
| | GTGCCGGTCTTCCTGCCAGCGAAGCCCACCACGAC | |
| | GCCAGCGCCGCGACCACCAACACCGGCGCCCACC | |
| | ATCGCGTCGCAGCCCCTGTCCCTGCGCCCAGAGGC | |
| | GTGCCGGCCAGCGGCGGGGGGCGCAGTGCACACG | |
| | AGGGGGCTGGACTTCGCCTGTGATATCTACATCTG | |
| | GGCGCCCTTGGCCGGGACTTGTGGGGTCCTTCTCC | |
| | TGTCACTGGTTATCACCCTTTACTGCAACCACAGG | |
| | AACCGTTTCTCTGTTGTTAAACGGGGCAGAAAGA | |
| | AACTCCTGTATATATTCAAACAACCATTTATGAGA | |
| | CCAGTACAAACTACTCAAGAGGAAGATGGCTGTA | |
| | GCTGCCGATTTCCAGAAGAAGAAGAAGGAGGATG | |
| | TGAACTGAGAGTGAAGTTCAGCAGGAGCGCAGAC | |
| | GCCCCCGCGTACCAGCAGGGCCAGAACCAGCTCT | |
| | ATAACGAGCTCAATCTAGGACGAAGAGAGGAGTA | |
| | CGATGTTTTGGACAAGAGACGTGGCCGGGACCCT | |
| | GAGATGGGGGGAAAGCCGAGAAGGAAGAACCCT | |
| | CAGGAAGGCCTGTACAATGAACTGCAGAAAGATA | |
| | AGATGGCGGAGGCCTACAGTGAGATTGGGATGAA | |
| | AGGCGAGCGCCGGAGGGGCAAGGGGCACGATGG | |
| | CCTTTACCAGGGTCTCAGTACAGCCACCAAGGAC | |
| | ACCTACGACGCCCTTCACATGCAGGCCCTGCCCCC | |
| | TCGCTAA | |
| 14 | MALPVTALLLPLALLLHAARPDVVMTQTPLSLPVSL | CAR amino |
| | GDQASISCRSSQSLVHSNGNTYLR | acid |
| | WYLQKPGQSPKVLIYKVSNRVSGVPDRFSGSGSGTD | sequence |
| | FTLKINRVEAEDLGVYFCSQSTHV | |
| | PWTFGGGTKLEIKSSADDAKKDAAKKDDAKKDDA | |
| | KKDGGVKLDETGGGLVQPGGAMKLSC | |
| | VTSGFTFGHYWMNWVRQSPEKGLEWVAQFRNKPY | |
| | NYETYYSDSVKGRFTISRDDSKSSVY | |
| | LQMNNLRVEDTGIYYCTGASYGMEYLGQGTSVTVS | |
| | FVPVFLPAKPTTTPAPRPPTPAPTI | |
| | ASQPLSLRPEACRPAAGGAVHTRGLDFACDIYIWAP | |
| | LAGTCGVLLLSLVITLYCNHRNRF | |
| | SVVKRGRKKLLYIFKQPFMRPVQTTQEEDGCSCRFP | |
| | EEEEGGCELRVKFSRSADAPAYQQ | |
| | GQNQLYNELNLGRREEYDVLDKRRGRDPEMGGKPR | |
| | RKNPQEGLYNELQKDKMAEAYSEIG | |
| | MKGERRRGKGHDGLYQGLSTATKDTYDALHMQAL | |
| | PPR | |
| 15 | GCCACCATGGCCTTACCAGTGACCGCCTTGCTCCT | nucleotide |
| | GCCGCTGGCCTTGCTGCTCCACGCCGCCAGGCCGG | insert |
| | ATGTCGTGATGACCCAGACCCCCCTCAGCCTCCCA | |
| | GTGTCCCTCGGTGACCAGGCTTCTATTAGTTGCAG | |
| | ATCCAGCCAGTCCCTCGTGCACTCTAACGGTAATA | |
| | CCTACCTGAGATGGTATCTCCAGAAGCCCGGACA | |
| | GAGCCCTAAGGTGCTGATCTACAAAGTCTCCAACC | |
| | GGGTGTCTGGAGTCCCTGACCGCTTCTCAGGGAGC | |
| | GGTTCCGGCACCGACTTCACCCTGAAGATCAACCG | |
| | GGTGGAGGCCGAAGACCTCGGCGTCTATTTCTGCT | |
| | CTCAGAGTACACATGTGCCCTGGACCTTCGGCGGA | |
| | GGGACCAAGCTGGAGATCAAAAGCTCCGCAGACG | |
| | ATGCCAAGAAAGATGCCGCTAAGAAAGACGATGC | |
| | TAAGAAAGACGATGCAAAGAAAGACGGTGGCGTG | |
| | AAGCTGGATGAAACCGGAGGAGGTCTCGTCCAGC | |
| | CAGGAGGAGCCATGAAGCTGAGTTGCGTGACCAG | |
| | CGGATTCACCTTTGGGCACTACTGGATGAACTGGG | |
| | TGCGACAGTCCCCAGAGAAGGGGCTCGAATGGGT | |
| | CGCTCAGTTCAGGAACAAACCCTACAATTATGAG | |
| | ACATACTATTCAGACAGCGTGAAGGGCAGGTTTA | |
| | CTATCAGTAGAGACGATTCCAAATCTAGCGTGTAC | |
| | CTGCAGATGAACAATCTCAGGGTCGAAGATACAG | |
| | GCATCTACTATTGCACAGGGGCATCCTATGGTATG | |
| | GAGTATCTCGGTCAGGGGACAAGCGTCACAGTCA | |
| | GTTTCGTGCCGGTCTTCCTGCCAGCGAAGCCCACC | |
| | ACGACGCCAGCGCCGCGACCACCAACACCGGCGC | |
| | CCACCATCGCGTCGCAGCCCCTGTCCCTGCGCCCA | |
| | GAGGCGTGCCGGCCAGCGGCGGGGGGCGCAGTGC | |
| | ACACGAGGGGGCTGGACTTCGCCTGTGATATCTAC | |
| | ATCTGGGCGCCCTTGGCCGGGACTTGTGGGGTCCT | |
| | TCTCCTGTCACTGGTTATCACCCTTTACTGCAACC | |
| | ACAGGAACCGTTTCTCTGTTGTTAAACGGGGCAGA | |
| | AAGAAACTCCTGTATATATTCAAACAACCATTTAT | |
| | GAGACCAGTACAAACTACTCAAGAGGAAGATGGC | |
| | TGTAGCTGCCGATTTCCAGAAGAAGAAGAAGGAG | |
| | GATGTGAACTGAGAGTGAAGTTCAGCAGGAGCGC | |
| | AGACGCCCCCGCGTACCAGCAGGGCCAGAACCAG | |
| | CTCTATAACGAGCTCAATCTAGGACGAAGAGAGG | |
| | AGTACGATGTTTTGGACAAGAGACGTGGCCGGGA | |
| | CCCTGAGATGGGGGGAAAGCCGAGAAGGAAGAA | |
| | CCCTCAGGAAGGCCTGTACAATGAACTGCAGAAA | |
| | GATAAGATGGCGGAGGCCTACAGTGAGATTGGGA | |
| | TGAAAGGCGAGCGCCGGAGGGGCAAGGGGCACG | |
| | ATGGCCTTTACCAGGGTCTCAGTACAGCCACCAAG | |
| | GACACCTACGACGCCCTTCACATGCAGGCCCTGCC | |
| | CCCTCGCTAA | |
| 16 | DVVMTQTPLSLPVSLGDQASISCRSSQSLVHSNGNT | CAR amino |
| | YLRWYLQKPGQSPKVLIYKVSNRVSGVPDRFSGSGS | acid |
| | GTDFTLKINRVEAEDLGVYFCSQSTHV | sequence |
| | PWTFGGGTKLEIKSSADDAKKDAAKKDDAKKDDA | without the |
| | KKDGGVKLDETGGGLVQPGGAMKLSC | signal |
| | VTSGFTFGHYWMNWVRQSPEKGLEWVAQFRNKPY | peptide |
| | NYETYYSDSVKGRFTISRDDSKSSVY | |
| | LQMNNLRVEDTGIYYCTGASYGMEYLGQGTSVTVS | |
| | FVPVFLPAKPTTTPAPRPPTPAPTI | |
| | ASQPLSLRPEACRPAAGGAVHTRGLDFACDIYIWAP | |
| | LAGTCGVLLLSLVITLYCNHRNRF | |
| | SVVKRGRKKLLYIFKQPFMRPVQTTQEEDGCSCRFP | |
| | EEEEGGCELRVKFSRSADAPAYQQ | |
| | GQNQLYNELNLGRREEYDVLDKRRGRDPEMGGKPR | |
| | RKNPQEGLYNELQKDKMAEAYSEIG | |
| | MKGERRRGKGHDGLYQGLSTATKDTYDALHMQAL | |
| | PPR | |
| 17 | MALPVTALLLPLALLLHAARP | CAR amino |
| | | acid |
| | | sequence |
| | | signal |
| | | peptide |
| 18 | GGCCGAGATGTCTCGCTCCG | B2M gRNA |
| 19 | CACGCAGGTCTGGCCGCGCT | FKBP12 |
| | | gRNA |
| 20 | TGCTGAGCCGATGCGCGCGG | FKBP12 |
| | | gRNA |
| 21 | CTCACCGTCTCCTGGGGAGA | FKBP12 |
| | | gRNA |
| 22 | TACCCAGAGAATGGAGAAACCCTGCAGGGAATTC | B2M 5′ |
| | CCAAGCTGTAGTTATAAACAGAAGTTCTCCTTCTG | homology |
| | CTAGGTAGCATTCAAAGATCTTAATCTTCTGGGTT | arm |
| | TCCGTTTTCTCGAATGAAAAATGCAGGTCCGAGCA | |
| | GTTAACTGGCTGGGGCACCATTAGCAAGTCACTTA | |
| | GCATCTCTGGGGCCAGTCTGCAAAGCGAGGGGGC | |
| | AGCCTTAATGTGCCTCCAGCCTGAAGTCCTAGAAT | |
| | GAGCGCCCGGTGTCCCAAGCTGGGGCGCGCACCC | |
| | CAGATCGGAGGGCGCCGATGTACAGACAGCAAAC | |
| | TCACCCAGTCTAGTGCATGCCTTCTTAAACATCAC | |
| | GAGACTCTAAGAAAAGGAAACTGAAAACGGGAA | |
| | AGTCCCTCTCTCTAACCTGGCACTGCGTCGCTGGC | |
| | TTGGAGACAGGTGACGGTCCCTGCGGGCCTTGTCC | |
| | TGATTGGCTGGGCACGCGTTTAATATAAGTGGAG | |
| | GCGTCGCGCTGGCGGGC | |
| 23 | TGGCCTTAGCTGTGCTCGCGCTACTCTCTCTTTCTG | B2M 3′ |
| | GCCTGGAGGCTATCCAGCGTGAGTCTCTCCTACCC | homology |
| | TCCCGCTCTGGTCCTTCCTCTCCCGCTCTGCACCCT | arm |
| | CTGTGGCCCTCGCTGTGCTCTCTCGCTCCGTGACTT | |
| | CCCTTCTCCAAGTTCTCCTTGGTGGCCCGCCGTGG | |
| | GGCTAGTCCAGGGCTGGATCTCGGGGAAGCGGCG | |
| | GGGTGGCCTGGGAGTGGGGAAGGGGGTGCGCACC | |
| | CGGGACGCGCGCTACTTGCCCCTTTCGGCGGGGA | |
| | GCAGGGGAGACCTTTGGCCTACGGCGACGGGAGG | |
| | GTCGGGACAAAGTTTAGGGCGTCGATAAGCGTCA | |
| | GAGCGCCGAGGTTGGGGGAGGGTTTCTCTTCCGCT | |
| | CTTTCGCGGGGCCTCTGGCTCCCCCAGCGCAGCTG | |
| | GAGTGGGGGACGGGTAGGCTCGTCCCAAAGGCGC | |
| | GGCGCTGAGGTTTGTGAACGCGTGGAGGGGCGCT | |
| | TGGGGTCTGGGGGA | |
| 24 | cgtgaggctccggtgcccgtcagtgggcagagcgcacategcccacagtccccg | EF1alpha |
| | agaagttggggggaggggtcggcaattgaaccggtgcctagagaaggtggcgc | promoter |
| | ggggtaaactgggaaagtgatgtcgtgtactggctccgcctttttcccgagggtgg | |
| | gggagaaccgtatataagtgcagtagtcgccgtgaacgttctttttcgcaacgggttt | |
| | gccgccagaacacaggtaagtgccgtgtgtggttcccgcgggcctggcctctttac | |
| | gggttatggcccttgcgtgccttgaattacttccacgcccctggctgcagtacgtgat | |
| | tcttgatcccgagcttcgggttggaagtggggggagagttcgaggccttgcgctta | |
| | aggagccccttcgcctcgtgcttgagttgaggcctggcctgggcgctggggccgc | |
| | cgcgtgcgaatctggtggcaccttcgcgcctgtctcgctgctttcgataagtctctag | |
| | ccatttaaaatttttgatgacctgctgcgacgctttttttctggcaagatagtcttgtaaa | |
| | tgcgggccaagatctgcacactggtatttcggtttttggggccgcgggcggcgac | |
| | ggggcccgtgcgtcccagcgcacatgttcggcgaggcggggcctgcgagcgcg | |
| | gccaccgagaatcggacgggggtagtctcaagctggccggcctgctctggtgcct | |
| | ggcctcgcgccgccgtgtatcgccccgccctgggcggcaaggctggcccggtc | |
| | ggcaccagttgcgtgagcggaaagatggccgcttcccggccctgctgcagggag | |
| | ctcaaaatggaggacgcggcgctcgggagagcggggggtgagtcacccacac | |
| | aaaggaaaagggcctttccgtcctcagccgtcgcttcatgtgactccacggagtac | |
| | cgggcgccgtccaggcacctcgattagttctcgagcttttggagtacgtcgtctttag | |
| | gttggggggaggggttttatgcgatggagtttccccacactgagtgggtggagact | |
| | gaagttaggccagcttggcacttgatgtaattctccttggaatttgccctttttgagttt | |
| | ggatcttggttcattctcaagcctcagacagtggttcaaagtttttttcttccatttcagg | |
| | tgtcgtgaa | |
| 25 | CGTGAGGCTCCGGTGCCCGTCAGTGGGCAGAGCG | EF1alpha |
| | CACATCGCCCACAGTCCCCGAGAAGTTGGGGGGA | promoter |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| 26 | CGTGAGGCTCCGGTGCCCGTCAGTGGGCAGAGCG | EF1alpha |
| | CACATCGCCCACAGTCCCCGAGAAGTTGGGGGGA | promoter |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCACTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACCTGGCTGCAGTACGTGATTCTTGATCCCGAGC | |
| | TTCGGGTTGGAAGTGGGTGGGAGAGTTCGTGGCC | |
| | TTGCGCTTAAGGAGCCCCTTCGCCTCGTGCTTGAG | |
| | TTGTGGCCTGGCCTGGGCGCTGGGGCCGCCGCGTG | |
| | CGAATCTGGTGGCACCTTCGCGCCTGTCTCGCTGC | |
| | TTTCGATAAGTCTCTAGCCATTTAAAATTTTTGAT | |
| | GACCTGCTGCGACGCTTTTTTTCTGGCAAGATAGT | |
| | CTTGTAAATGCGGGCCAAGATCAGCACACTGGTA | |
| | TTTCGGTTTTTGGGGCCGCGGGCGGCGACGGGGCC | |
| | CGTGCGTCCCAGCGCACATGTTCGGCGAGGCGGG | |
| | GCCTGCGAGCGCGGCCACCGAGAATCGGACGGGG | |
| | GTAGTCTCAAGCTGCCCGGCCTGCTCTGGTGCCTG | |
| | GCCTCGCGCCGCCGTGTATCGCCCCGCCCTGGGCG | |
| | GCAAGGCTGGCCCGGTCGGCACCAGTTGCGTGAG | |
| | CGGAAAGATGGCCGCTTCCCGGCCCTGCTGCAGG | |
| | GAGCACAAAATGGAGGACGCGGCGCTCGGGAGA | |
| | GCGGGCGGGTGAGTCACCCACACAAAGGAAAAGG | |
| | GCCTTTCCGTCCTCAGCCGTCGCTTCATGTGACTC | |
| | CACGGAGTACCGGGCGCCGTCCAGGCACCTCGAT | |
| | TAGTTCTCCAGCTTTTGGAGTACGTCGTCTTTAGG | |
| | TTGGGGGGAGGGGTTTTATGCGATGGAGTTTCCCC | |
| | ACACTGAGTGGGTGGAGACTGAAGTTAGGCCAGC | |
| | TTGGCACTTGATGTAATTCTCCTTGGAATTTGCCCT | |
| | TTTTGAGTTTGGATCTTGGTTCATTCTCAAGCCTCA | |
| | GACAGTGGTTCAAAGTTTTTTTCTTCCATTTCAGGT | |
| | GTCGTGAAAACTACCCCTAAAAGCCAAA | |
| 27 | gaacagagaaacaggagaatatgggccaaacaggatatctgtggtaagcagttcc | MND |
| | tgccccggctcagggccaagaacagttggaacagcagaatatgggccaaacag | promoter |
| | gatatctgtggtaagcagttcctgccccggctcagggccaagaacagatggtccc | |
| | cagatgcggtcccgccctcagcagtttctagagaaccatcagatgtttccagggtg | |
| | ccccaaggacctgaaatgaccctgtgccttatttgaactaaccaatcagttcgcttct | |
| | cgcttctgttcgcgcgcttctgctccccgagctctatataagcagagctcgtttagtg | |
| | aaccgtcagatc | |
| 28 | GVQVETISPGDGRTFPKRGQTCVVHYTGMLEDGKK | RACR (g) |
| | FDSSRDRNKPFKFMLGKQEVIRGWEEGVAQMSVGQ | |
| | RAKLTISPDYAYGATGHPGIIPPHATLVFDVELLKLG | |
| | EGSNTSKENPFLFALEAVVISVGSMGLIISLLCVYFW | |
| | LERTMPRIPTLKNLEDLVTEYHGNFSAWSGVSKGLA | |
| | ESLQPDYSERLCLVSEIPPKGGALGEGPGASPCNQHS | |
| | PYWAPPCYTLKPET | |
| 29 | MPLGLLWLGLALLGALHAQA | Signal |
| | | peptide |
| 30 | GVQVETISPGDGRTFPKRGQTCVVHYTGMLEDGKK | FKBP |
| | FDSSRDRNKPFKFMLGKQEVIRGWEEGVAQMSVGQ | domain |
| | RAKLTISPDYAYGATGHPGIIPPHATLVFDVELLKLG | (FKBP12) |
| | E | |
| 31 | GSNTSKENPFLFALEAVVISVGSMGLIISLLCVYFWL | IL2RG |
| | | transmembrane |
| | | domain |
| | | and |
| | | ectodomain |
| | | portion |
| 32 | CGTGAGGCTCCGGTGCCCGTCAGTGGGCAGAGCG | RACR |
| | CACATCGCCCACAGTCCCCGAGAAGTTGGGGGGA | |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| | GCCACC | |
| | ATGCCACTGGGACTGCTGTGGCTGGGACTGGCCCT | |
| | GCTGGGCGCCCTGCACGCCCAGGCCGGCGTGCAG | |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| | GGAAGCGGAGCGACGAATTTTAGTCTACTGAAAC | |
| | AAGCGGGAGACGTGGAGGAAAACCCTGGACCT | |
| | ATGGCACTGCCAGTGACCGCCCTGCTGCTGCCACT | |
| | GGCCCTGCTGCTGCACGCAGCACGCCCCATCCTGT | |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| | TAAAC | |
| | AATAAAAGATCTTTATTTTCATTAGATCTGTGTGT | |
| | TGGTTTTTTGTGTG | |
| 33 | MALPVTALLLPLALLLHAARPILWHEMWHEGLEEA | RACR (b) |
| | SRLYFGERNVKGMFEVLEPLHAMMERGPQTLKETS | |
| | FNQAYGRDLMEAQEWCRKYMKSGNVKDLLQAWD | |
| | LYYHVFRRISKGKDTIPWLGHLLVGLSGAFGFIILVY | |
| | LLINCRNTGPWLKKVLKCNTPDPSKFFSQLSSEHGG | |
| | DVQKWLSSPFPSSSFSPGGLAPEISPLEVLERDKVTQL | |
| | LLQQDKVPEPASLSSNHSLTSCFTNQGYFFFHLPDAL | |
| | EIEACQVYFTYDPYSEEDPDEGVAGAPTGSSPQPLQP | |
| | LSGEDDAYCTFPSRDDLLLFSPSLLGGPSPPSTAPGGS | |
| | GAGEERMPPSLQERVPRDWDPQPLGPPTPGVPDLVD | |
| | FQPPPELVLREAGEEVPDAGPREGVSFPWSRPPGQGE | |
| | FRALNARLPLNTDAYLSLQELQGQDPTHLV | |
| 34 | MALPVTALLLPLALLLHAARP | CD8a Signal |
| | | peptide |
| 35 | GKDTIPWLGHLLVGLSGAFGFIILVYLLI | IL2RB |
| | | transmembrane |
| | | domain |
| | | and |
| | | ectodomain |
| | | portion |
| 36 | IPWLGHLLVGLSGAFGFIILVYLLI | IL2RB |
| | | transmembrane |
| | | domain |
| 37 | ATGCCACTGGGACTGCTGTGGCTGGGACTGGCCCT | RACR (g) |
| | GCTGGGCGCCCTGCACGCCCAGGCCGGCGTGCAG | Nucleotides |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| 38 | ATGGCACTGCCAGTGACCGCCCTGCTGCTGCCACT | RACR (b) |
| | GGCCCTGCTGCTGCACGCAGCACGCCCCATCCTGT | nucleotides |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| 39 | AATAAAAGATCTTTATTTTCATTAGATCTGTGTGT | Synthetic |
| | TGGTTTTTTGTGTG | polyA |
| 40 | TACCCAGAGAATGGAGAAACCCTGCAGGGAATTC | RACR_B2M |
| | CCAAGCTGTAGTTATAAACAGAAGTTCTCCTTCTG | SSODN |
| | CTAGGTAGCATTCAAAGATCTTAATCTTCTGGGTT | |
| | TCCGTTTTCTCGAATGAAAAATGCAGGTCCGAGCA | |
| | GTTAACTGGCTGGGGCACCATTAGCAAGTCACTTA | |
| | GCATCTCTGGGGCCAGTCTGCAAAGCGAGGGGGC | |
| | AGCCTTAATGTGCCTCCAGCCTGAAGTCCTAGAAT | |
| | GAGCGCCCGGTGTCCCAAGCTGGGGCGCGCACCC | |
| | CAGATCGGAGGGCGCCGATGTACAGACAGCAAAC | |
| | TCACCCAGTCTAGTGCATGCCTTCTTAAACATCAC | |
| | GAGACTCTAAGAAAAGGAAACTGAAAACGGGAA | |
| | AGTCCCTCTCTCTAACCTGGCACTGCGTCGCTGGC | |
| | TTGGAGACAGGTGACGGTCCCTGCGGGCCTTGTCC | |
| | TGATTGGCTGGGCACGCGTTTAATATAAGTGGAG | |
| | GCGTCGCGCTGGCGGGC | |
| | CGTGAGGCTCCGGTGCCCGTCAGTGGGCAGAGCG | |
| | CACATCGCCCACAGTCCCCGAGAAGTTGGGGGGA | |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| | GCCACC | |
| | ATGCCACTGGGACTGCTGTGGCTGGGACTGGCCCT | |
| | GCTGGGCGCCCTGCACGCCCAGGCCGGCGTGCAG | |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| | GGAAGCGGAGCGACGAATTTTAGTCTACTGAAAC | |
| | AAGCGGGAGACGTGGAGGAAAACCCTGGACCT | |
| | ATGGCACTGCCAGTGACCGCCCTGCTGCTGCCACT | |
| | GGCCCTGCTGCTGCACGCAGCACGCCCCATCCTGT | |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| | TAAAC | |
| | AATAAAAGATCTTTATTTTCATTAGATCTGTGTGT | |
| | TGGTTTTTTGTGTG | |
| | TGGCCTTAGCTGTGCTCGCGCTACTCTCTCTTTCTG | |
| | GCCTGGAGGCTATCCAGCGTGAGTCTCTCCTACCC | |
| | TCCCGCTCTGGTCCTTCCTCTCCCGCTCTGCACCCT | |
| | CTGTGGCCCTCGCTGTGCTCTCTCGCTCCGTGACTT | |
| | CCCTTCTCCAAGTTCTCCTTGGTGGCCCGCCGTGG | |
| | GGCTAGTCCAGGGCTGGATCTCGGGGAAGCGGCG | |
| | GGGTGGCCTGGGAGTGGGGAAGGGGGTGCGCACC | |
| | CGGGACGCGCGCTACTTGCCCCTTTCGGCGGGGA | |
| | GCAGGGGAGACCTTTGGCCTACGGCGACGGGAGG | |
| | GTCGGGACAAAGTTTAGGGCGTCGATAAGCGTCA | |
| | GAGCGCCGAGGTTGGGGGAGGGTTTCTCTTCCGCT | |
| | CTTTCGCGGGGCCTCTGGCTCCCCCAGCGCAGCTG | |
| | GAGTGGGGGACGGGTAGGCTCGTCCCAAAGGCGC | |
| | GGCGCTGAGGTTTGTGAACGCGTGGAGGGGCGCT | |
| | TGGGGTCTGGGGGA | |
| 41 | GGAAGCGGAGCGACGAATTTTAGTCTACTGAAAC | PQR linker |
| | AAGCGGGAGACGTGGAGGAAAACCCTGGACCT | nucleotide |
| 42 | GSGATNFSLLKQAGDVEENPGP | PQR linker |
| 43 | VKQTLNFDLLKLAGDVESNPGP | F2A |
| 44 | QCTNYALLKLAGDVESNPGP | E2A |
| 45 | LEGGGEGRGSLLTCGDVEENPGPR | T2A |
| 46 | EGRGSLLTCGDVEENPGP | T2A |
| 47 | GSGATNFSLLKQAGDVEENPGP | P2A |
| 48 | GVQVETISPGDGRTFPKRGQTCVVHYTGMLEDGKK | FKBP |
| | FDSSRDRNKPFKFMLGKQEVIRGWE | domain |
| | EGVAQMSVGQRAKLTISPDYAYGATGHPGIIPPHAT | |
| | LVFDVELLKL | |
| 49 | GVQVETISPGDGRTFPKRGQTCVVHYTGMLEDGKK | FKBP |
| | FDSSRDRNKPFKFMLGKQEVIRGWEEGVAQMSVGQ | domain |
| | RAKLTISPDYAYGATGHPGIIPPHATLVFDVELLKLG | |
| | E | |
| 50 | MEMWHEGLEEASRLYFGERNVKGMFEVLEPLHAM | FRB domain |
| | MERGPQTLKETSFNQAYGRDLMEAQEWCRKYMKS | |
| | GNVKDLLQAWDLYYHVFRRISK | |
| 51 | KLSPRVKRIFYQNVPSPAMFFQPLYSVHNGNFQ | IL-9RA |
| | TWMGAHGAGVLLSQDCAGTPQGALEPCVQEA | |
| | TALLTCGPARPWKSVALEEEQEGPGTRLPGNLS | |
| | SEDVLPAGCTEWRVQTLAYLPQEDWAPTSLTRP | |
| | APPDSEGSRSSSSSSSSNNNNYCALGCYGGWHL | |
| | SALPGNTQSSGPIPALACGLSCDHQGLETQQGV | |
| | AWVLAGHCQRPGLHEDLQGMLLPSVLSKARS | |
| | WTF | |

What is claimed is:

1. An engineered cytotoxic innate lymphoid (CIL) cell engineered for controlled expansion and/or activity, the engineered lymphocyte or progenitor cell comprising a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic alpha or beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, an interleukin-21 receptor subunit beta (IL-21RB) intracellular domain or an interleukin-9 receptor subunit alpha (IL-9RA) intracellular domain;
wherein binding of the non-physiological ligand to the synthetic cytokine receptor activates the synthetic cytokine receptor in the engineered CIL cell to induce expansion and/or activation of the engineered CIL cell in a cell population.

2. The engineered CIL cell of claim 1,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

3. The engineered CIL cell of claim 1 or claim 2, wherein the first dimerization domain and the second dimerization domain are extracellular domains.

4. The engineered CIL cell of any of claims 1-3, wherein the synthetic gamma chain polypeptide comprises, in N- to C-terminal order, the first dimerization domain, the first transmembrane domain, and the interleukin-2 receptor subunit gamma (IL-2RG) intracellular domain, and the synthetic beta chain polypeptide comprises, in N- to C-terminal order, the second dimerization domain, the second transmembrane domain, and the intracellular domain.

5. The engineered CIL cell of any of claims 1-4, wherein the IL-2RG intracellular domain comprises a polypeptide sequence at least 95% identical to SEQ ID NO: 1, or a polypeptide sequence as set forth in SEQ ID NO: 1.

6. The engineered CIL cell of any of claims 1-5, wherein the first transmembrane domain comprises the IL-2RG transmembrane domain.

7. The engineered CIL cell of claim 6, wherein the IL-2RG transmembrane domain comprises a polypeptide sequence at least 95% identical to SEQ ID NO: 8 or 31, or a polypeptide sequence as set forth in SEQ ID NO: 8 or 31.

8. The engineered CIL cell of any of claims 1-7, wherein the beta chain intracellular domain comprises the IL-2RB intracellular domain.

9. The CIL cell of claim 8, wherein the IL-2RB intracellular domain comprises a polypeptide sequence at least 95% identical to SEQ ID NO: 2, or a polypeptide sequence as set forth in SEQ ID NO: 2.

10. The engineered CIL cell of any of claims 1-7, wherein the beta chain intracellular domain comprises the IL-7RB intracellular domain.

11. The engineered CIL cell of any of claims 1-7, wherein the IL-7RB intracellular domain comprises a polypeptide sequence at least 95% identical to SEQ ID NO: 3, or a polypeptide sequence as set forth in SEQ ID NO: 3.

12. The engineered CIL cell of any of claims 1-7, wherein the beta chain intracellular domain comprises the IL-21RB intracellular domain.

13. The engineered CIL cell of claim 12, wherein the IL-21RB intracellular domain comprises a polypeptide sequence at least 95% identical to SEQ ID NO: 4, or a polypeptide sequence as set forth in SEQ ID NO: 4.

14. The engineered CIL cell of any of claims 2-7, wherein the synthetic alpha chain polypeptide comprises the IL-9RA intracellular domain.

15. The engineered CIL cell of claim 14, wherein the IL-9RA intracellular domain comprises a polypeptide sequence at least 95% identical to SEQ ID NO: 51, or a polypeptide sequence as set forth in SEQ ID NO: 51.

16. The engineered CIL cell of any of claims 1-13, wherein the second transmembrane domain comprises a transmembrane domain from the same polypeptide as the intracellular domain.

17. The engineered CIL cell of claims 1-9 and 16, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

18. The engineered CIL cell of any of claims 1-9, 16 and 17, wherein:
the synthetic gamma chain polypeptide contains an IL-2RG TM domain comprising the sequence set forth in SEQ ID NO: 8 or 31 and an IL-2RG intracellular domain comprising the sequence set forth in SEQ ID NO:1; and
the synthetic beta chain polypeptide contains an IL-2RB TM domain comprising the sequence set forth in SEQ ID NO: 35 or 36 and an IL-2RB intracellular domain comprising the sequence set forth in SEQ ID NO:2.

19. The engineered CIL cell of any of claims 1-18, wherein the first dimerization domain and the second dimerization domain are heterodimerization domains selected from FK506-Binding Protein of size 12 kD (FKBP) and a FKBP12-rapamycin binding (FRB) domain; and/or
wherein the non-physiological ligand is rapamycin or a rapalog.

20. The engineered CIL cell of claim 19, wherein the first dimerization domain is FKBP and the second dimerization domain is FRB.

21. The engineered CIL cell of claim 19, wherein the first dimerization domain is FRB and the second dimerization domain is FKBP.

22. The engineered CIL cell of any of claims 19-21, wherein the FRB domain comprises a polypeptide sequence at least 95% identical to SEQ ID NO: 6 or SEQ ID NO: 7.

23. The engineered CIL cell of any of claims 19-21, wherein the FRB domain comprises the polypeptide sequence set forth in SEQ ID NO: 6 or SEQ ID NO: 7.

24. The engineered CIL cell of any of claims 1-18, wherein the first dimerization domain and the second dimerization domain are heterodimerization domains selected from selected from FK506-Binding Protein of size 12 kD (FKBP) and a calcineurin domain; and/or
wherein the non-physiological ligand is FK506 or an analogue thereof.

25. The engineered CIL cell of any of claims 1-19, wherein the FKBP domain comprises a polypeptide sequence at least 95% identical to SEQ ID NO: 5 or SEQ ID NO: 30.

26. The engineered CIL cell of any of claims 1-19, wherein the FKBP domain comprises the polypeptide sequence set forth in SEQ ID NO: 5 or SEQ ID NO: 30.

27. The engineered CIL cell of any of claims 1-9 and 16-26, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

28. The engineered CIL cell of any of claims 1-9 and 16-27, wherein the synthetic gamma chain polypeptide has the sequence of amino acids set forth in SEQ ID NO: 28 and the synthetic beta chain polypeptide has the sequence of amino acids set forth in SEQ ID NO:33.

29. The engineered CIL cell of any of claims 1-18, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

30. The engineered CIL cell of any of claims 1-29, wherein the CIL cell is resistant to rapamycin-mediated mTOR inhibition.

31. The engineered CIL cell of any of claims 1-30, wherein the cytotoxic innate lymphoid cells express a cytosolic polypeptide that binds to the non-physiological ligand, optionally wherein the cytosolic polypeptide is a cytosolic FRB domain.

32. The engineered CIL cell of any of claims 1-31, wherein the non-physiological ligand is rapamycin or a rapalog, and the cytotoxic innate lymphoid cells express a cytosolic FRB domain or variant thereof.

33. The engineered CIL cell of claim 26 or claim 27, wherein the cytosolic FRB domain comprises a polypeptide sequence at least 95% identical to SEQ ID NO:
6 or SEQ ID NO: 7.

34. The engineered CIL cell of claim 26 or claim 27, wherein the cytosolic FRB domain comprises a polypeptide sequence at least 98% identical to SEQ ID NO:
6 or SEQ ID NO: 7.

35. The engineered CIL cell of any of claims 1-34, wherein the CIL cell comprises a disrupted FKBP12 gene that reduces expression of FKBP12.

36. The engineered CIL cell of any of claims 1-35, wherein the CIL cell comprises knock out of the FKBP12 gene.

37. The engineered CIL cell of any of claims 1-36, wherein the CIL cells comprise a nucleotide sequence encoding the synthetic cytokine receptor inserted into the genome of the CIL cell.

38. The engineered CIL cell of claim 37, wherein the nucleotide sequence encoding the synthetic cytokine receptor is inserted into a non-target locus in the genome of the CIL cell.

39. The engineered CIL cell of claim 37, wherein the nucleotide sequence encoding the synthetic cytokine receptor is inserted into an endogenous gene of the CIL cell.

40. The engineered CIL cell of claim 39, wherein the insertion reduces expression of the endogenous gene in the locus.

41. The engineered CIL cell of claim 39 or claim 40, wherein the insertion knocks out the endogenous gene in the locus.

42. The engineered CIL cell of any of claims 39-41 wherein the insertion is by homology-directed repair.

43. The engineered CIL cell of any of claims 39-42, wherein the endogenous gene is a housekeeping gene, a blood-lineage specific loci or an immune-related gene.

44. The engineered CIL cell of claim 43, wherein the endogenous gene is a housekeeping gene, and the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).

45. The engineered CIL cell of claim 43, wherein the endogenous gene is a blood-lineage specific loci and the blood-lineage specific loci is selected from protein tyrosine phosphatase receptor type C (PTPRC), IL2RG, and IL2RB.

46. The engineered CIL cell of claim 43, wherein the immune-related gene is selected from a beta-2-microglobulin (B2M) gene, a T cell receptor alpha constant (TRAC) gene, and a signal regulatory protein alpha (SIRPA) gene.

47. The engineered CIL cell of any one of claims 1-46, wherein the CIL cells comprise a B2M knockout.

48. The engineered CIL cell of any of claims 1-47, wherein the CIL cells comprise a B2M knockout and a FKBP12 knockout.

49. The engineered CIL cell of any one of claims 1-48, comprising a chimeric antigen receptor (CAR).

50. The engineered CIL cell of claim 49, wherein the CAR is an anti-FITC CAR.

51. A cell population comprising engineered CIL cells of any of claims 1-50.

52. A method of genetically engineering CIL cells to express a synthetic cytokine receptor, comprising:
contacting a population of CIL cells with (i) a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand, thereby inserting the nucleotide sequence into the endogenous gene;
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

53. The method of claim 52, wherein the nucleotide sequence is inserted via homology directed repair (HDR).

54. The method of claim 53, wherein the vector comprises a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream.

55. The method of claim 52, wherein the nucleotide sequence is inserted via non-homologous end joining (NHEJ).

56. The method of any one of claims 52-55, wherein the RNA-guided endonuclease is selected from a Cas endonuclease, a Mad endonuclease, and a Cpf1 endonuclease.

57. The method of any one of claims 52-56, wherein the RNA-guided endonuclease is Cas9 or Mad7.

58. The method of any of claims 52-57, wherein the endogenous gene is selected from B2M, TRAC and SIRPA.

59. The method of any of claims 52-58, wherein the endogenous gene is B2M.

60. The method of any of claims 52-59, wherein the gRNA comprises the sequence set forth in SEQ ID NO:18.

61. The method of any of claims 54-60, wherein the nucleotide sequence homologous with a region located upstream of the target site comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the nucleotide sequence homologous with a region located downstream comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23.

62. The method of any of claims 52-61, wherein the nucleotide sequence encoding the synthetic cytokine receptor comprises a first nucleic acid sequence encoding a gamma chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37, and a second nucleic acid sequence encoding a beta chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38.

63. The method of claim 62, wherein the first nucleic acid sequence and second nucleic acid sequence are separated by a cleavable linker or an IRES.

64. The method of claim 63, wherein the cleavable linker is a protein quantitation reporter linker (PQR), optionally set forth in SEQ ID NO:42.

65. The method of any of claims 52-64, wherein the nucleotide sequence encoding a synthetic cytokine receptor for a non-physiological ligand is under the operable control of a heterologous promoter.

66. The method of claim 65, wherein the heterologous promoter is the EF1α promoter or the MND promoter.

67. The method of any of claims 52-65, wherein the nucleotide sequence encoding the synthetic cytokine receptor comprises a polyadenylation sequence.

68. The method of any of claims 52-67, wherein the recombinant vector comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40.

69. The method of any one of claims 52-68, comprising engineering the population of CIL cells to be resistant to rapamycin-mediated mTOR inhibition.

70. The method of claim 69, wherein engineering the population of CIL cells to be resistant to rapamycin comprises knocking out a FKBP12 gene.

71. The method of claim 69, wherein the method further comprising contacting the population of CIL cells with a guide RNA (gRNA) targeting a target site in the FKBP12 gene.

72. The method of claim 69, wherein the gRNA comprises one or more gRNA selected from a gRNA comprising the sequence set forth in SEQ ID NO: 19, SEQ ID NO:20 or SEQ ID NO:21.

73. The method of claim 72, wherein the gRNA is a pool of gRNA comprising 2 or 3 gRNA.

74. The method of any of claims 52-73, further comprising introducing into the population of CIL cells a chimeric antigen receptor (CAR).

75. The method of claim 74, wherein the CAR is an anti-FITC CAR.

76. A cell population produced by the method of any one of claims 52-75.

77. A pharmaceutical composition comprising the cell population of any one of claims 1-37 and 76.

78. A method of expanding an engineered cytotoxic innate lymphoid cell (CIL), the method comprising contacting a CIL of any of claims 1-50 with the non-physiological ligand of the synthetic cytokine receptor.

79. A method of treating a cancer in a subject, comprising administering to the subject an effective amount of the engineered CIL of any one of claims 1-50, the cell population of claim 76, or the pharmaceutical composition of claim 77.

80. A method of treating a cancer in a subject, comprising administering to the subject an effective amount of the engineered CIL of any of claims 1-50, the cell population of claim 76, or the pharmaceutical composition of claim 77.

81. The method of claim 80, wherein the subject has not been administered a lymphodepleting therapy prior to the administering the CIL, population of CILs or the pharmaceutical composition.

82. The method of claim 80 or claim 81, wherein the CIL express a CAR targeting cancer cells in the subject.

83. The method of claim 82, wherein the CAR is an anti-FITC CAR, and the subject has been administered a FITC-ligand to tag a cancer cell in the subject, wherein the ligand specifically binds a molecule expressed on a tumor.

84. The method of claim 83, wherein the FITC-ligand is FITC-folate.

85. The method of claim 84, wherein the non-physiological ligand is rapamycin or a rapamycin analog, optionally wherein the rapamycin analog is rapalog.

86. The method of any of claims 78-85, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

87. The method of any of claims 78-85, wherein the non-physiological ligand is contacted at a concentration of at or about 10 nM.

88. The method of any of claims 78-85, wherein the non-physiological ligand is contacted at a concentration of at or about 100 nM

89. A method of killing or inhibiting the proliferation of cancer cells, comprising contacting cancer cells with the engineered CIL of any of claims 1-50, the cell population of claim 76, or the pharmaceutical composition of claim 77.

90. The method of killing or inhibiting the proliferation of cancer cells of claim 89, wherein the method is performed in vivo in a subject.

91. A kit comprising the engineered CIL of any of claims 1-50, the cell population of claim 76, or the pharmaceutical composition of claim 77 and instructions for administering to a subject in need thereof.

92. The kit of claim 91, further comprising a container comprising the non-physiological ligand and instructions for administering the non-physiological ligand to the subject after administration of the cell population.

93. The kit of claim 91 or 92, wherein the subject has a cancer.

94. The method of claim 89 or claim 90 or the kit of any of claims 91-93, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.


SEQUENCE LISTING (51 sequences)

SEQ ID NO: 1 - protein, 86 aa, synthetic construct
  UNSURE 1..86: note = IL2RG Common Gamma Chain Intracellular domain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, synthetic construct
  UNSURE 1..286: note = IL2RB intracellular domain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, synthetic construct
  UNSURE 1..195: note = IL7RB intracellular domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 285 aa, synthetic construct
  UNSURE 1..285: note = IL21RB intracellular domain
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 5 - protein, 107 aa, synthetic construct
  UNSURE 1..107: note = FKBP domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, synthetic construct [sequence omitted]

SEQ ID NO: 7 - protein, 93 aa, synthetic construct
  UNSURE 1..93: note = variant FRB domain (FRB mutant domain)
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 8 - protein, 21 aa, synthetic construct
  UNSURE 1..21: note = transmembrane (TM) domain
VVISVGSMGLIISLLCVYFWL

SEQ ID NO: 9 - protein, 19 aa, synthetic construct
  UNSURE 1..19: note = TM domain
VAVAGCVFLLISVLLLSGL

SEQ ID NO: 10 - protein, 25 aa, synthetic construct
  UNSURE 1..25: note = TM domain
PILLTISILSFFSVALLVILACVLW

SEQ ID NO: 11 - protein, 21 aa, synthetic construct
  UNSURE 1..21: note = TM domain
GWNPHLLLLLLLVIVFIPAFW

SEQ ID NO: 12 - protein, 21 aa, synthetic construct
  UNSURE 1..21: note = CD8a signal sequence
MALPVTALLLPLALLLHAARP

SEQ ID NO: 13 - DNA, 1554 bp, synthetic construct
  source 1..1554: mol_type = other DNA
  unsure 1..1554: note = nucleotide sequence encoding a CAR
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 517 aa, unidentified
  UNSURE 1..517: note = CAR amino acid sequence
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1560 bp, synthetic construct
  source 1..1560: mol_type = other DNA
  unsure 1..1560: note = nucleotide insert
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - protein, 496 aa, synthetic construct
  UNSURE 1..496: note = CAR amino acid sequence without the signal peptide
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 21 aa, synthetic construct
  UNSURE 1..21: note = CAR amino acid sequence signal peptide
MALPVTALLLPLALLLHAARP

SEQ ID NO: 18 - DNA, 20 bp, synthetic construct
  source 1..20: mol_type = other DNA
  unsure 1..20: note = B2M gRNA
ggccgagatgtctcgctccg

SEQ ID NO: 19 - DNA, 20 bp, synthetic construct
  source 1..20: mol_type = other DNA
  unsure 1..20: note = FKBP12 gRNA
cacgcaggtctggccgcgct

SEQ ID NO: 20 - DNA, 20 bp, synthetic construct
  source 1..20: mol_type = other DNA
  unsure 1..20: note = FKBP12 gRNA
tgctgagccgatgcgcgcgg

SEQ ID NO: 21 - DNA, 20 bp, synthetic construct
  source 1..20: mol_type = other DNA
  unsure 1..20: note = FKBP12 gRNA
ctcaccgtctcctggggaga

SEQ ID NO: 22 - DNA, 500 bp, synthetic construct
  source 1..500: mol_type = other DNA
  unsure 1..500: note = B2M 5 prime homology arm
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggc

SEQ ID NO: 23 - DNA, 500 bp, synthetic construct
  source 1..500: mol_type = other DNA
  unsure 1..500: note = B2M 3 prime homology arm
tggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 24 - DNA, 1189 bp, synthetic construct
  source 1..1189: mol_type = other DNA
  unsure 1..1189: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 25 - DNA, 1188 bp, synthetic construct
  source 1..1188: mol_type = other DNA
  unsure 1..1188: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 26 - DNA, 1205 bp, synthetic construct
  source 1..1205: mol_type = other DNA
  unsure 1..1205: note = EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 27 - DNA, 347 bp, synthetic construct
  source 1..347: mol_type = other DNA
  unsure 1..347: note = MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 28 - protein, 231 aa, synthetic construct
  UNSURE 1..231: note = RACR (g)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 29 - protein, 20 aa, synthetic construct
  UNSURE 1..20: note = Signal peptide
MPLGLLWLGLALLGALHAQA

SEQ ID NO: 30 - protein, 108 aa, synthetic construct
  UNSURE 1..108: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 31 - protein, 37 aa, synthetic construct
  UNSURE 1..37: note = IL2RG transmembrane domain and ectodomain portion
GSNTSKENPFLFALEAVVISVGSMGLIISLLCVYFWL

SEQ ID NO: 32 - DNA, 3354 bp, synthetic construct
  source 1..3354: mol_type = other DNA
  unsure 1..3354: note = RACR
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtg

SEQ ID NO: 33 - protein, 429 aa, synthetic construct
  UNSURE 1..429: note = RACR (b)
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 34 - protein, 21 aa, synthetic construct
  UNSURE 1..21: note = CD8a Signal peptide
MALPVTALLLPLALLLHAARP

SEQ ID NO: 35 - protein, 29 aa, synthetic construct
  UNSURE 1..29: note = IL2RB transmembrane domain and ectodomain portion
GKDTIPWLGHLLVGLSGAFGFIILVYLLI

SEQ ID NO: 36 - protein, 25 aa, synthetic construct
  UNSURE 1..25: note = IL2RB transmembrane domain
IPWLGHLLVGLSGAFGFIILVYLLI

SEQ ID NO: 37 - DNA, 753 bp, synthetic construct
  source 1..753: mol_type = other DNA
  unsure 1..753: note = RACR (g) Nucleotides
atgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagaca

SEQ ID NO: 38 - DNA, 1287 bp, synthetic construct
  source 1..1287: mol_type = other DNA
  unsure 1..1287: note = RACR (b) nucleotides
atggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtg

SEQ ID NO: 39 - DNA, 49 bp, synthetic construct
  source 1..49: mol_type = other DNA
  unsure 1..49: note = Synthetic polyA
aataaaagatctttattttcattagatctgtgtgttggttttttgtgtg

SEQ ID NO: 40 - DNA, 4354 bp, synthetic construct
  source 1..4354: mol_type = other DNA
  unsure 1..4354: note = RACR_B2M ssODN
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 41 - DNA, 66 bp, synthetic construct
  source 1..66: mol_type = other DNA
  unsure 1..66: note = PQR linker nucleotide
ggaagcggagcgacgaattttagtctactgaaacaagcgggagacgtggaggaaaaccctggacct

SEQ ID NO: 42 - protein, 22 aa, synthetic construct
  UNSURE 1..22: note = PQR linker
GSGATNFSLLKQAGDVEENPGP

SEQ ID NO: 43 - protein, 22 aa, synthetic construct
  UNSURE 1..22: note = F2A
VKQTLNFDLLKLAGDVESNPGP

SEQ ID NO: 44 - protein, 20 aa, synthetic construct
  UNSURE 1..20: note = E2A
QCTNYALLKLAGDVESNPGP

SEQ ID NO: 45 - protein, 24 aa, synthetic construct
  UNSURE 1..24: note = T2A
LEGGGEGRGSLLTCGDVEENPGPR

SEQ ID NO: 46 - protein, 18 aa, synthetic construct
  UNSURE 1..18: note = T2A
EGRGSLLTCGDVEENPGP

SEQ ID NO: 47 - protein, 22 aa, synthetic construct
  UNSURE 1..22: note = P2A
GSGATNFSLLKQAGDVEENPGP

SEQ ID NO: 48 - protein, 106 aa, synthetic construct
  UNSURE 1..106: note = FKBP domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 49 - protein, 108 aa, synthetic construct; identical to SEQ ID NO: 30
  UNSURE 1..108: note = FKBP domain

SEQ ID NO: 50 - protein, 90 aa, synthetic construct
  UNSURE 1..90: note = FRB domain
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 51 - protein, 230 aa, synthetic construct
  UNSURE 1..230: note = IL-9RA
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF